(19) United States
(12) Patent Application Publication
(10) Pub. No.: US 2006/0122197 A1
(43) Pub. Date: Jun. 8, 2006

(54) Amido compounds and their use as pharmaceuticals

(76) Inventors: Wenqing Yao, Kennett Square, PA (US); Jincong Zhuo, Boothwyn, PA (US); Brian W. Metcalf, Moraga, CA (US); Ding-Quan Qian, Newark, DE (US); Yanlong Li, Newark, DE (US)

Correspondence Address: COZEN O' CONNOR, P.C., 1900 MARKET STREET, PHILADELPHIA, PA, 19103-3508, US

(21) Appl. No.: 11/199,763

(22) Filed: Aug. 9, 2005

Related U.S. Application Data
(60) Provisional application No. 60/600,445, filed on Aug. 10, 2004.

(51) Int. Cl. A61K 31/506; A61K 31/4545; A61K 31/454; C07D 417/02; C07D 403/02; C07D 401/02

(52) U.S. Cl. 514/256; 514/318; 514/317; 514/326; 544/331; 546/194; 546/209; 546/224; 514/314; 514/319

(57) ABSTRACT

The present invention relates to inhibitors of 11-β hydroxyl steroid dehydrogenase type 1, antagonists of the mineralocorticoid receptor (MR), and pharmaceutical compositions thereof. The compounds of the invention can be useful in the treatment of various diseases associated with expression or activity of 11-β hydroxyl steroid dehydrogenase type 1 and/or diseases associated with aldosterone excess.

### CROSS REFERENCE TO RELATED APPLICATIONS

This application claims the benefit of U.S. Ser. No. 60/600,445, filed Aug. 10, 2004, the disclosure of which is incorporated herein by reference in its entirety.

### FIELD OF THE INVENTION

The present invention relates to modulators of 11-β hydroxyl steroid dehydrogenase type 1 (11βHSD1) and/or mineralocorticoid receptor (MR), compositions thereof and methods of using the same.

### BACKGROUND OF THE INVENTION

Glucocorticoids are steroid hormones that regulate fat metabolism, function and distribution. In vertebrates, glucocorticoids also have profound and diverse physiological effects on development, neurobiology, inflammation, blood pressure, metabolism and programmed cell death. In humans, the primary endogenously-produced glucocorticoid is cortisol. Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, driven by corticotropin releasing hormone (CRH) produced by the paraventricular nucleus of the hypothalamus. The HPA axis maintains circulating cortisol concentrations within restricted limits, with forward drive at the diurnal maximum or during periods of stress, and is rapidly attenuated by a negative feedback loop resulting from the ability of cortisol to suppress ACTH production in the anterior pituitary and CRH production in the hypothalamus.

Aldosterone is another hormone produced by the adrenal cortex; aldosterone regulates sodium and potassium homeostasis. Fifty years ago, a role for aldosterone excess in human disease was reported in a description of the syndrome of primary aldosteronism (Conn, (1955), J. Lab. Clin. Med. 45: 6-17). It is now clear that elevated levels of aldosterone are associated with deleterious effects on the heart and kidneys, and are a major contributing factor to morbidity and mortality in both heart failure and hypertension.

Two members of the nuclear hormone receptor superfamily, glucocorticoid receptor (GR) and mineralocorticoid receptor (MR), mediate cortisol function in vivo, while the primary intracellular receptor for aldosterone is the MR. These receptors are also referred to as ‘ligand-dependent transcription factors,’ because their functionality is dependent on the receptor being bound to its ligand (for example, cortisol); upon ligand-binding these receptors directly modulate transcription via DNA-binding zinc finger domains and transcriptional activation domains.

Historically, the major determinants of glucocorticoid action were attributed to three primary factors: 1) circulating levels of glucocorticoid (driven primarily by the HPA axis), 2) protein binding of glucocorticoids in circulation, and 3) intracellular receptor density inside target tissues. Recently, a fourth determinant of glucocorticoid function was identified: tissue-specific pre-receptor metabolism by glucocorticoid-activating and -inactivating enzymes. These 11-beta-hydroxysteroid dehydrogenase (11-β-HSD) enzymes act as pre-receptor control enzymes that modulate activation of the GR and MR by regulation of glucocorticoid hormones. To date, two distinct isozymes of 11-beta-HSD have been cloned and characterized: 11βHDS1 (also known as 11-beta-HSD type 1, 11betaHSD1, HSD11B1, HDL, and HSD11L) and 11βHSD2. 11βHSD1 and 11βHSD2 catalyze the interconversion of hormonally active cortisol (corticosterone in rodents) and inactive cortisone (11-dehydrocorticosterone in rodents). 11βHSD1 is widely distributed in rat and human tissues; expression of the enzyme and corresponding mRNA have been detected in lung, testis, and most abundantly in liver and adipose tissue. 11βHSD1 catalyzes both 11-beta-dehydrogenation and the reverse 11-oxoreduction reaction, although 11βHSD1 acts predominantly as a NADPH-dependent oxoreductase in intact cells and tissues, catalyzing the activation of cortisol from inert cortisone (Low et al. (1994) J. Mol. Endocrin. 13: 167-174) and has been reported to regulate glucocorticoid access to the GR. Conversely, 11βHSD2 expression is found mainly in mineralocorticoid target tissues such as kidney, placenta, colon and salivary gland, acts as an NAD-dependent dehydrogenase catalyzing the inactivation of cortisol to cortisone (Albiston et al. (1994) Mol. Cell. Endocrin. 105: R11-R17), and has been found to protect the MR from glucocorticoid excess, such as high levels of receptor-active cortisol (Blum, et al., (2003) Prog. Nucl. Acid Res. Mol. Biol. 75:173-216).

In vitro, the MR binds cortisol and aldosterone with equal affinity. The tissue specificity of aldosterone activity, however, is conferred by the expression of 11βHSD2 (Funder et al. (1988), Science 242: 583-585). The inactivation of cortisol to cortisone by 11βHSD2 at the site of the MR enables aldosterone to bind to this receptor in vivo. The binding of aldosterone to the MR results in dissociation of the ligand-activated MR from a multiprotein complex containing chaperone proteins, translocation of the MR into the nucleus, and its binding to hormone response elements in regulatory regions of target gene promoters. Within the distal nephron of the kidney, induction of serum and glucocorticoid inducible kinase-1 (sgk-1) expression leads to the absorption of Na⁺ ions and water through the epithelial sodium channel, as well as potassium excretion with subsequent volume expansion and hypertension (Bhargava et al., (2001), Endo 142: 1587-1594).

In humans, elevated aldosterone concentrations are associated with endothelial dysfunction, myocardial infarction, left ventricular atrophy, and death. In attempts to modulate these ill effects, multiple intervention strategies have been adopted to control aldosterone overactivity and attenuate the resultant hypertension and its associated cardiovascular consequences. Inhibition of angiotensin-converting enzyme (ACE) and blockade of the angiotensin type 1 receptor (AT1R) are two strategies that directly impact the rennin-angiotensin-aldosterone system (RAAS). However, although ACE inhibition and AT1R antagonism initially reduce aldosterone concentrations, circulating concentrations of this hormone return to baseline levels with chronic therapy (known as ‘aldosterone escape’). Importantly, co-administration of the MR antagonist Spironolactone or Eplerenone directly blocks the deleterious effects of this escape mechanism and dramatically reduces patient mortality (Pitt et al., New England J. Med. (1999), 341: 709-719; Pitt et al., New England J. Med. (2003), 348: 1309-1321). Therefore, MR antagonism may be an important treatment strategy for many patients with hypertension and cardiovascular disease, particularly those hypertensive patients at risk for target-organ damage.

Mutations in either of the genes encoding the 11-beta-HSD enzymes are associated with human pathology. For example, 11βHSD2 is expressed in aldosterone-sensitive tissues such as the distal nephron, salivary gland, and colonic mucosa where its cortisol dehydrogenase activity serves to protect the intrinsically non-selective MR from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralocorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1, a primary regulator of tissue-specific glucocorticoid bioavailability, and in the gene encoding a co-localized NADPH-generating enzyme, hexose 6-phosphate dehydrogenase (H6PD), can result in cortisone reductase deficiency (CRD), in which activation of cortisone to cortisol does not occur, resulting in adrenocorticotropin-mediated androgen excess. CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS) (Draper et al. (2003) Nat. Genet. 34: 434-439).

The importance of the HPA axis in controlling glucocorticoid excursions is evident from the fact that disruption of homeostasis in the HPA axis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Patients with Cushing's syndrome (a rare disease characterized by systemic glucocorticoid excess originating from the adrenal or pituitary tumors) or receiving glucocorticoid therapy develop reversible visceral fat obesity. Interestingly, the phenotype of Cushing's syndrome patients closely resembles that of Reaven's metabolic syndrome (also known as Syndrome X or insulin resistance syndrome) the symptoms of which include visceral obesity, glucose intolerance, insulin resistance, hypertension, type 2 diabetes and hyperlipidemia (Reaven (1993) Ann. Rev. Med. 44: 121-131). However, the role of glucocorticoids in prevalent forms of human obesity has remained obscure because circulating glucocorticoid concentrations are not elevated in the majority of metabolic syndrome patients. In fact, glucocorticoid action on target tissue depends not only on circulating levels but also on intracellular concentration, locally enhanced action of glucocorticoids in adipose tissue and skeletal muscle has been demonstrated in metabolic syndrome. Evidence has accumulated that enzyme activity of 11βHSD1, which regenerates active glucocorticoids from inactive forms and plays a central role in regulating intracellular glucocorticoid concentration, is commonly elevated in fat depots from obese individuals. This suggests a role for local glucocorticoid reactivation in obesity and metabolic syndrome.

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the latter, as well as b) the ability of 11βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local 11βHSD1-mediated conversion of inert glucocorticoid to active glucocorticoid can have profound influences whole body insulin sensitivity.

Based on this data, it would be predicted that the loss of 11βHSD1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Taken together, these transgenic mouse studies confirm a role for local reactivation of glucocorticoids in controlling hepatic and peripheral insulin sensitivity, and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Furthermore, it was recently reported that selective inhibitors of 11βHSD1 can ameliorate severe hyperglycemia in genetically diabetic obese mice. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, and/or hyperlipidemia. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). Thus, inhibition of 11βHSD1 is predicted to have multiple beneficial effects in the liver, adipose, and/or skeletal muscle, particularly related to alleviation of component(s) of the metabolic syndrome and/or obesity.

B. Pancreatic Function

Glucocorticoids are known to inhibit the glucose-stimulated secretion of insulin from pancreatic beta-cells (Billaudel and Sutter (1979) Horm. Metab. Res. 11: 555-560). In both Cushing's syndrome and diabetic Zucker fa/fa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release.

C. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11βHSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

D. Intra-ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

E. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawa et al. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11βHSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

F. Bone Disease

Glucocorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, and WO 2004/065351.

Antagonists of 11βHSD1 have been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS), therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1 and/or MR. The compounds, compositions and methods described herein help meet this and other needs.

### SUMMARY OF THE INVENTION

The present invention provides, inter alia, compounds of Formula Ia: 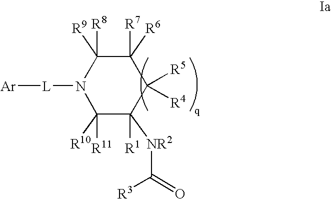
or pharmaceutically acceptable salts or prodrugs thereof, wherein constituent members are defined herein.

The present invention further provides compounds of Formula I: 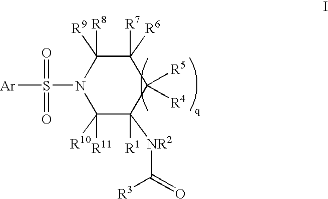
or pharmaceutically acceptable salts or prodrugs thereof, wherein constituent members are defined herein.

The present invention further provides compositions comprising compounds of the invention and a pharmaceutically acceptable carrier.

The present invention further provides methods of modulating 11βHSD1 or MR by contacting 11βHSD1 or MR with a compound of the invention.

The present invention further provides methods of inhibiting 11βHSD1 or MR by contacting 11βHSD1 or MR with a compound of the invention.

The present invention further provides methods of inhibiting the conversion of cortisone to cortisol in a cell by contacting the cell with a compound of the invention.

The present invention further provides methods of inhibiting the production of cortisol in a cell by contacting the cell with a compound of the invention.

The present invention further provides methods of treating diseases assocated with activity or expression of 11βHDS1 or MR.

### DETAILED DESCRIPTION

The present invention provides, inter alia, a compound of Formula Ia: 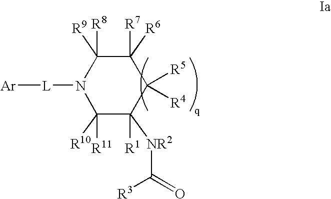
or pharmaceutically acceptable salt or prodrug thereof, wherein:

L is absent, S(O)₂, S(O), S, C(O), C(O)O, C(O)O-(C₁₋₃ alkylene), or C(O)NR^{L};

Ar is aryl or heteroaryl, each optionally substituted by 1, 2, 3, 4 or 5 -W-X-Y-Z;

R^{L} is H or C₁₋₆ alkyl;

R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

R² is H, C₁₋₆ alkyl, arylalkyl, heteroarylalkyl, cycloalkyl, cycloalkylalkyl, heterocycloalkyl or heterocycloalkylalkyl, each optionally substituted by 1, 2 or 3 R¹⁴;

R³ is H, C₁₋₆ alkyl, aryl, cycloalkyl, heteroaryl, heterocycloalkyl, each optionally substituted by 1, 2 or 3 -W′-X′-Y′-Z′;

or R³ is NR^{3a}R^{3b};

R^{3a} and R^{3b} are each, independently, H, C₁₋₆ alkyl, aryl, cycloalkyl, heteroaryl, heterocycloalkyl, each optionally substituted by 1, 2 or 3 -W′-X′-Y′-Z′;

or R^{3a} and R^{3b} together with the N atom to which they are attached form a 4-14 membered heterocycloalkyl group which is optionally substituted by 1, 2 or 3 -W′-X′-Y′-Z′;

R⁴, R⁵, R⁶, R⁷, R⁸, R⁹, R¹⁰ and R¹¹ are each, independently, H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

or R¹ and R² together with the carbon and nitrogen atoms to which they are attached form a 3-14 membered heterocycloalkyl group which is optionally substituted by 1, 2 or 3 R¹⁴;

or R¹ and R³ together with the carbon atoms to which they are attached and the intervening —NR²CO— moiety form a 4-14 membered heterocycloalkyl group which is optionally substituted by 1, 2 or 3 R¹⁴;

or R² and R³ together with the carbon and nitrogen atoms to which they are attached form a 3-14 membered heterocycloalkyl group which is optionally substituted by 1, 2 or 3 R¹⁴;

or R⁴ and R⁵ together with the carbon atom to which they are attached form a 3-14 membered cycloalkyl or heterocycloalkyl group which is optionally substituted by 1, 2 or 3 R¹⁴;

or R⁶ and R⁷ together with the carbon atom to which they are attached form a 3-14 membered cycloalkyl or heterocycloalkyl group which is optionally substituted by 1, 2 or 3 R¹⁴;

or R⁸ and R⁹ together with the carbon atom to which they are attached form a 3-14 membered cycloalkyl or heterocycloalkyl group which is optionally substituted by 1, 2 or 3 R¹⁴;

or R¹⁰ and R¹¹ together with the carbon atom to which they are attached form a 3-14 membered cycloalkyl or heterocycloalkyl group which is optionally substituted by 1, 2 or 3 R¹⁴;

or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;

or R⁶ and R⁸ together with the carbon atom to which they are attached form a 3-7 membered fused cycloalkyl group or 3-7 membered fused heterocycloalkyl group which is optionally substituted by 1, 2 or 3 R¹⁴;

R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{e}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

wherein two -W-X-Y-Z attached to the same atom optionally form a 3-14 membered cycloalkylk or 3-14 membered heterocycloalkyl group optionally substituted by 1, 2 or 3 -W″-X″-Y″-Z″;

wherein two -W′-X′-Y′-Z′ attached to the same atom optionally form a 3-14 membered cycloalkyl or 3-14 membered heterocycloalkyl group optionally substituted by 1, 2 or 3 -W″-X″-Y″-Z′;

wherein -W-X-Y-Z is other than H;

wherein -W′-X′-Y′-Z′ is other than H;

wherein -W″-X″-Y″-Z″ is other than H;

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl, wherein said C₁₋₆ alkyl, C₁₋₆ haloalkyl, C₂₋₆ alkenyl, C₂₋₆ alkynyl, aryl, cycloalkyl, heteroaryl or heterocycloalkyl; heterocycloalkyl, heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} and R^{f} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{e} and R^{f} together with the N atom to which they are attached form a 4-, 5-, 6- or 7-membered heterocycloalkyl group; and

q is 1 or 2.

In some embodiments, when L is absent and R² is methyl, then R³ is other than C₂₋₃ alkyl substituted by S(O)₂R^{b}.

In some embodiments, when L is absent and R³ is methyl, then R² is other than ethyl substituted by NR^{c′}R^{d′}.

In some embodiments, when L is S(O)₂ and Ar is 4-methylphenyl, then R³ is other than piperazin-1-yl which is 4-substituted by aryl.

In some embodiments, when L is S(O)₂ and q is 2, then Ar is other than aryl optionally substituted by 1, 2, 3, 4 or 5 -W-X-Y-Z.

In some embodiments, when L is C(O)NH and Ar is phenyl substituted by COOH, then R³ is other than heteroaryl substituted by 2 -W′-X′-Y′-Z′, or ethyl substituted by 2 -W′-X′-Y′-Z′.

In some embodiments, when L is C(O), C(O)O, or C(O)O-(C₁₋₃ alkylene) then R³ is other than substituted or unsubstituted piperidin-3-yl.

In some embodiments, when L is C(O), C(O)O, or C(O)O-(C₁₋₃ alkylene) then R³ is other than substituted or unsubstituted piperidinyl.

In some embodiments, R³ is other than piperidin-3-yl which is N-substituted by one —C(O)-(C₁₋₄ alkyl) or one —C(O)O(C₁₋₄ alkyl).

In some embodiments, R³ is other than N-substituted piperidin-3-yl.

In some embodiments, R³ is other than N-substituted pyrrolidin-3-yl.

In some embodiments, R³ is other than substituted piperidin-3-yl.

In some embodiments, R³ is other than substituted pyrrolidin-3-yl.

In some embodiments, R³ is other than substituted piperidinyl.

In some embodiments, R³ is other than substituted pyrrolidinyl.

In some embodiments, R³ is other than substituted 6-membered heterocycloalkyl.

In some embodiments, L is absent, S(O)₂, C(O)NR^{L}, or C(O)O-(C₁₋₃ alkylene).

In some embodiments, L is absent, S(O)₂, or C(O)NR^{L}.

In some embodiments, L is absent or S(O)₂.

In some embodiments, L is S(O)₂.

In some embodiments, L is absent.

In some embodiments, L is C(O).

In some embodiments, L is C(O)NR^{L}.

In some embodiments, L is C(O)NH.

In some embodiments, L is C(O)O-(C₁₋₃ alkylene).

In some embodiments, L is C(O)O—CH₂.

In some embodiments, the compound has Formula IIa:

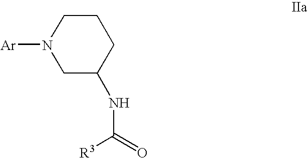

In some embodiments, the compound has Formula IIa and Ar is phenyl, pyridyl, pyrimidinyl, thiazolyl, each optionally substituted with 1 or 2 -W-X-Y-Z.

In some embodiments, the compound has Formula IIa Ar is phenyl, pyridyl, pyrimidinyl, thiazolyl, each optionally substituted with 1 or 2 halo, nitro, cyano, amino, C₁₋₄ alkyl, C₁₋₄ alkoxy, C₁₋₄ haloalkyl, C₁₋₄ haloalkoxy, dialkylaminocarbonyl, dialkylaminocarbonylalkyloxy, cycloalkylcarbonylamino, cycloalkylcarbonyl(alkyl)amino, alkoxycarbonylamino, alkoxycarbonyl, alkylsulfonyl, alkylsulfonylamino, cycloalkylalkylcarbonylamino, aryl, heteroaryl, heterocycloalkyl, arylalkyloxy, cycloalkyloxy, heterocycloalkyloxy, acylamino, acyl(alkyl)amino, 1,2,3,6-tetrahydropyridinyl substituted by alkoxycarbonyl, 2-oxopiperidinyl, or 2-oxopyrrolidinyl;

wherein said aryl, heteroaryl, heterocycloalkyl, and heterocycloalkyloxy, are each optionally substituted by one or more halo, cyano, C₁₋₄ alkoxy, acyl, acylamino, alkylsulfonyl, cycloalkylaminocarbonyl, alkoxycarbonyl, or aminocarbonyl.

In some embodiments, the compound has Formula IIa and R³ is cyclopropyl, cyclobutyl, cyclopentyl, cyclohexyl, cycloheptyl, adamantyl, bicyclo[3.2.1]octanyl, norbornyl, 1,2,3,4-tetrahydronaphthyl, azepan-7-on-yl, 8-aza-bicyclo[3.2.1]octanyl, indolyl, quinolinyl, indol-3-ylmethyl, or phenyl, each optionally substituted by 1 or 2 -W′-X′-Y′-Z′.

In some embodiments, the compound has Formula IIa and R³ is cyclopropyl, cyclobutyl, cyclopentyl, cyclohexyl, cycloheptyl, adamantyl, bicyclo[3.2.1]octanyl, norbornyl, 1,2,3,4-tetrahydronaphthyl, azepan-7-on-yl, 8-aza-bicyclo[3.2.1]octanyl, or phenyl, each optionally substituted by 1 or 2 halo, OH, C₁₋₄ alkyl, C₁₋₄ alkoxy, hydroxylalkyl, aryl, aryloxy, heteroaryl, heteroarylalkyl, or alkylcarbonyloxy;

wherein said aryl, heteroaryl, heteroarylalkyl is optionally substituted by 1 or 2 C₁₋₄ alkyl or heterocycloalkyl optionally substituted by alkoxycarbonyl.

In some embodiments, the compound has Formula IIIa:

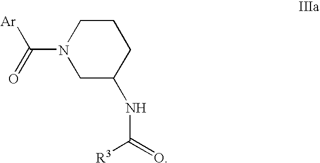

In some embodiments, the compound has Formula IVa:

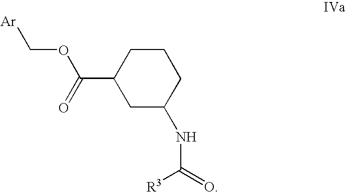

In some embodiments, the compound has Formula Va:

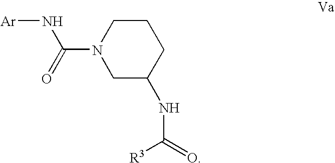

In some embodiments, when the compound has Formula Va. In some embodiments of compounds of Formulat IV, when Ar is phenyl substituted by COOH, then R³ is other than heteroaryl substituted by 2 -W′-X′-Y′-Z′, or ethyl substituted by 2 -W′-X′-Y′-Z′.

In some embodiments, the compound has Formula I: 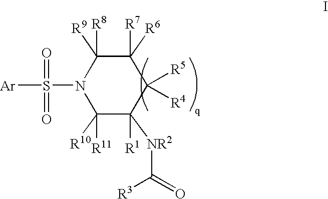
or pharmaceutically acceptable salt or prodrug thereof, wherein:

Ar is aryl or heteroaryl, each optionally substituted by 1, 2, 3, 4 or 5 -W-X-Y-Z;

R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀alkyl, C₁₋₁₀haloalkyl, C₂₋₁₀alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

R² is H, C₁₋₆ alkyl, arylalkyl, heteroarylalkyl, cycloalkyl, cycloalkylalkyl, heterocycloalkyl or heterocycloalkylalkyl, each optionally substituted by 1, 2 or 3 R¹⁴;

R³ is H, C₁₋₆ alkyl, aryl, cycloalkyl or heteroaryl, each optionally substituted by 1, 2 or 3 -W′-X′-Y′-Z′;

R⁴, R⁵, R⁶, R⁷, R⁸, R⁹, R¹⁰ and R¹¹ are each, independently, H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

or R¹ and R² together with the carbon and nitrogen atoms to which they are attached form a 3-14 membered heterocycloalkyl group which is optionally substituted by 1, 2 or 3 R¹⁴;

or R¹ and R³ together with the carbon atoms to which they are attached and the intervening —NR²CO— moiety form a 4-14 membered heterocycloalkyl group which is optionally substituted by 1, 2 or 3 R¹⁴;

or R² and R³ together with the carbon and nitrogen atoms to which they are attached form a 3-14 membered heterocycloalkyl group which is optionally substituted by 1, 2 or 3 R¹⁴;

or R⁴ and R⁵ together with the carbon atom to which they are attached form a 3-14 membered cycloalkyl or 3-14 membered heterocycloalkyl group which is optionally substituted by 1, 2 or 3 R¹⁴;

or R⁶ and R⁷ together with the carbon atom to which they are attached form a 3-14 membered cycloalkyl or 3-14 membered heterocycloalkyl group which is optionally substituted by 1, 2 or 3 R¹⁴;

or R⁸ and R⁹ together with the carbon atom to which they are attached form a 3-14 membered cycloalkyl or 3-14 membered heterocycloalkyl group which is optionally substituted by 1, 2 or 3 R¹⁴;

or R¹⁰ and R¹¹ together with the carbon atom to which they are attached form a 3-14 membered cycloalkyl or 3-14 membered heterocycloalkyl group which is optionally substituted by 1, 2 or 3 R¹⁴;

or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;

or R⁶ and R⁸ together with the carbon atom to which they are attached form a 3-7 membered fused cycloalkyl group or 3-7 membered fused heterocycloalkyl group which is optionally substituted by 1, 2 or 3 R¹⁴;

R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

Z, Z′ and Z″ are each, independently, H, halo, CN, NO₂, OH, C₁₋₄ alkoxy, C₁₋₄ haloalkoxy, amino, C₁₋₄ alkylamino or C₂₋₈dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

wherein two -W-X-Y-Z attached to the same atom optionally form a 3-14 membered cycloalkyl or heterocycloalkyl group optionally substituted by 1, 2 or 3 -W″-X″-Y″-Z″;

wherein two -W′-X′-Y′-Z′ attached to the same atom optionally form a 3-14 membered cycloalkyl or heterocycloalkyl group optionally substituted by 1, 2 or 3 -W″-X″-Y″-Z″;

wherein -W-X-Y-Z is other than H;

wherein -W′-X′-Y′-Z′ is other than H;

wherein -W″-X″-Y″-Z″ is other than H;

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl, wherein said C₁₋₆ alkyl, C₁₋₆ haloalkyl, C₂₋₆ alkenyl, C₂₋₆ alkynyl, aryl, cycloalkyl, heteroaryl or heterocycloalkyl; heterocycloalkyl, heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} and R^{f} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{e} and R^{f} together with the N atom to which they are attached form a 4-, 5-, 6- or 7-membered heterocycloalkyl group; and

q is 1 or 2.

In some embodiments, Ar is aryl optionally substituted by 1, 2, 3, 4 or 5 -W-X-Y-Z.

In some embodiments, Ar is aryl optionally substituted by 1, 2 or 3 -Z.

In some embodiments, Ar is phenyl or naphthyl, each optionally substituted by 1, 2, 3, 4 or 5 -W-X-Y-Z.

In some embodiments, Ar is phenyl or naphthyl, each optionally substituted by 1, 2 or 3 -Z.

In some embodiments, Ar is phenyl or naphthyl, each optionally substituted by 1, 2 or 3 halo; nitro; cyano; C₁₋₄ alkyl; C₁₋₄ haloalkyl; C₁₋₄ alkoxy; C₁₋₄ haloalkoxy; dialkylamino; dialkylaminocarbonyl; alkylsulfonyl; cycloalkyloxy; heteroaryloxy; aryloxy; cycloalkyl; heterocycloalkyl; phenyl optionally substituted by one or more halo, cyano, C₁₋₄ alkyl, C₁₋₄ alkoxy, or —NHC(O)-(C₁₋₄ alkyl); or pyridyl optionally substituted by one or more halo, cyano, C₁₋₄ alkyl, C₁₋₄ alkoxy, or —NHC(O)-(C₁₋₄ alkyl).

In some embodiments, Ar is phenyl or naphthyl, each optionally substituted by 1, 2 or 3 halo, nitro, cyano, C₁₋₄ alkyl, C₁₋₄ alkoxy, C₁₋₄ haloalkyl, C₁₋₄ haloalkoxy, —O-aryl, —O-heteroaryl, NHC(O)-(C₁₋₄ alkyl), or SO₂-(C₁₋₄ alkyl).

In some embodiments, Ar is phenyl or naphthyl, each optionally substituted by 1, 2 or 3 C₁₋₄ alkyl or aryloxy.

In some embodiments, Ar is heteroaryl optionally substituted by 1, 2, 3, 4 or 5 -W-X-Y-Z.

In some embodiments, Ar is heteroaryl optionally substituted by 1, 2 or 3 -Z.

In some embodiments, Ar is pyridyl, pyrimidinyl, thienyl, thiazolyl, quinolinyl, 2,1,3-benzoxadiazolyl, isoquinolinyl or isoxazolyl, each optionally substituted by 1, 2, 3, 4 or 5 -W-X-Y-Z.

In some embodiments, Ar is pyridyl, thienyl, or isoxazolyl, each optionally substituted by 1, 2, 3, 4 or 5 -W-X-Y-Z.

In some embodiments, Ar is pyridyl, quinolinyl, 2,1,3-benzoxadiazolyl, isoquinolinyl, thienyl or isoxazolyl, each optionally substituted by 1, 2 or 3 -Z.

In some embodiments, Ar is pyridyl, thienyl or isoxazolyl, each optionally substituted by 1, 2 or 3 -Z.

In some embodiments, Ar is pyridyl, quinolinyl, 2,1,3-benzoxadiazolyl, isoquinolinyl, thienyl or isoxazolyl, each optionally substituted by 1, 2 or 3 halo, C₁₋₄ alkyl or aryloxy.

In some embodiments, q is 1.

In some embodiments, -W-X-Y-Z is halo, nitro, cyano, OH, C₁₋₄ alkyl, C₁₋₄ haloalkyl, C₁₋₄ haloalkoxy, amino, C₁₋₄ alkoxy, cycloalkylcarbonylamino, alkoxycarbonylamino, alkylsulfonylamino, cycloalkylalkylcarbonylamino, acyl(alkyl)amino, alkylamino, dialkylamino, dialkylaminosulfonyl, dialkylaminocarbonyl, dialkylaminocarbonylalkyloxy, alkylcarbonyl(alkyl)amino, cycloalkylcarbonyl(alkyl)amino, alkoxycarbonyl(alkyl)amino, alkoxycarbonyl, alkylsulfonyl, arylsulfonyl, aryl, cycloalkyl, heteroaryl, heterocycloalkyl, aryloxy, cycloalkyloxy, heteroaryloxy, heterocycloalkyloxy, arylalkyloxy, acylamino, 1,2,3,6-tetrahydropyridinyl substituted by alkoxycarbonyl, 2-oxopiperidinyl, or 2-oxopyrrolidinyl;

wherein said aryl, cycloalkyl, heteroaryl, heterocycloalkyl, arylalkyloxy, or heterocycloalkyloxy are optionally substituted by 1 or more halo, C₁₋₄ alkyl, OH, C₁₋₄ alkoxy, cycloalkylaminocarbonyl, alkoxycarbonyl, cyano, acyl, acylamino, alkylsulfonyl, amino, alkylamino, dialkylamino, or aminocarbonyl.

In some embodiments, -W′-X′-Y′-Z′ is halo, OH, cyano, nitro, C₁₋₄ alkyl, C₁₋₄ alkoxy, C₁₋₄ haloalkyl, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, or arylsulfonyl;

wherein said aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, is optionally substituted by 1 or 2 halo, OH, cyano, nitro, C₁₋₄ alkyl, C₁₋₄ alkoxy, C₁₋₄ haloalkyl, C₁₋₄ haloalkoxy, amino, alkylamino, dialkylamino, hydroxyalkyl, or alkoxycarbonyl.

In some embodiments, -W″-X″-Y″-Z″ is halo, OH, cyano, nitro, C₁₋₄ alkyl, C₁₋₄ alkoxy, C₁₋₄ haloalkyl, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, or arylsulfonyl;

In some embodiments, q is 1.

In some embodiments, R³ is C₁₋₆ alkyl optionally substituted by 1, 2 or 3 -W′-X′-Y′-Z′.

In some embodiments, R³ is C₁₋₆ alkyl optionally substituted by 1 or 2 aryl.

In some embodiments, R³ is C₁₋₆ alkyl.

In some embodiments, R³ is aryl, cycloalkyl, heteroaryl or heterocycloalkyl, each optionally substituted by 1, 2 or 3 -W′-X′-Y′-Z′.

In some embodiments, R³ is aryl, cycloalkyl, or heteroaryl, each optionally substituted by 1, 2 or 3 -W′-X′-Y′-Z′.

In some embodiments, R³ is C₁₋₄ alkyl, aryl, cycloalkyl, heteroaryl or heterocycloalkyl, each optionally substituted by 1, 2 or 3 halo, C₁₋₄ alkyl, C₁₋₄ haloalkyl, C₁₋₄ alkoxy, phenyl, phenyl substituted by halo, phenyloxy, pyridyl, acyl, alkoxycarbonyl, alkylsulfonyl, arylsulfonyl, or arylsulfonyl optionally substituted by 1 or 2 halo or C₁₋₄ alkyl.

In some embodiments, R³ is aryl, cycloalkyl, or heteroaryl, each optionally substituted by 1, 2 or 3 halo, C₁₋₄ alkyl, C₁₋₄ haloalkyl, C₁₋₄ alkoxy, C₁₋₄ haloalkoxy, C₂₋₈ alkoxyalkyl, phenyl, phenyloxy, pyridyl, or azepan-2-on-yl.

In some embodiments, R³ is aryl or cycloalkyl, each optionally substituted by 1, 2 or 3 -W′-X′-Y′-Z′.

In some embodiments, R³ is cycloheptyl, cyclohexyl, cyclopentyl, cyclopropyl, 1,2,3,4-tetrahydronaphthalenyl, norbornyl, or adamantyl, each optionally substituted by 1, 2 or 3 -W′-X′-Y′-Z′.

In some embodiments, R³ is cycloheptyl, cyclohexyl, cyclopentyl, cyclopropyl or adamantyl, each optionally substituted by 1, 2 or 3 -W′-X′-Y′-Z′.

In some embodiments, R³ is cycloheptyl, cyclohexyl, cyclopentyl, cyclopropyl or adamantyl, each optionally substituted by 1, 2 or 3 -Z′.

In some embodiments, R³ is cycloheptyl, cyclohexyl, cyclopentyl, cyclopropyl or adamantyl, each optionally substituted by 1, 2 or 3 CN, OH, C₁₋₄ alkoxy, C₁₋₆ alkyl, aryl, or aryl substituted by halo.

In some embodiments, R³ is cycloheptyl, cyclohexyl, cyclopentyl, cyclopropyl or adamantyl, each optionally substituted by 1, 2 or 3 OH, C₁₋₄ alkoxy, C₁₋₆ alkyl, aryl, or aryl substituted by halo.

In some embodiments, R³ is adamantyl optionally substituted by OH.

In some embodiments, R³ is phenyl or naphthyl, each optionally substituted by 1, 2 or 3 -W′-X′-Y′-Z′.

In some embodiments, R³ is phenyl or naphthyl, each optionally substituted by 1, 2 or 3 -Z′.

In some embodiments, R³ is phenyl or naphthyl, each optionally substituted by 1, 2 or 3 halo, C₁₋₄ alkyl, C₁₋₄ haloalkyl, C₁₋₄ alkoxy, C₁₋₄ haloalkoxy, C₂₋₈ alkoxyalkyl, aryl, aryloxy, pyridyl, or azepan-2-on-yl.

In some embodiments, R³ is phenyl or naphthyl, each optionally substituted by 1, 2 or 3 halo, C₁₋₄ alkyl, C₁₋₄ alkoxy, C₁₋₄ haloalkyl, aryl or aryloxy.

In some embodiments, R³ is heteroaryl or heterocycloalkyl, each optionally substituted by 1, 2 or 3 -W′-X′-Y′-Z′.

In some embodiments, R³ is piperidinyl optionally substituted by 1, 2 or 3 -W′-X′-Y′-Z′.

In some embodiments, R³ is piperidinyl optionally substituted by 1, 2 or 3 -Z′.

In some embodiments, R³ is piperidinyl optionally substituted by 1, 2 or 3 CO-(C₁₋₄ alkyl), C(O)O-(C₁₋₄ alkyl), SO₂-(C₁₋₄ alkyl), SO₂-aryl or SO₂-(aryl substituted by 1 or 2 halo or C₁₋₄ alkyl).

In some embodiments, R³ is piperidinyl optionally substituted by 1, 2 or 3 SO₂-(C₁₋₄ alkyl), SO₂-aryl or SO₂-(aryl substituted by 1 or 2 halo or C₁₋₄ alkyl).

In some embodiments, R³ is pyridyl optionally substituted by 1, 2 or 3 -W′-X′-Y′-Z′.

In some embodiments, R³ is pyridyl optionally substituted by 1, 2 or 3 -Z′.

In some embodiments, R³ is pyridyl.

In some embodiments, R³ is 8-aza-bicyclo[3.2.1]octanyl, indolyl, morpholino, S-oxo-thiomorpholino, S,S-dioxo-thiomorpholino, or thiomorpholino, each optionally substituted by 1, 2 or 3 -W′-X′-Y′-Z′.

In some embodiments, R³ is 8-aza-bicyclo[3.2.1]octanyl, indolyl, morpholino, S-oxo-thiomorpholino, S,S-dioxo-thiomorpholino, or thiomorpholino, each optionally substituted by 1, 2 or 3 -Z′.

In some embodiments, R⁴, R⁵, R⁶, R⁷, R⁸, R⁹, R¹⁰ and R¹¹ are each H.

In some embodiments, R¹ is H.

In some embodiments, R² is H.

In some embodiments the conpound has Formula II:

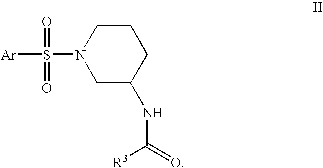

In some embodiments the compound has Formula II and Ar is phenyl, naphthyl, pyridyl, thienyl, isoxazolyl, quinolinyl, isoquinolinyl, or 2,1,3-benzoxadiazolyl, each optionally substituted with 1 or 2 halo, cyano, nitro, C₁₋₄ alkyl, C₁₋₄ haloalkyl, C₁₋₄ alkoxy, C₁₋₄ haloalkoxy, aryloxy, heteroaryloxy, acylamino, alkylsulfonyl, or dialkylamino.

In some embodiments the compound has Formula II and R³ is C₁₋₄ alkyl, cyclopropyl, cyclobutyl, cyclopentyl, cyclohexyl, cycloheptyl, adamantyl, phenyl, naphthyl, pyridyl, piperidinyl, morpholino, S-oxo-thiomorpholino, S,S-dioxo-thiomorpholino, thiomorpholino, or 8-aza-bicyclo[3.2.1]octanyl, each optionally substituted by 1 or 2 OH; C₁₋₄ alkyl; C₁₋₄ alkoxy; C₁₋₄ haloalkyl; phenyl; phenyloxy; arylsulfonyl optionally subsustituted by 1 or 2 halo or C₁₋₄ alkyl; chlorophenyl; alkylcarbonyl; alkoxycarbonyl; or alkylsulfonyl.

In some embodiments the compound has Formula I;

Ar is aryl or heteroaryl, each optionally substituted by 1, 2, 3, 4 or 5 -W-X-Y-Z;

R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₁₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

R² is H or C₁₋₆ alkyl;

R³ is H, C₁₋₆ alkyl, aryl, cycloalkyl, heteroaryl or heterocycloalkyl, each optionally substituted by 1, 2 or 3 -W′-X′-Y′-Z′;

R⁴, R⁵, R⁶, R⁷, R⁸, R⁹, R¹⁰ and R¹¹ are each, independently, H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3R¹⁴.

In some embodiments the compound has Formula I;

Ar is aryl or heteroaryl, each optionally substituted by 1, 2, 3, 4 or 5 -W-X-Y-Z;

R¹ is H;

R² is H;

R³ is C₁₋₆ alkyl, aryl, cycloalkyl, heteroaryl or heterocycloalkyl, each optionally substituted by 1, 2 or 3 -W′-X′-Y′-Z′; and

R⁴, R⁵, R⁶, R⁷, R⁸, R⁹, R¹⁰ and R¹¹ are each H.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylene” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles including cyclized alkyl, alkenyl, and alkynyl groups where one or more of the ring-forming carbon atoms is replaced by a heteroatom such as an O, N, or S atom. Hetercycloalkyl groups can be mono or polycyclic (e.g., both fused and spiro systems). Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used here, “haloalkoxy” refers to an —O-haloalkyl group. An example haloalkoxy group is OCF₃.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to an alkyl group substituted by a heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “dialkylaminocarbonyl” refers to a carbonyl group substituted by a dialkylamino group.

As used herein, “dialkylaminocarbonylalkyloxy” refers to an alkyloxy (alkoxy) group substituted by a carbonyl group which in turn is substituted by a dialkylamino group.

As used herein, “cycloalkylcarbonyl(alkyl)amino” refers to an alkylamino group substituted by a carbonyl group (on the N atom of the alkylamino group) which in turn is substituted by a cycloalkyl group. The term “cycloalkylcarbonylamino” refers to an amino group substituted by a carbonyl group (on the N atom of the amino group) which in turn is substituted by a cycloalkyl group. The term “cycloalkylalkylcarbonylamino” refers to an amino group substituted by a carbonyl group (on the N atom of the amino group) which in turn is substituted by a cycloalkylalkyl group.

As used herein, “alkoxycarbonyl(alkyl)amino” refers to an alkylamino group substituted by an alkoxycarbonyl group on the N atom of the alkylamino group. The term “alkoxycarbonylamino” refers to an amino group substituted by an alkoxycarbonyl group on the N atom of the amino group.

As used herein “alkoxycarbonyl” refers to a carbonyl group substituted by an alkoxy group.

As used herein, “alkylsulfonyl” refers to a sulfonyl group substituted by an alkyl group. The term “alkylsulfonylamino” refers to an amino group substituted by an alkylsulfonyl group.

As used herein, “arylsulfonyl” refers to a sulfonyl group substituted by an aryl group.

As used herein, “dialkylaminosulfonyl” refers to a sulfonyl group substituted by dialkylamino.

As used herein, “arylalkyloxy” refers to —O-arylalkly. An example of an arylalkyloxy group is benzyloxy.

As used heren, “cycloalkyloxy” refers to —O-cycloalkyl. An example of a cycloalkyloxy group is cyclopenyloxyl.

As used herein, “heterocycloalkyloxy” refers to —O-heterocycloalkyl.

As used herein, “heteroaryloxy” refers to —O-heteroaryl. An example is pyridyloxy.

As used herein, “acylamino” refers to an amino group substituted by an alkylcarbonyl (acyl) group. The term “acyl(alkyl)amino” refers to an amino group substituted by an alkylcarbonyl (acyl) group and an alkyl group.

As used herein, “alkylcarbonyl” refers to a carbonyl group substituted by an alkyl group.

As used herein, “cycloalkylaminocarbonyl” refers to a carbonyl group substituted by an amino group which in turn is substituted by a cycloalkyl group.

As used herein, “aminocarbonyl” refers to a carbonyl group substituted by an amino group (i.e., CONH₂).

As used herein, “hydroxyalkyl” refers to an alkyl group substituted by a hydroxyl group. An example is —CH₂OH.

As used herein, “alkylcarbonyloxy” refers to an oxy group substituted by a carbonyl group which in turn is substituted by an alkyl group.

As used herein, “N-substituted piperidin-3-yl” refers to a moiety having the formula: 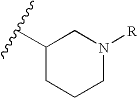
wherein R is any moiety other than H.

As used herein, “4-substituted piperazin-1-yl” refers to a moiety having the formula: 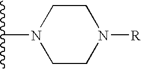
wherein R is any moiety other than H. In general, the terms “substitute” or “substitution” refer to replacing a hydrogen with a non-hydrogen moiety.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms, such as keto-enol tautomers.

Compounds of the invention can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

A series of N-(piperidin-3-yl)carboxamides of formula 4 can be prepared by the method outlined in Scheme 1. 1-(tert-Butoxycarbonyl)-3-amino-piperidine 1 can be coupled to acid chloride R³COCl in the presence of a base such as Hunig's base or potassium carbonate to provide the desired product 2. The Boc protecting group of 2 can be removed by treatment with HCl in 1,4-dioxane to afford the amino salt 3, which can be directly coupled with the appropriate chloride ArLCl to give the final compounds with formula 4. Alternatively, ureas having the general structure of 4′ can be prepared via the activated p-nitro-carbamate 3′ or by reaction of piperidine 3 with the appropriate isocyanate.

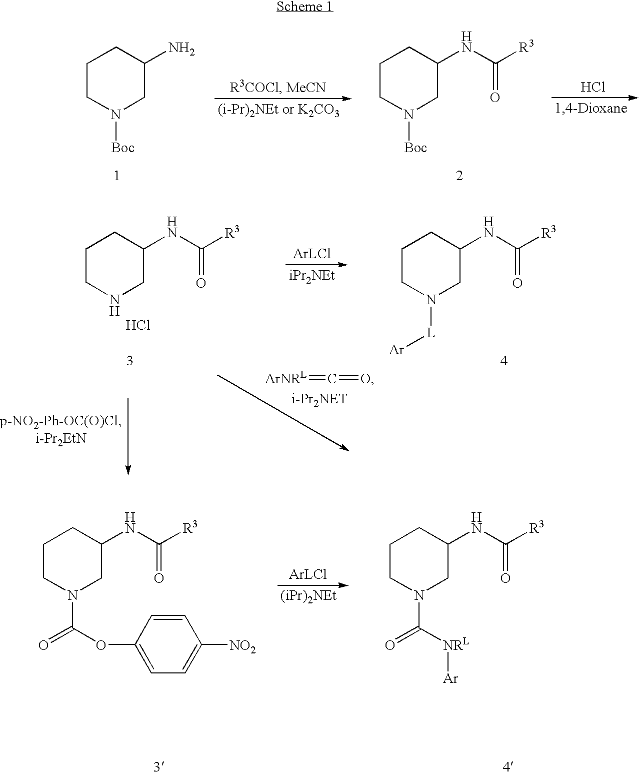

Alternatively, the same series of N-(piperidin-3-yl)carboxamides of formula 4 can be prepared in a similar fashion as described above but with a change the coupling sequences as shown in Scheme 2.

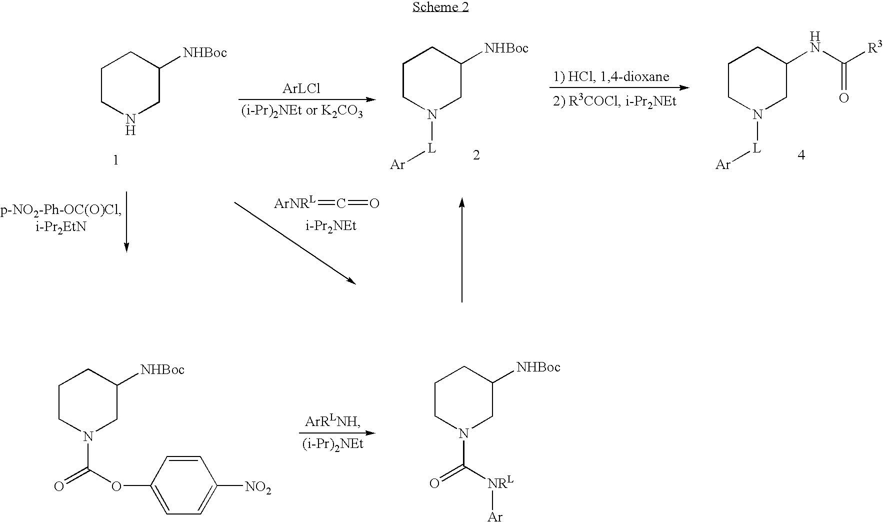

Alternatively, the same series of N-(piperidin-3-yl)carboxamides of formula 4 can be prepared by the method outlined in Scheme 3. The 3-amino-piperidine derivative 5 can be coupled to a carboxylic acid using a coupling reagent such as BOP in the presence of a suitable base such as N-methylmorpholine and in a suitable solvent such as DMF to provide the desired final product 4 according to Scheme 3.

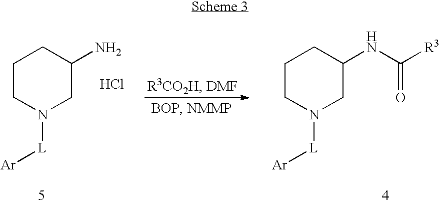

A series of N-(piperidine-3-yl)carboxamides of formula 6 can be prepared by the method outlined in Scheme 4. Compound 5 can be coupled to N-Boc-piperidinyl carboxylic acid 7 using a coupling reagent such as BOP in the presence of a suitable base such as N-methylmorpholine to afford an amido compound of formula 8. The Boc group of compound 8 can be removed by treatment with HCl in 1,4-dioxane to afford an amine compound of formula 9. The amine compound of formula 9 can be coupled with a compound RX to afford the desired product of formula 6, wherein X is a leaving group such as halide and RX can be sulfonyl chlorides, acid chlorides, alkyl chloroformates, or alkyl bromides.

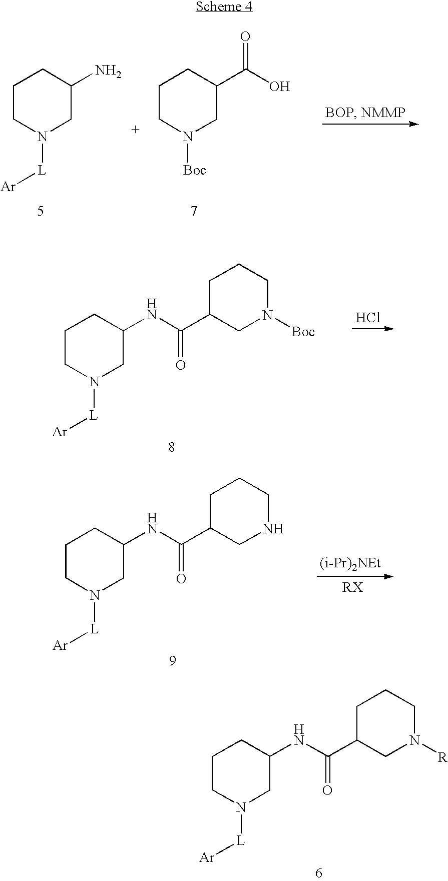

A series of 5-substituted 3-aminopiperidines of formula 10 can be prepared according to a method outlined in Scheme 5. Boc-protecting of L-Glutamic acid dimethyl ester 11 with di-tert-butyl dicarbonate gives N-Boc compound 12. Treatment of compound 12 with a compound RX such as alkyl bromide or alkyl iodide in the presence of suitable base such as sodium hydride, LDA or LiHMDS and in a suitable solvent such as DMF or THF, provides 4-alkyl dimethyl ester 13. Reduction of the ester group with suitable reducing reagents such as NaBH₄/CaCl₂ affords a di-OH compound 14. The hydroxyl groups of compound 14 can be converted to a better leaving group such as OMs by reacting with MsCl under basic conditions to afford a compound of 15. The desired 5-substituted 3-aminopiperidines 10 can be prepared by treatment of compound 15 with benzylamine followed by palladium catalytic hydrogenation.

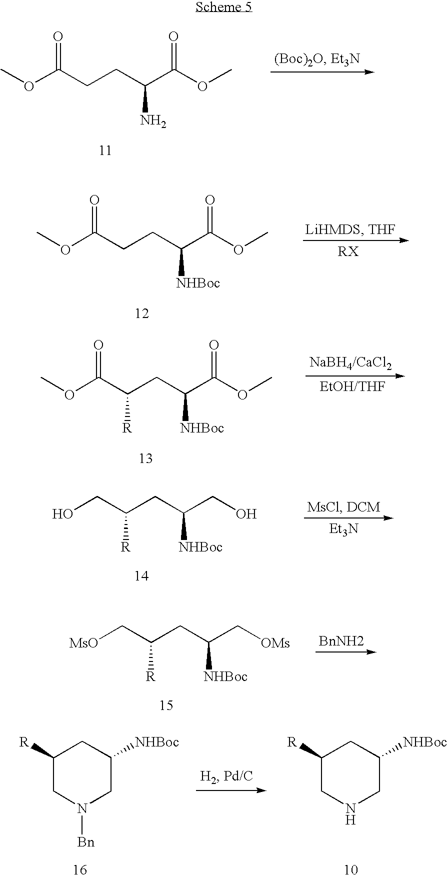

A series of spiro-3-aminopiperidines of formula 17 can be prepared in similar manners as shown in Scheme 6 wherein r can be 1-5. A diester compound 12 can react with a dihalide compound such as a dibromoalkyl compound in a suitable solvent such as THF, and in the presence of a suitable base such as LiHMDS to afford a cycloalkyl compound 18. The ester groups of compound 18 can be reduced by suitable reducing reagents such as a combination of NaBH₄/CaCl₂ in a suitable solvent such as EtOH/THF to afford a di-OH compound of 19. A spiro compound 17 can be obtained from the compound 19 by using similar procedures to those outlined in Scheme 5.

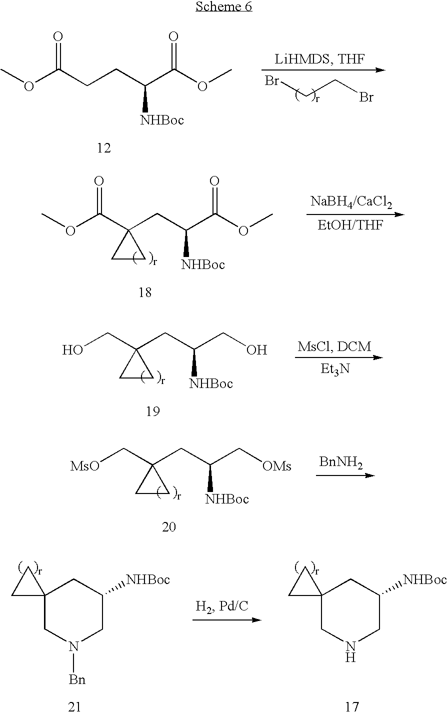

A series of 3-substituted-3-aminopiperidines of formula 22 can be prepared according to the method outlined in Scheme 7 wherein R can be alkyl, aryl, arylalkyl, cycloalkyl or cycloalkylalky. A ketone compound 23 can be treated with TsNH₂ to give an imino compound 24. The compound 24 is then reacted with a Grignard reagent such as RMgBr to afford a Ts-protected-amine compound 25. The Ts group of compound 25 can be removed by PhSH to afford compound 26. The amino group is then protected by Boc group using (Boc)₂O in the presence a suitable base such as triethylamine to give a Boc-protected compound 27. The Bn group of compound 27 is removed by hydrogenation with palladium as catalyst to afford the desired peridine compound 22.

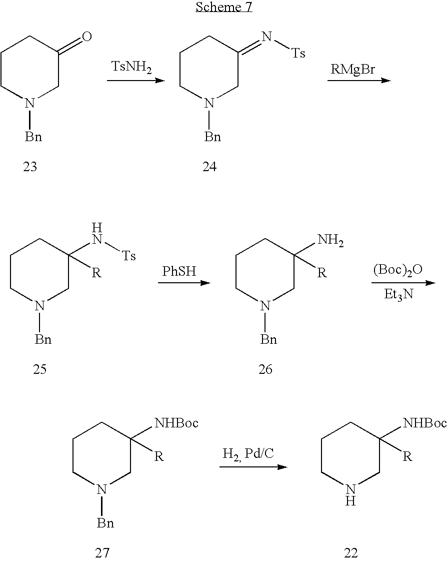

Tertiary amides of formula 28 can be prepared as shown in Scheme 8. The reductive amination of the 3-aminopiperidines 5 with a suitable aldehyde (R′ is, e.g., alkyl, aryl, heteroaryl, cycloalkyl, heterocycloalkyl, arylalkyl and the like) gives the secondary amines 29, which yield the desired amides 28 upon coupling to suitable acids using BOP reagent or any other suitable coupling agent.

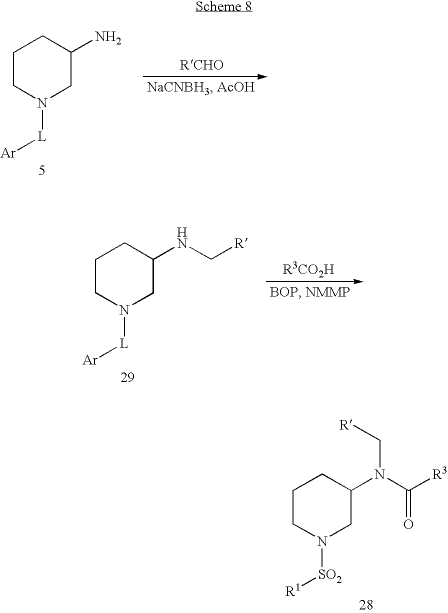

Alternatively, the same series of N-(piperidin-3-yl)carboxamides of formula 30 can be prepared by the method outlined in Scheme 9 wherein X is a leaving group such as halo. An Alkyl group R² can be directly introduced to the N-atom of the amides 4 to form the desired amides 30 under the conditions of phase transfer catalysis by using a suitable catalyst such as tributylammonium bromide.

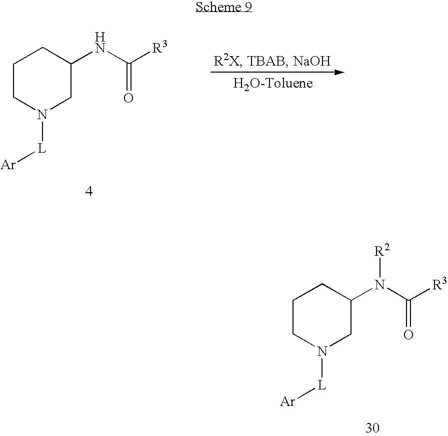

A series of carboxamides of formula 31, wherein A is S, O, CH₂ or NR (R is alkyl, cycloalkyl, arylalkyl, etc.), can be prepared according to the method outlined in Scheme 10, wherein R can be alkyl, aryl, arylalkyl, or the like and X is a leaving group such as halo. Treatment of an ester compound 32 with excess of an alkyl bromide or iodide in the presence of a suitable base such as sodium hydride or LDA and in a suitable solvent such as DMF or THF provides an R-substituted ester 33, which upon basic hydrolysis yields a carboxylic acid 34. Coupling of the carboxylic acid 34 to the 3-aminopiperidine 5 affords the desired product 31.

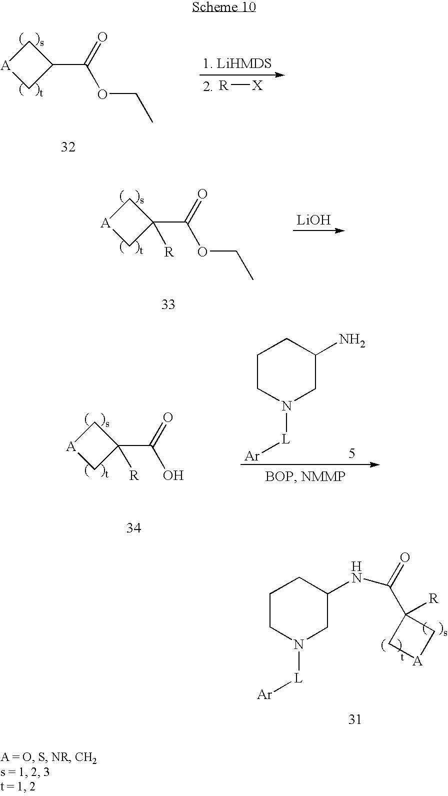

A series of carboxylic acids of formula 38 wherein X is S or O can be prepared according to the method outlined in Scheme 11, wherein R can be alkyl or arylalkyl and Cy can be aryl, heteroaryl, cycloalkyl or heterocylcloalky. Reaction of an appropriate thiol or alcohol 35 with methyl bromoacetate in the presence of a suitable base such as potassium or sodium carbonate, triethylamine or sodium hydride in a suitable solvent such as tetrahydrofuran, acetonitrile or dichloromethane provides a thioether or ether compound 36. Treatment of compound 36 with excess of an alkyl bromide or iodide in the presence of a suitable base such as sodium hydride or LDA and in a suitable solvent such as DMF or THF provides a substituted ester compound 37, which upon basic hydrolysis yield the desired carboxylic acids 38.

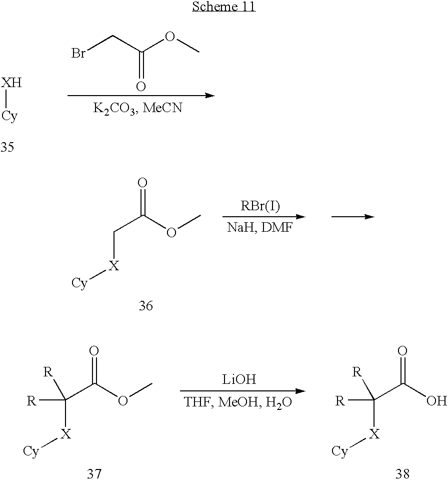

As shown in Scheme 12, alkylation of an ether or thioether 36 with one equivalent of the appropriate alkyl bromide or iodide R′Br(I) in the presence of a suitable base such as NaH, LDA or LiHMDS in a suitable solvent such as DMF or THF, followed by a second alkylation with R″Br(I) in the presence of a suitable base such as NaH and a suitable solvent such as DMSO provides a ester compound 39, which upon basic hydrolysis yields the desired carboxylic acid 40.

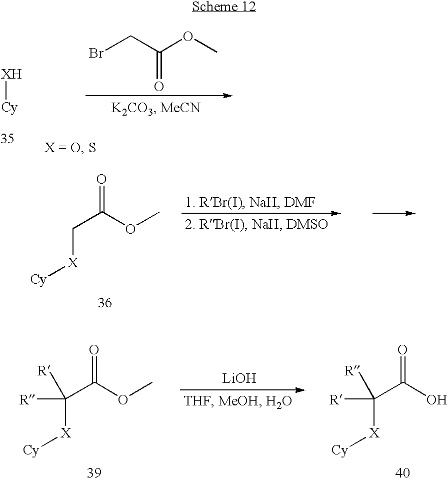

Alternatively, starting with an appropriate cyclic ketone or thioketone 41 and following Scheme 13, a series of carboxylic acids of formula 44 can be prepared wherein the ring in 44 can be non-aromatic, aromatic or heteroaromtic.

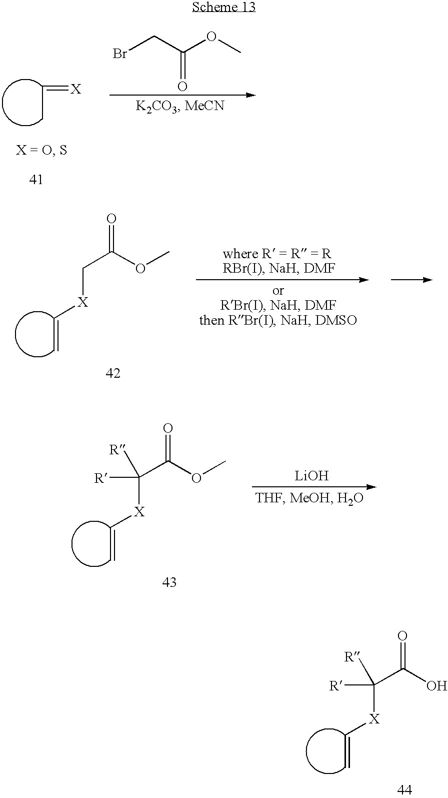

A series of carboxylic acids of formula 49, wherein X=O, S can be prepared by the method outlined in Scheme 14. O- or S-alkylation of compounds 45 with a suitable alkyl chloride or alkyl bromide provides methyl esters 46. Alkylation of 46 with an appropriate alkyl bromide or iodide in the presence of a suitable base such as LDA and in a suitable solvent such as THF yields methyl esters 47, which can undergo a second alkylation with another alkyl bromide or iodide in the presence of a suitable base such as NaH and in a suitable solvent such as DMSO to provide the corresponding esters 48. Finally, basic hydrolysis of esters 48 yields the desired carboxylic acids 49.

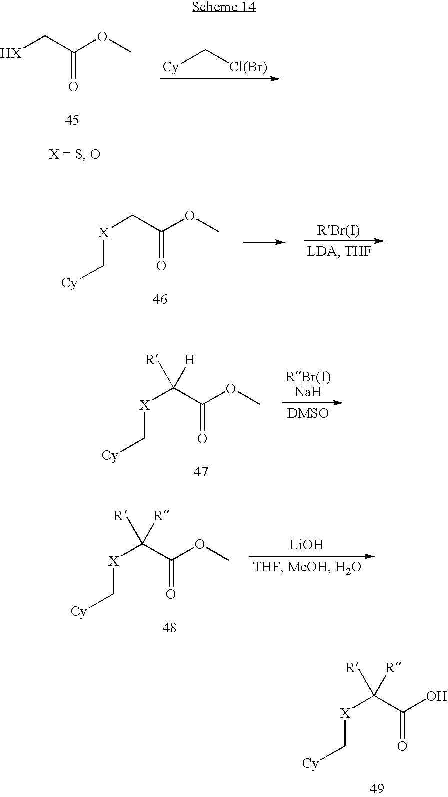

Alternatively, a series of carboxylic acids of formula 53 (wherein X is O, S and u is 1 or 2), can be prepared according to Scheme 15. Reaction of an appropriate alcohol or thiol 50 with chloroacetonitrile in the presence of a suitable base such as sodium ethoxide under suitable conditions such as refluxing provides nitriles 51. Alkylation(s) of 51 in the standard fashion as depicted in Scheme 15 provides nitriles 52, which upon basic hydrolysis provide the desired carboxylic acids 53, wherein Cy can be aryl, heteroaryl, cycloalkyl or heterocylcloalky and the like.

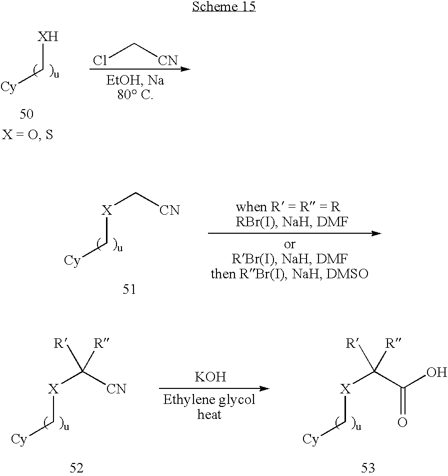

Alternatively, carboxylic acids 59 wherein Cy can be aryl, heteroaryl, cycloalkyl or heterocylcloalky can be prepared by the reaction of an appropriate alcohol CyCH₂OH with thioglycolic acid 54 in the presence of a Lewis acid such as zinc trifluoromethanesulfonate, under suitable conditions such as refluxing to give an acid compound 55. Then 55 can be processed to give the desired carboxylic acids 59 in the fashion as shown in Scheme 16.

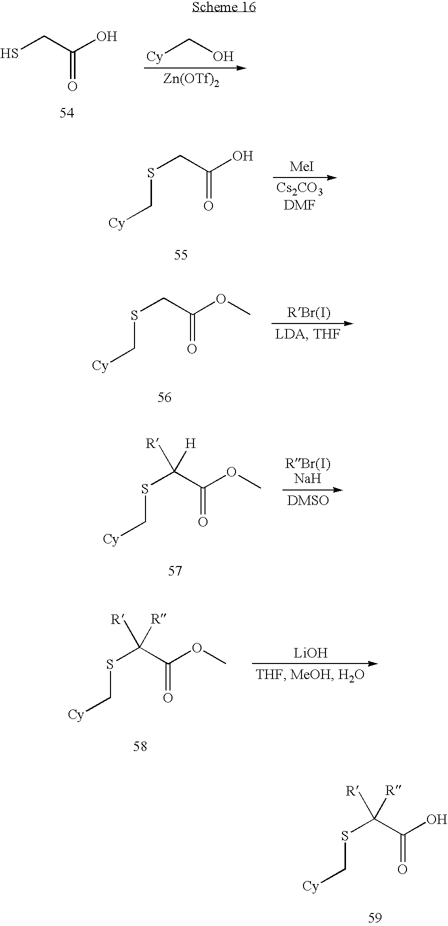

According to scheme 17, a thioether compound 60 can be oxidized to the corresponding sulfone 61 with a suitable oxidant such as 3-chloroperoxybenzoic acid. Following Scheme 17, as previously described, a series of carboxylic acids of formula 63 can be prepared. The same sequence (conversion of the thioether to a sulfone) can be employed in any of the Schemes described earlier.

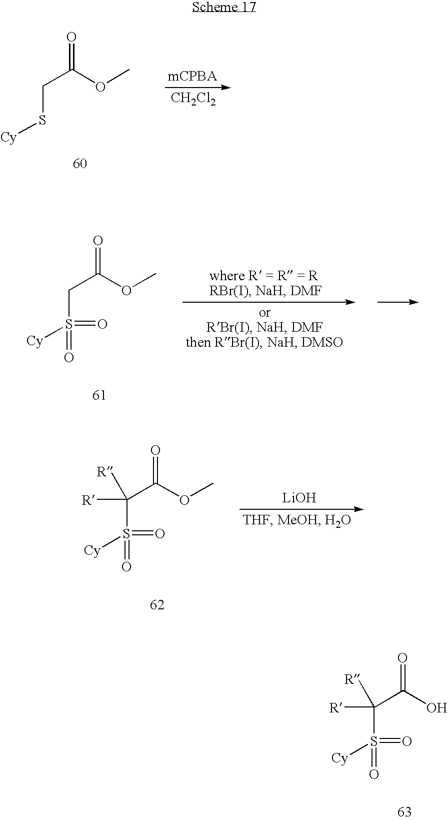

A series of carboxylic acids of formula 68 can be prepared by the method outlined in Scheme 18. An N-Boc glycine methyl ester 64 can undergo C_{α} alkylation in the fashion as shown above to provide an alkylated compound 65. Removal of the Boc group with TFA followed by an N-alkylation with an appropriate alkyl bromide or iodide CyCH₂Br (or I) leads to the formation of an ester 67, which upon basic hydrolysis provides the desired carboxylic acid 68.

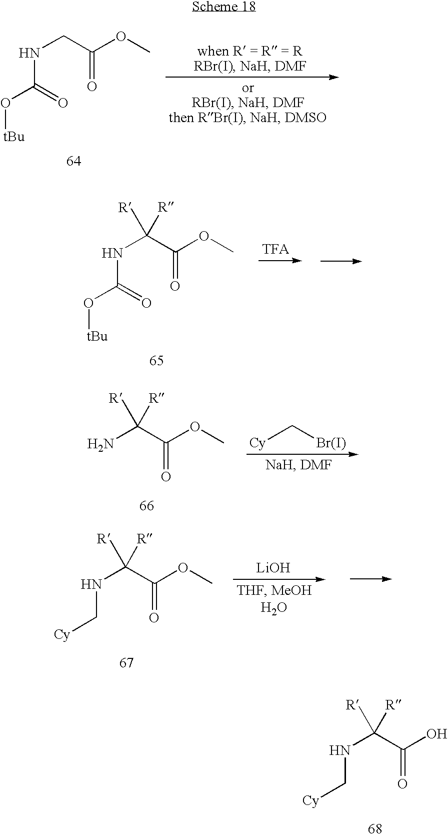

Alternatively, according to Scheme 19, the same series of carboxylic acids of formula 68 can be prepared in a similar fashion as described above, except employing a reductive amination to afford the compound 67 with a corresponding aldehyde CyCHO and a compound 66 under suitable conditions.

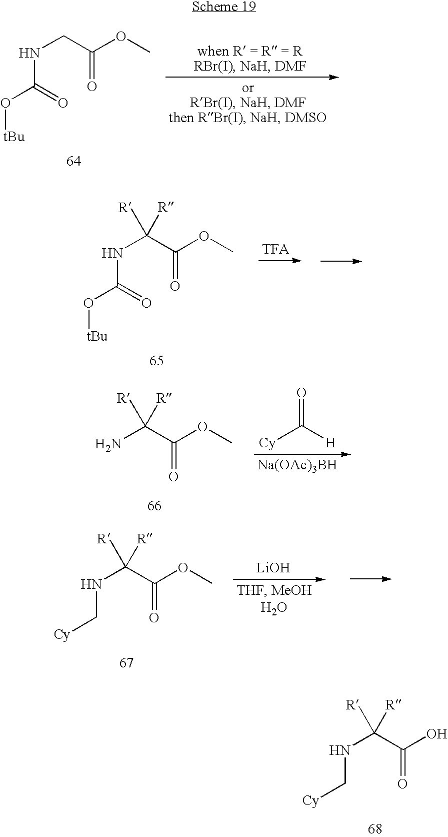

A series of carboxylic acids of formula 72 can be prepared by the method outlined in Scheme 20. Reaction of Cbz-protected amine 69 with 2-bromo methyl acetate provides methyl esters 70. Alkylation(s) in the fashion as shown below provides di-alkylated methyl esters 71. Then, basic hydrolysis of the esters 71 yields the desired carboxylic acids 72. The Cbz group of the compounds 72 can be removed under hydrogenolysis conditions at a later stage.

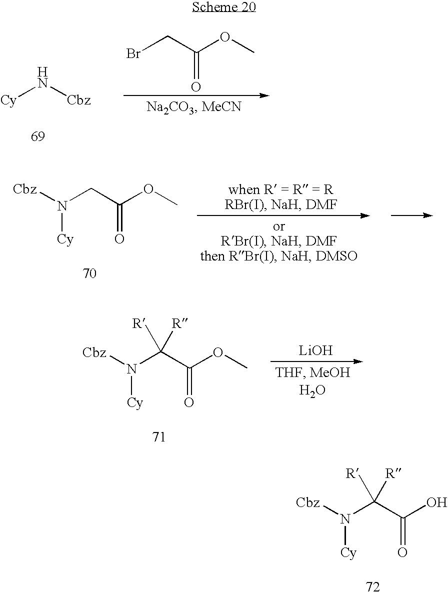

A series of amido compounds of formula 76 can be prepared by the method outlined in Scheme 21. tert-Butyl piperidin-3-ylcarbamate 69 can be coupled to an aryl halide or a heteroaryl halide ArX (wherein Ar can be optionally substituted with one or more substituents such as halo or alkyl) such as bromobenzene in a solvent such as dimethyl sulfoxide, in the presence of a base such as tert-butoxide, to afford a compound of formula 74. The Boc protecting group of 74 can be removed by HCl in 1,4-dioxane to afford an amine compound 75 as an HCl salt. The amine compound 75 can be coupled with a suitable carboxylic acid R³COOH in a suitable solvent such as DMF, in the presence of a suitable base such as 4-methylmorpholine, and in the presence of a suitable coupling reagent such as benzotriazol-1-yloxytris(dimethylamino)phosphonium hexafluorophosphate, to give the final amido compounds of formula 76. 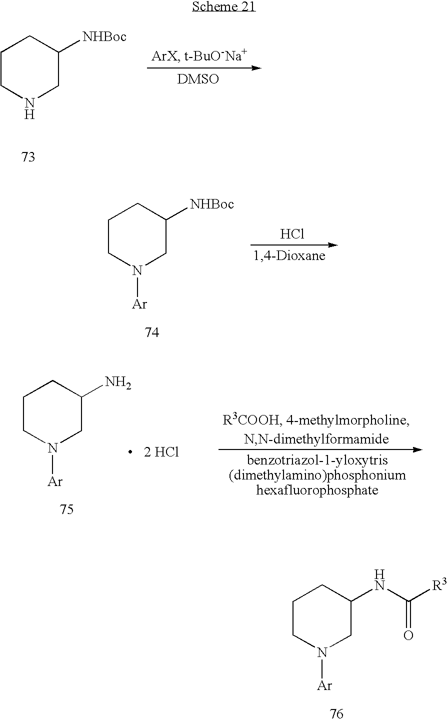
Methods

Compounds of the invention can modulate activity of 11βHSD1 and/or MR. The term “modulate” is meant to refer to an ability to increase or decrease activity of an enzyme or receptor. Accordingly, compounds of the invention can be used in methods of modulating 11βHSD1 and/or MR by contacting the enzyme or receptor with any one or more of the compounds or compositions described herein. In some embodiments, compounds of the present invention can act as inhibitors of 11βHSD1 and/or MR. In further embodiments, the compounds of the invention can be used to modulate activity of 11βHSD1 and/or MR in an individual in need of modulation of the enzyme or receptor by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 and/or MR in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme or receptor. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, hypertension, hyperlipidemia, cognitive impairment, dementia, glaucoma, cardiovascular disorders, osteoporosis, and inflammation. Further examples of 11βHSD1-associated diseases include metabolic syndrome, type 2 diabetes, androgen excess (hirsutism, menstrual irregularity, hyperandrogenism) and polycystic ovary syndrome (PCOS).

The present invention further provides methods of modulating MR activity by contacting the MR with a compound of the invention, pharmaceutically acceptable salt, prodrug, or composition thereof. In some embodiments, the modulation can be inhibition. In further embodiments, methods of inhibiting aldosterone binding to the MR (optionally in a cell) are provided. Methods of measuring MR activity and inhibition of aldosterone binding are routine in the art.

The present invention further provides methods of treating a disease associated with activity or expression of the MR. Examples of diseases associated with activity or expression of the MR include, but are not limited to hypertension, as well as cardiovascular, renal, and inflammatory pathologies such as heart failure, atherosclerosis, arteriosclerosis, coronary artery disease, thrombosis, angina, peripheral vascular disease, vascular wall damage, stroke, dyslipidemia, hyperlipoproteinaemia, diabetic dyslipidemia, mixed dyslipidemia, hypercholesterolemia, hypertriglyceridemia, and those associated with type 1 diabetes, type 2 diabetes, obesity metabolic syndrome, insulin resistance and general aldosterone-related target organ damage.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease (non-limiting examples are preventing metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS);

(2) inhibiting the disease; for example, inhibiting a disease, condition or disorder in an individual who is experiencing or displaying the pathology or symptomatology of the disease, condition or disorder (i.e., arresting further development of the pathology and/or symptomatology) such as inhibiting the development of metabolic syndrome, hypertension, obesity, insulin resistance, hyperglycemia, hyperlipidemia, type 2 diabetes, androgen excess (hirsutism, menstrual irregularity, hyperandrogenism) or polycystic ovary syndrome (PCOS), stabilizing viral load in the case of a viral infection; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS), or lowering viral load in the case of a viral infection.

Pharmaceutical Formulations and Dosage Forms

When employed as pharmaceuticals, the compounds of Formula I can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to radio-labeled compounds of the invention that would be useful not only in radio-imaging but also in assays, both in vitro and in vivo, for localizing and quantitating the enzyme in tissue samples, including human, and for identifying ligands by inhibition binding of a radio-labeled compound. Accordingly, the present invention includes enzyme assays that contain such radio-labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I , ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I , ³⁵S and ⁸²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the enzyme. Accordingly, the ability of a test compound to compete with the radio-labeled compound for binding to the enzyme directly correlates to its binding affinity.

Kits

The present invention also includes pharmaceutical kits useful, for example, in the treatment or prevention of 11βHSD1- or MR-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compound of the Examples were found to inhibitors of 11βHSD1 and/or MR according to one or more of the assays provided herein.

### EXAMPLES

### Example 1

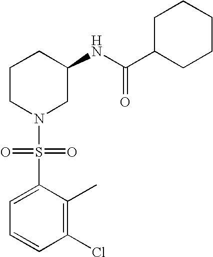

### N-(3R)-1-[(3-Chloro-2-methylphenyl)sulfonyl]piperidin-3-ylcyclohexanecarboxamide

Step 1: N-[(3R)-piperidin-3-yl]cyclohexanecarboxamide hydrochloride

Cyclohexanecarbonyl chloride (70.0 μL, 0.515 mmol) was added to a mixture of tert-butyl (3R)-3-aminopiperidine-1-carboxylate (100.0 mg, 0.499 mmol) and potassium carbonate (150 mg, 2.1 eq.) in acetonitrile (3.0 mL) at RT. The reaction mixture was stirred at RT for 1 h, and was filtered. The filtrate was concentrated under reduced pressure. The residue was treated with 4.0 M of hydrogen chloride in 1,4-Dioxane (2.0 mL) at RT for 1 h. The solvent was evaporated under reduced pressure to give the product which was directly used in next step reaction without further purification.

Step 2: N-(3R)-1-[(3-chloro-2-methylphenyl)sulfonyl]piperidin-3-ylcyclohexanecarboxamid

N-[(3R)-piperidin-3-yl]cyclohexanecarboxamide hydrochloride (12.3 mg, 50.0 μmol) in acetonitrile (0.8 mL) was treated diisopropylethylamine (20.0 μL). To the solution was added 3-chloro-2-methylbenzenelsulfonyl chloride (11.3 mg, 50.0 μmol). The resulting mixture was stirred at RT for overnight, and then was adjusted to PH=2.0 with TFA. The mixture was diluted with DMSO (1.0 mL), and was purified by prep-HPLC to give the desired product N-(3R)-1-[(3-chloro-2-methylphenyl)sulfonyl]piperidin-3-yl-cyclohexanecarboxamide. LCMS: (M+H)⁺=399.0/401.0.

### Example 2

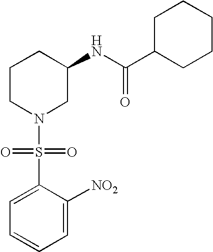

### N-(3R)-1-[(2-Nitrophenyl)sulfonyl]piperidin-3-ylcyclohexanecarboxamide

This compound was prepared using procedures analogous to those for example 1. LCMS: (M+H)⁺=396.0.

### Example 3

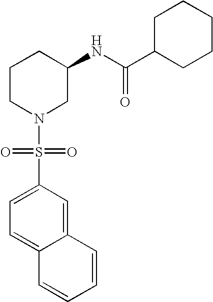

### N-[(3R)-1-(2-Naphthylsulfonyl)piperidin-3-yl]cyclohexanecarboxamide

This compound was prepared using procedures analogous to those for example 1. LCMS: (M+H)⁺=401.1.

### Example 4

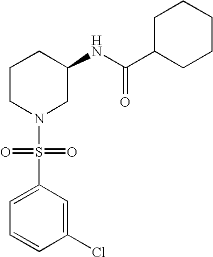

### N-(3R)-1-[(3-chlorophenyl)sulfonyl]piperidin-3-ylcyclohexanecarboxamide

This compound was prepared using procedures analogous to those for example 1. LCMS: (M+H)⁺=385.1/387.1.

### Example 5

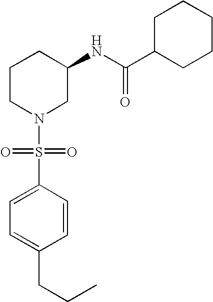

### N-(3R)-1-[(4-propylphenyl)sulfonyl]piperidin-3-ylcyclohexanecarboxamide

This compound was prepared using procedures analogous to those for example 1. LCMS: (M+H)⁺=393.1.

### Example 6

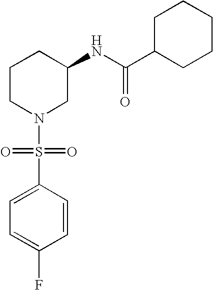

### N-{(3R)-1-[(4-fluorophenyl)sulfonyl]piperidin-3-yl}cyclohexanecarboxamide

This compound was prepared using procedures analogous to those for example 1. LCMS: (M+H)⁺=369.1.

### Example 7

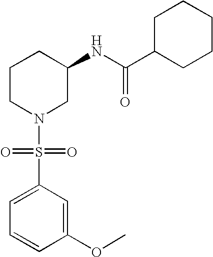

### N-{(3R)-1-[(3-methoxyphenyl)sulfonyl]piperidin-3-yl}cyclohexanecarboxamide

This compound was prepared using procedures analogous to those for example 1. LCMS: (M+H)⁺=381.1.

### Example 8

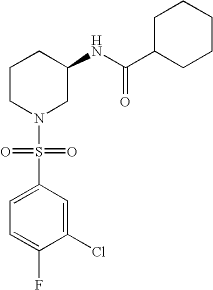

### N-(3R)-1-[(3-chloro-4-fluorophenyl)sulfonyl]piperidin-3-ylcyclohexanecarboxamide

This compound was prepared using procedures analogous to those for example 1. LCMS: (M+H)⁺=403.0/405.0.

### Example 9

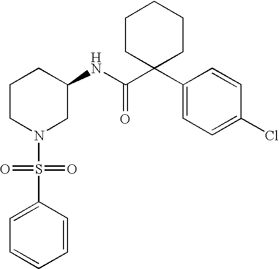

### 1-(4-Chlorophenyl)-N-[(3R)-1-(phenylsulfonyl)piperidin-3-yl]cyclohexanecarboxamide

Step 1: tert-Butyl (3R)-3-([1-(4-chlorophenyl)cyclohexyl]carbonylamino)piperidine-1-carboxylate

To a mixture of 1-(4-chlorophenyl)cyclohexanecarboxylic acid (24.6 mg, 103 μmol) and tert-butyl (3R)-3-aminopiperidine-1-carboxylate (20.0 mg, 99.7 μmol) in N,N-Dimethylformamide (1.00 mL) was added benzotriazol-1-yloxytris(dimethylamino)phosphonium hexafluorophosphate (44.2 mg, 99.9 μmol), followed by and 4-methylmorpholine (50.0 μL). The mixture was stirred at rt for overnight. The mixture was diluted with ethyl acetate (5 mL) and washed with NaHCO₃ (7.5%, 3×1 mL) and brine (1 mL). The organic layer was dried over Na₂SO₄, filtered, concentrated under reduced pressure to give the product which was directly used in next step reaction without further purification.

Step 2: 1-(4-Chlorophenyl)-N-[(3R)-piperidin-3-yl]cyclohexanecarboxamide hydrochloride

The tert-butyl (3R)-3-([1-(4-chlorophenyl)cyclohexyl]carbonylamino)-piperidine-1-carboxylate was treated with 4.0 M of hydrogen chloride in 1,4-dioxane (0.5 mL) at RT for 1 h. The solvent was evaporated to give the corresponding product which was directly used in next step reaction without further purification.

Step 3: 1-(4-Chlorophenyl)-N-[(3R)-1-(phenylsulfonyl)piperidin-3-yl]cyclohexanecarboxamide

The 1-(4-chlorophenyl)-N-[(3R)-piperidin-3-yl]-cyclohexanecarboxamide hydrochloride (50 μmol) in acetonitrile (1.0 mL) was treated with N,N-diisopropylethylamine (20.0 μL) at RT, then benzenesulfonyl chloride (9.27 mg, 52.5 μmol) was added. The reaction mixture was stirred at RT for overnight, and was diluted with DMSO (0.8 mL) and adjusted to pH=2.0. The resulting solution was submitted to purify by prep.-HPLC to give the corresponding desired product 1-(4-chlorophenyl)-N-[(3R)-1-(phenylsulfonyl)piperidin-3-yl]cyclohexanecarboxamid. LCMS: (M+H)⁺=461.1/463.1.

### Example 10

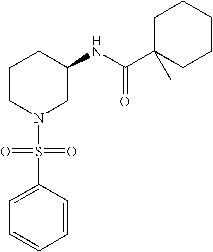

### 1-Methyl-N-[(3R)-1-(phenylsulfonyl)piperidin-3-yl]cyclohexanecarboxamide

This compound was prepared using procedures analogous to those for example 9. LCMS: (M+H)⁺=365.2.

### Example 11

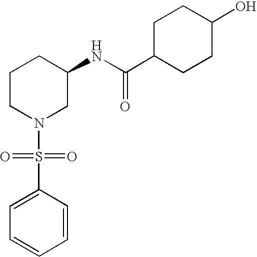

### 4-Hydroxy-N-[(3R)-1-(phenylsulfonyl)piperidin-3-yl]cyclohexanecarboxamide

This compound was prepared using procedures analogous to those for example 9. LCMS: (M+H)⁺=367.0.

### Example 12

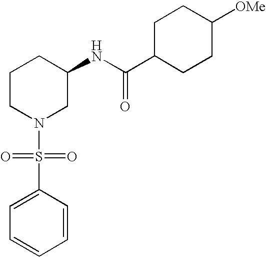

### 4-Methoxy-N-[(3R)-1-(phenylsulfonyl)piperidin-3-yl]cyclohexanecarboxamide

This compound was prepared using procedures analogous to those for example 9. LCMS: (M+H)⁺=381.0.

### Example 13

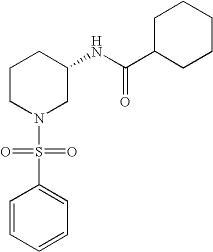

### N-[(3S)-1-(phenylsulfonyl)piperidin-3-yl]cyclohexanecarboxamide

This compound was prepared using procedures analogous to those for example 1. LCMS: (M+H)⁺=351.1.

### Example 14

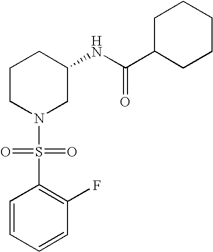

### N-{(3S)-1-[(2-fluorophenyl)sulfonyl]piperidin-3-yl}cyclohexanecarboxamide

This compound was prepared using procedures analogous to those for example 1. LCMS: (M+H)⁺=369.1.

### Example 15

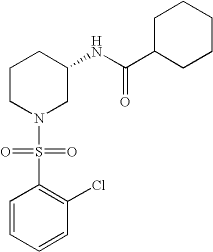

### N-{(3S)-1-[(2-Chlorophenyl)sulfonyl]piperidin-3-yl}cyclohexanecarboxamide

This compound was prepared using procedures analogous to those for example 1. LCMS: (M+H)⁺=385.1/387.1.

### Example 16

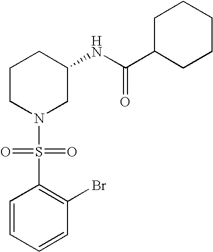

### N-{(3S)-1-[(2-Bromrophenyl)sulfonyl]piperidin-3-yl}cyclohexanecarboxamide

This compound was prepared using procedures analogous to those for example 1. LCMS: (M+H)⁺=429.0/431.0.

### Example 17

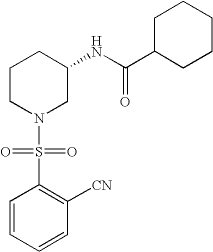

### N-{(3S)-1-[(2-Cyanophenyl)sulfonyl]piperidin-3-yl}cyclohexanecarboxamide

This compound was prepared using procedures analogous to those for example 1. LCMS: (M+H)⁺=376.1.

### Example 18

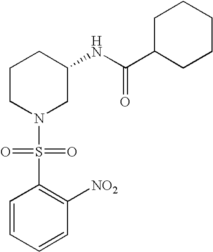

### N-{(3S)-1-[(2-Nitrophenyl)sulfonyl]piperidin-3-yl}cyclohexanecarboxamide

This compound was prepared using procedures analogous to those for example 1. LCMS: (M+H)⁺=396.1.

### Example 19

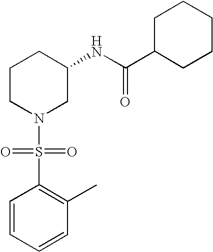

### N-{(3S)-1-[(2-methylphenyl)sulfonyl]piperidin-3-yl}cyclohexanecarboxamide

This compound was prepared using procedures analogous to those for example 1. LCMS: (M+H)⁺=365.1.

### Example 20

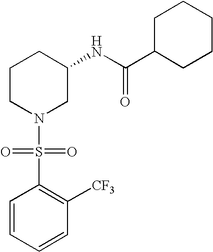

### N-((3S)-1-{[2-(trifluoromethyl)phenyl]sulfonyl}piperidin-3-yl)cyclohexanecarboxamide

This compound was prepared using procedures analogous to those for example 1. LCMS: (M+H)⁺=419.1.

### Example 21

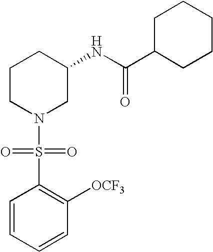

### N-((3S)-1-{[2-(Trifluoromethoxy)phenyl]sulfonyl}piperidin-3-yl)cyclohexanecarboxamide

This compound was prepared using procedures analogous to those for example 1. LCMS: (M+H)⁺=435.1.

### Example 22

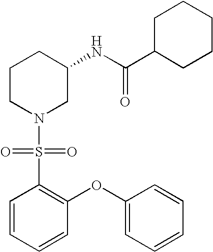

### N-{(3S)-1-[(2-Phenoxyphenyl)sulfonyl]piperidin-3-yl}cyclohexanecarboxamide

This compound was prepared using procedures analogous to those for example 1. LCMS: (M+H)⁺=443.1.

### Example 23

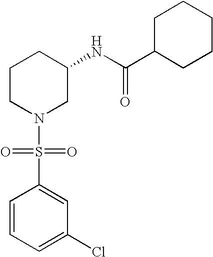

### N-{(3S)-1-[(3-Chlorophenyl)sulfonyl]piperidin-3-yl}cyclohexanecarboxamide

This compound was prepared using procedures analogous to those for example 1. LCMS: (M+H)⁺=385.1/387.0.

### Example 24

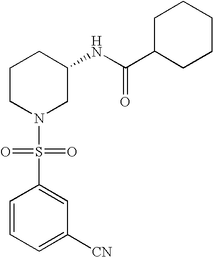

### N-{(3S)-1-[(3-Cyanophenyl)sulfonyl]piperidin-3-yl}cyclohexanecarboxamide

This compound was prepared using procedures analogous to those for example 1. LCMS: (M+H)⁺=376.1.

### Example 25

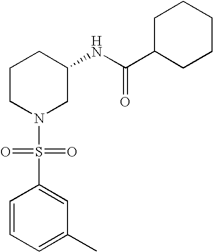

### N-{(3S)-1-[(3-Methylphenyl)sulfonyl]piperidin-3-yl}cyclohexanecarboxamide

This compound was prepared using procedures analogous to those for example 1. LCMS: (M+H)⁺=365.1.

### Example 26

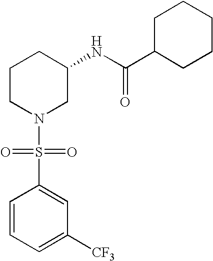

### N-((3S)-1-{[3-(Trifluoromethyl)phenyl}sulfonyl piperidin-3-yl)cyclohexanecarboxamide

This compound was prepared using procedures analogous to those for example 1. LCMS: (M+H)⁺=419.1.

### Example 27

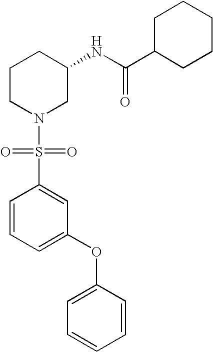

### N-{(3S)-1-[(3-Phenoxyphenyl)sulfonyl]piperidin-3-yl}cyclohexanecarboxamide

This compound was prepared using procedures analogous to those for example 1. LCMS: (M+H)⁺=443.1.

### Example 28

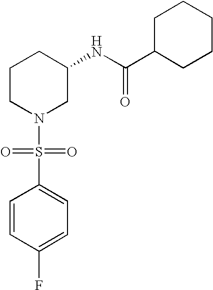

### N-{(3S)-1-[(4-fluorophenyl)sulfonyl]piperidin-3-yl}cyclohexanecarboxamide

This compound was prepared using procedures analogous to those for example 1. LCMS: (M+H)⁺=369.1.

### Example 29

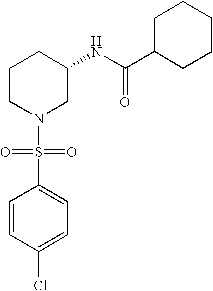

### N-{(3S)-1-[(4-chlorophenyl)sulfonyl]piperidin-3-yl}cyclohexanecarboxamide

This compound was prepared using procedures analogous to those for example 1. LCMS: (M+H)⁺=385.1/387.1.

### Example 30

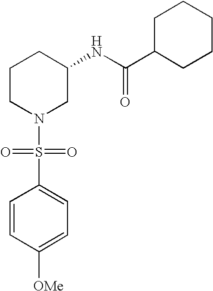

### N-{(3S)-1-[(4-methoxyphenyl)sulfonyl]piperidin-3-yl}cyclohexanecarboxamide

This compound was prepared using procedures analogous to those for example 1. LCMS: (M+H)⁺=381.1.

### Example 31

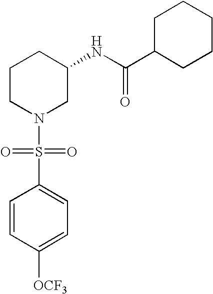

### N-((3S)-1-{[4-(trifluoromethoxy)phenyl]sulfonyl}piperidin-3-yl)-cyclohexane-carboxamide

This compound was prepared using procedures analogous to those for example 1. LCMS: (M+H)⁺=435.1.

### Example 32

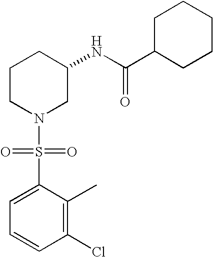

### N-(3S)-1-[(3-Chloro-2-methylphenyl)sulfonyl]piperidin-3-ylcyclohexanecarboxamide

This compound was prepared using procedures analogous to those for example 1. LCMS: (M+H)⁺=399.1/401.1.

### Example 33

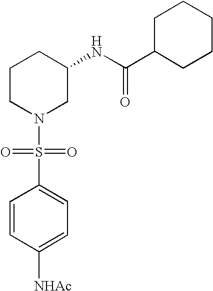

### N-((3S)-1-{[4-(acetylamino)phenyl]sulfonyl}piperidin-3-yl)cyclohexanecarboxamide

This compound was prepared using procedures analogous to those for example 1. LCMS: (M+H)⁺=408.1.

### Example 34

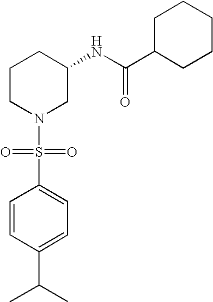

### N-{(3S)-1-[(4-isopropylphenyl)sulfonyl]piperidin-3-yl}cyclohexanecarboxamide

This compound was prepared using procedures analogous to those for example 1. LCMS: (M+H)⁺=393.2.

### Example 35

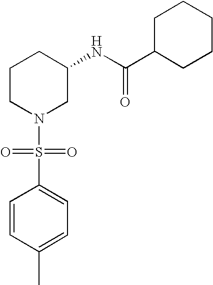

### N-{(3S)-1-[(4-methylphenyl)sulfonyl]piperidin-3-yl}cyclohexanecarboxamide

This compound was prepared using procedures analogous to those for example 1. LCMS: (M+H)⁺=365.1.

### Example 36

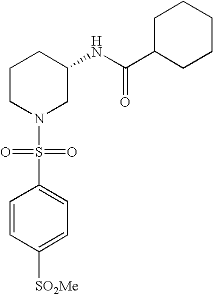

### N-((3S)-1-{[4-(methylsulfonyl)phenyl]sulfonyl}piperidin-3-yl)cyclohexanecarboxamide

This compound was prepared using procedures analogous to those for example 1. LCMS: (M+H)⁺=429.1.

### Example 37

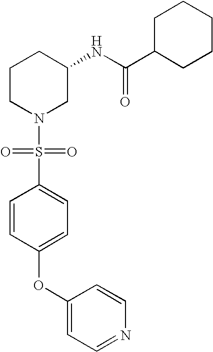

### N-((3S)-1-{[4-(pyridin-4-yloxy)phenyl]sulfonyl}piperidin-3-yl)cyclohexanecarboxamide

This compound was prepared using procedures analogous to those for example 1. LCMS: (M+H)⁺=444.1.

### Example 38

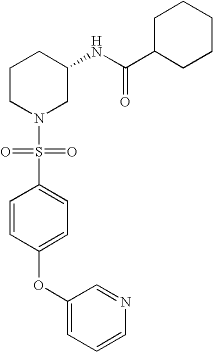

### N-((3S)-1-{[4-(pyridin-3-yloxy)phenyl]sulfonyl}piperidin-3-yl)cyclohexanecarboxamide

This compound was prepared using procedures analogous to those for example 1. LCMS: (M+H)⁺=444.1.

### Example 39

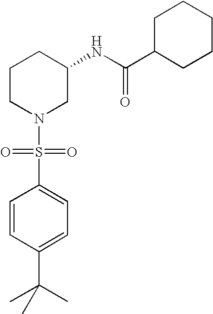

### N-{(3S)-1-[(4-tert-butylphenyl)sulfonyl]piperidin-3-yl}cyclohexanecarboxamide

This compound was prepared using procedures analogous to those for example 1. LCMS: (M+H)⁺=407.2.

### Example 40

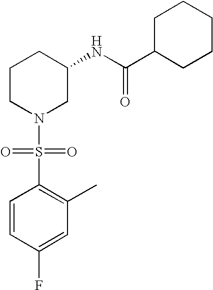

### N-{(3S)-1-[(4-fluoro-2-methylphenyl)sulfonyl]piperidin-3-yl}cyclohexanecarboxamide

This compound was prepared using procedures analogous to those for example 1. LCMS: (M+H)⁺=383.1.

### Example 41

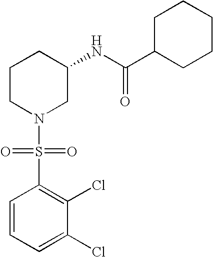

### N-{(3S)-1-[(2,3-dichlorophenyl)sulfonyl]piperidin-3-yl}cyclohexanecarboxamide

This compound was prepared using procedures analogous to those for example 1. LCMS: (M+H)⁺=419.0/421.0.

### Example 42

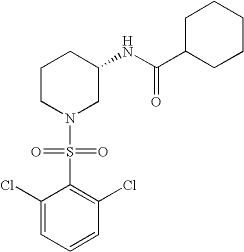

### N-{(3S)-1-[(2,6-dichlorophenyl)sulfonyl]piperidin-3-yl}cyclohexanecarboxamide

This compound was prepared using procedures analogous to those for example 1. LCMS: (M+H)⁺=419.0/421.1.

### Example 43

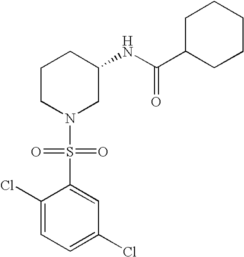

### N-{(3S)-1-[(2,5-dichlorophenyl)sulfonyl]piperidin-3-yl}cyclohexanecarboxamide

This compound was prepared using procedures analogous to those for example 1. LCMS: (M+H)⁺=419.1/421.0.

### Example 44

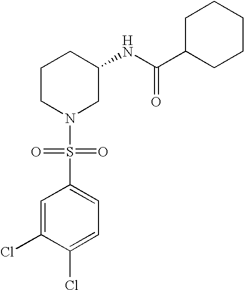

### N-{(3S)-1-[(3,4-dichlorophenyl)sulfonyl]piperidin-3-yl}cyclohexanecarboxamide

This compound was prepared using procedures analogous to those for example 1. LCMS: (M+H)⁺=419.0/421.0.

### Example 45

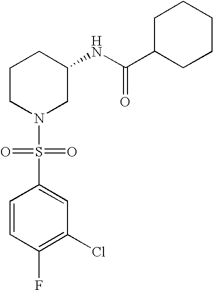

### N-{(3S)-1-[(3-chloro-4-fluorophenyl)sulfonyl]piperidin-3-yl}cyclohexanecarboxamide

This compound was prepared using procedures analogous to those for example 1. LCMS: (M+H)⁺=403.1/405.1.

### Example 46

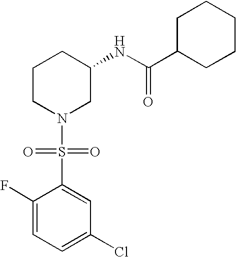

### N-{(3S)-1-[(5-chloro-2-fluorophenyl)sulfonyl]piperidin-3-yl}cyclohexanecarboxamide

This compound was prepared using procedures analogous to those for example 1. LCMS: (M+H)⁺=403.1/405.1.

### Example 47

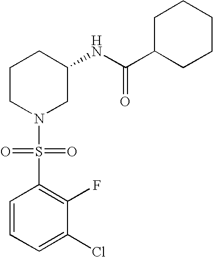

### N-{(3S)-1-[(3-chloro-2-fluorophenyl)sulfonyl]piperidin-3-yl}cyclohexanecarboxamide

This compound was prepared using procedures analogous to those for example 1. LCMS: (M+H)⁺=403.0/405.1.

### Example 48

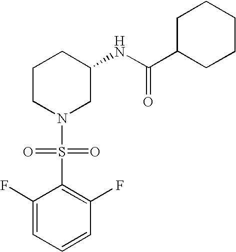

### N-{(3S)-1-[(2,6-difluorophenyl)sulfonyl]piperidin-3-yl}cyclohexanecarboxamide

This compound was prepared using procedures analogous to those for example 1. LCMS: (M+H)⁺=387.1.

### Example 49

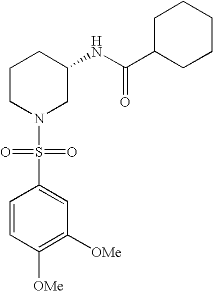

### N-{(3S)-1-[(3,4-dimethoxyphenyl)sulfonyl]piperidin-3-yl}cyclohexanecarboxamide

This compound was prepared using procedures analogous to those for example 1. LCMS: (M+H)⁺=411.1.

### Example 50

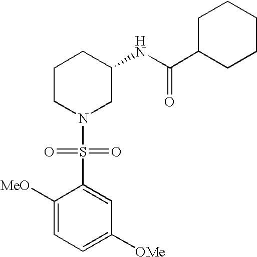

### N-{(3S)-1-[(2,5-dimethoxyphenyl)sulfonyl]piperidin-3-yl}cyclohexanecarboxamide

This compound was prepared using procedures analogous to those for example 1. LCMS: (M+H)⁺=411.1.

### Example 51

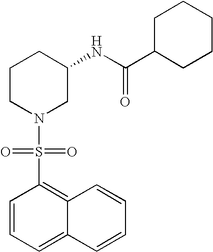

### N-[(3S)-1-(1-naphthylsulfonyl)piperidin-3-yl]cyclohexanecarboxamide

This compound was prepared using procedures analogous to those for example 1. LCMS: (M+H)⁺=401.1.

### Example 52

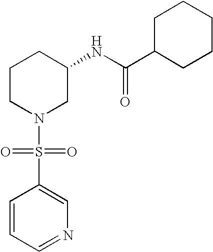

### N-[(3S)-1-(pyridin-3-ylsulfonyl)piperidin-3-yl]cyclohexanecarboxamide

This compound was prepared using procedures analogous to those for example 1. LCMS: (M+H)⁺=352.1.

### Example 53

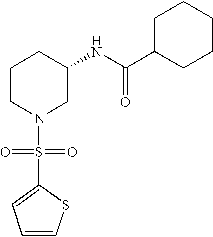

### N-[(3S)-1-(2-thienylsulfonyl)piperidin-3-yl]cyclohexanecarboxamide

This compound was prepared using procedures analogous to those for example 7. LCMS: (M+H)⁺=357.1.

### Example 54

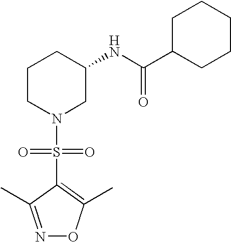

### N-{(3S)-1-[(3,5-dimethylisoxazol-4-yl)sulfonyl]piperidin-3-yl}cyclohexanecarboxamide

This compound was prepared using procedures analogous to those for example 1. LCMS: (M+H)⁺=370.1.

### Example 55

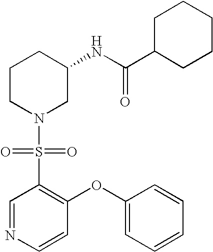

### N-{(3S)-1-[(4-Phenoxypyridin-3-yl)sulfonyl]piperidin-3-yl}cyclohexanecarboxamide

This compound was prepared using procedures analogous to those for example 1. LCMS: (M+H)⁺=444.1.

### Example 56

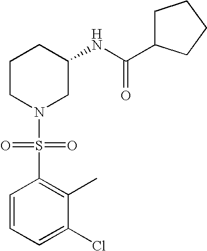

### N-{(3S)-1-[(3-Chloro-2-methylphenyl)sulfonyl]piperidin-3-yl}cyclopentanecarboxamide

Step 1: (3S)-1-[(3-chloro-2-methylphenyl)sulfonyl]piperidin-3-amine hydrochloride

3-Chloro-2-methylbenzenesulfonyl chloride (455 mg, 2.02 mmol) was added to a mixture of tert-butyl (3S)-piperidin-3-ylcarbamate (400.0 mg, 2.00 mmol) and N,N-diisopropylethylamine (355 μL, 204 mmol) in acetonitrile (5.0 mL) at 0° C. The ice-water bath was removed after 10 min, and the mixture was stirred at RT for overnight. The solvent was evaporated. The residue was treated with 4.0 M of hydrogen chloride in 1,4-dioxane (3.0 mL) at RT for 1 h. The solvent was removed under reduced pressure to give the product which was directly used in next step reaction without further purification.

Step 2: N-{(3S)-1-[(3-chloro-2-methylphenyl)sulfonyl]piperidin-3-yl}cyclopentanecarboxamide

(3S)-1-[(3-Chloro-2-methylphenyl)sulfonyl]piperidin-3-amine hydrochloride (50 umol) in Acetonitrile (1.00 mL) was treated with N,N-diisopropylethylamine (20.0 μL, 115 umol). To the resulting solution was added cyclopetanecarbonyl chloride (7.0 mg, 52.5 umol) at RT. The mixture was stirred at RT for 1 h, and was diluted with DMSO (0.8 mL) and adjusted with TFA to pH=2.0. The resulting solution was submitted to purify by prep.-HPLC to give the desired product N-{(3S)-1-[(3-chloro-2-methylphenyl)sulfonyl]piperidin-3-yl}cyclopentanecarboxamide. LCMS: (M+H)⁺=385.1/387.1.

### Example 57

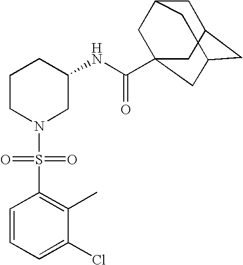

### N-{(3S)-1-[(3-Chloro-2-methylphenyl)sulfonyl]piperidin-3-yl}adamantane-1-carboxamide

This compound was prepared using procedures analogous to those for example 56. LCMS: (M+H)⁺=451.1/453.1.

### Example 58

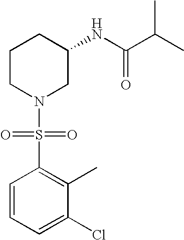

### N-{(3S)-1-[(3-Chloro-2-methylphenyl)sulfonyl]piperidin-3-yl}-2-methylpropanamide

This compound was prepared using procedures analogous to those for example 56. LCMS: (M+H)⁺=359.1/361.0.

### Example 59

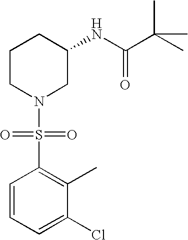

### N-{(3S)-1-[(3-Chloro-2-methylphenyl)sulfonyl]piperidin-3-yl}-2,2-dimethylpropanamide

This compound was prepared using procedures analogous to those for example 56. LCMS: (M+H)⁺=373.1/375.1.

### Example 60

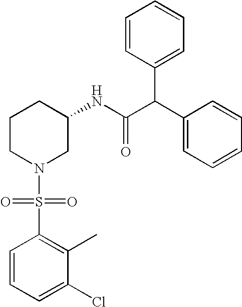

### N-{(3S)-1-[(3-chloro-2-methylphenyl)sulfonyl]piperidin-3-yl}-2,2-diphenylacetamide

This compound was prepared using procedures analogous to those for example 56. LCMS: (M+H)⁺=483.1/485.1.

### Example 61

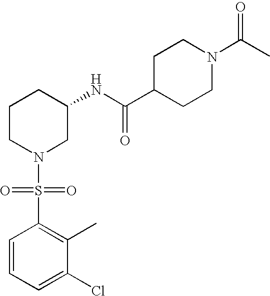

### 1-Acetyl-N-{(3S)-1-[(3-chloro-2-methylphenyl)sulfonyl]piperidin-3-yl}piperidine-4-carboxamide

This compound was prepared using procedures analogous to those for example 56. LCMS: (M+H)⁺=442.1/444.1.

### Example 62

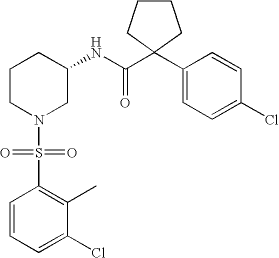

### N-{(3S)-1-[(3-chloro-2-methylphenyl)sulfonyl]piperidin-3-yl}-1-(4-chlorophenyl)cyclopentanecarboxamide

This compound was prepared using procedures analogous to those for example 56. LCMS: (M+H)⁺=495.1/497.1.

### Example 63

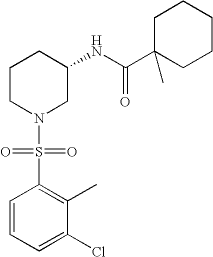

### N-{(3S)-1-[(3-Chloro-2-methylphenyl)sulfonyl]piperidin-3-yl}-1-methylcyclohexanecarboxamide

N-Methyl morpholine (40.0 μL) was added to a mixture of BOP (22.3 mg, 50 μmol), 1-methylcyclohexanecarboxylic acid (7.1 mg, 50 μmol) and (3S)-1-[(3-chloro-2-methylphenyl)sulfonyl]-piperidin-3-amine hydrochloride (50 μmol) in DMF (700 μL) at RT. The mixture was stirred at RT for 3 h, and then was adjusted by TFA to PH=2.0, and diluted with DMSO (1100 μL). The resulting solution was purified by prep.-HPLC to afford the desired product N-{(3S)-1-[(3-chloro-2-methylphenyl)sulfonyl]piperidin-3-yl}-1-methylcyclohexanecarboxamide. LCMS: (M+H)⁺=413.1/415.1.

### Example 64

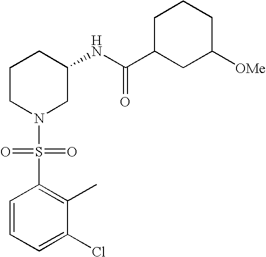

### N-{(3S)-1-[(3-chloro-2-methylphenyl)sulfonyl]piperidin-3-yl}-3-methoxycyclohexanecarboxamide

This compound was prepared using procedures analogous to those for example 56. LCMS: (M+H)⁺=429.1/431.1.

### Example 65

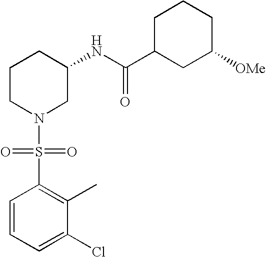

### trans-N-{(3S)-1-[(3-Chloro-2-methylphenyl)sulfonyl]piperidin-3-yl}-3-methoxycyclohexanecarboxamide

This compound was prepared using procedures analogous to those for example 56. LCMS: (M+H)⁺=429.1/431.1.

### Example 66

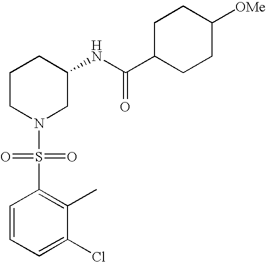

### N-{(3S)-1-[(3-Chloro-2-methylphenyl)sulfonyl]piperidin-3-yl}-4-methoxycyclohexanecarboxamide

This compound was prepared using procedures analogous to those for example 56. LCMS: (M+H)⁺=429.1/431.1.

### Example 67

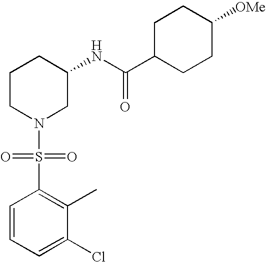

### trans-N-{(3S)-1-[(3-Chloro-2-methylphenyl)sulfonyl]piperidin-3-yl}-4-methoxycyclohexanecarboxamide

This compound was prepared using procedures analogous to those for example 56. LCMS: (M+H)⁺=429.1/431.1.

### Example 68

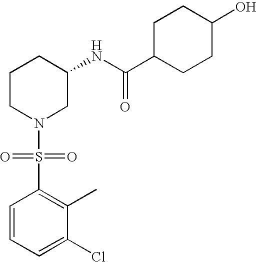

### N-{(3S)-1-[(3-chloro-2-methylphenyl)sulfonyl]piperidin-3-yl}-4-hydroxycyclohexanecarboxamide

This compound was prepared using procedures analogous to those for example 56. LCMS: (M+H)⁺=415.1/417.1.

### Example 69

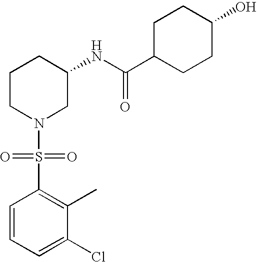

### trans-N-{(3S)-1-[(3-Chloro-2-methylphenyl)sulfonyl]piperidin-3-yl}-4-hydroxycyclohexanecarboxamide

This compound was prepared using procedures analogous to those for example 56. LCMS: (M+H)⁺=415.1/417.1.

### Example 70

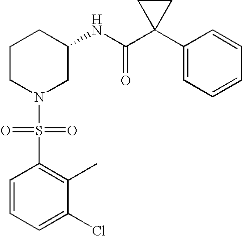

### N-{(3S)-1-[(3-Chloro-2-methylphenyl)sulfonyl]piperidin-3-yl}-1-phenylcyclopropanecarboxamide

This compound was prepared using procedures analogous to those for example 56. LCMS: (M+H)⁺=433.1/435.1.

### Example 71

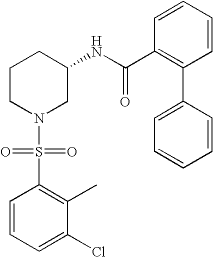

### N-{(3S)-1-[(3-Chloro-2-methylphenyl)sulfonyl]piperidin-3-yl}biphenyl-2-carboxamide

This compound was prepared using procedures analogous to those for example 56. LCMS: (M+H)⁺=469.0/471.1.

### Example 72

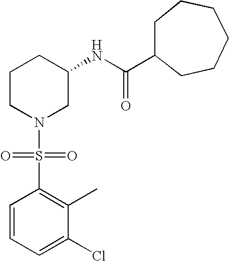

### N-{(3S)-1-[(3-Chloro-2-methylphenyl)sulfonyl]piperidin-3-yl}cycloheptanecarboxamide

This compound was prepared using procedures analogous to those for example 56. LCMS: (M+H)⁺=413.1/415.1.

### Example 73

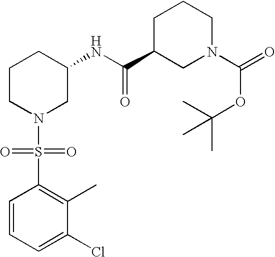

### tert-Butyl (3S)-3-[((3S)-1-[(3-chloro-2-methylphenyl)sulfonyl]piperidin-3-ylamino)carbonyl]piperidine-1-carboxylate

This compound was prepared using procedures analogous to those for example 56. LCMS: (M+Na)⁺=522.1/524.1; (M-56)⁺=444.1/446.1.

### Example 74

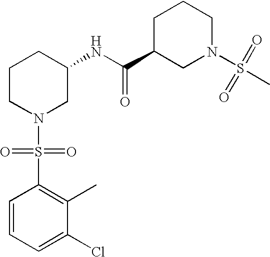

### (3S)-N-(3S)-1-[(3-Chloro-2-methylphenyl)sulfonyl]piperidin-3-yl-1-(methylsulfonyl)piperidine-3-carboxamide

tert-Butyl (3S)-3-[((3S)-1-[(3-chloro-2-methylphenyl)sulfonyl]piperidin-3-ylamino)carbonyl]-piperidine-1-carboxylate (10.0 mg, 200 μmol, prepared as example 73) was treated with 4.0 M of hydrogen chloride in 1,4-dioxane (0.5 mL) at rt for 1 h. The solvent was evaporated in-vacuo and the residue was dissolved in acetonitrile (0.8 mL) and treated with diisopropylethylamine (20.0 μL) and methylsulfonyl chloride (5.0 μL). The resulting mixture was stirred at rt for 30 min. The crude reaction mixture was diluted with MeOH (1.3 mL) and was adjusted to a pH of 2 using TFA and was purified by prep-HPLC to give the desired product. LCMS: (M+H)⁺=478.0/480.0.

### Example 75

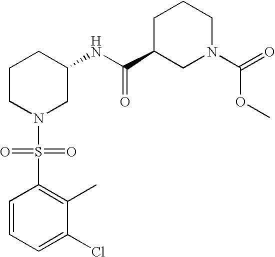

### Methyl (3S)-3-[((3S)-1-[(3-chloro-2-methylphenyl)sulfonyl]piperidin-3-ylamino)carbonyl]piperidine-1-carboxylate

This compound was prepared using procedures analogous to those for example 74. LCMS: (M+H)⁺=458.1/460.1.

### Example 76

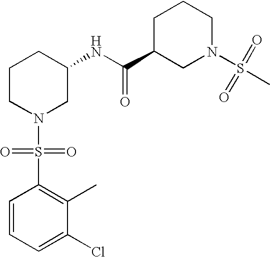

### (3S)-N-(3S)-1-[(3-Chloro-2-methylphenyl)sulfonyl]piperidin-3-yl-1-(methylsulfonyl)piperidine-3-carboxamide

This compound was prepared using procedures analogous to those for example 74. LCMS: (M+H)⁺=478.0/480.0.

### Example 77

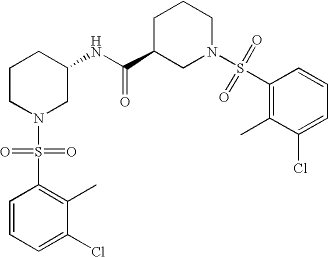

### (3S)-1-[(3-Chloro-2-methylphenyl)sulfonyl]-N-(3S)-1-[(3-chloro-2-methylphenyl)sulfonyl]piperidin-3-ylpiperidine-3-carboxamide

This compound was prepared using procedures analogous to those for example 74. LCMS: (M+H)⁺=588.1/590.1.

### Example 78

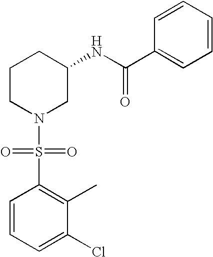

### N-{(3S)-1-[(3-Chloro-2-methylphenyl)sulfonyl]piperidin-3-yl}benzamide

This compound was prepared using procedures analogous to those for example 56. LCMS: (M+H)⁺=393.1/395.0.

### Example 79

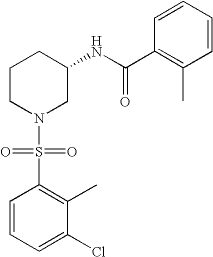

### N-{(3S)-1-[(3-Chloro-2-methylphenyl)sulfonyl]piperidin-3-yl}-2-methylbenzamide

This compound was prepared using procedures analogous to those for example 56. LCMS: (M+H)⁺=407.1/409.1.

### Example 80

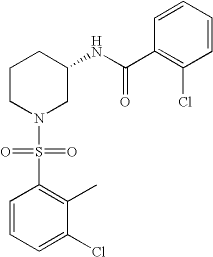

### N-{(3S)-1-[(3-Chloro-2-methylphenyl)sulfonyl]piperidin-3-yl}-2-chlorobenzamide

This compound was prepared using procedures analogous to those for example 56. LCMS: (M+H)⁺=427.0/429.0.

### Example 81

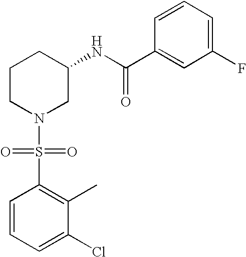

### N-{(3S)-1-[(3-Chloro-2-methylphenyl)sulfonyl]piperidin-3-yl}-3-fluorobenzamide

This compound was prepared using procedures analogous to those for example 56. LCMS: (M+H)⁺=411.0/413.0.

### Example 82

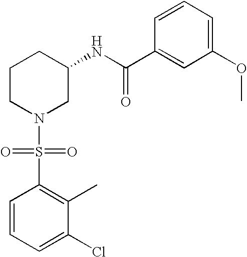

### N-{(3S)-1-[(3-Chloro-2-methylphenyl)sulfonyl]piperidin-3-yl}-3-methoxybenzamide

This compound was prepared using procedures analogous to those for example 56. LCMS: (M+H)⁺=423.1/425.1.

### Example 83

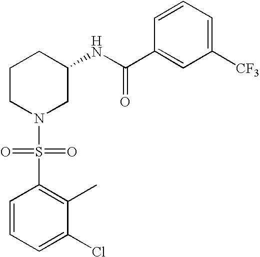

### N-{(3S)-1-[(3-Chloro-2-methylphenyl)sulfonyl]piperidin-3-yl}-3-(trifluoromethyl)benzamide

This compound was prepared using procedures analogous to those for example 56. LCMS: (M+H)⁺=461.0/463.1.

### Example 84

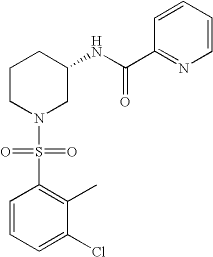

### N-{(3S)-1-[(3-Chloro-2-methylphenyl)sulfonyl]piperidin-3-yl}pyridine-2-carboxamide

This compound was prepared using procedures analogous to those for example 56. LCMS: (M+H)⁺=394.0/396.0.

### Example 85

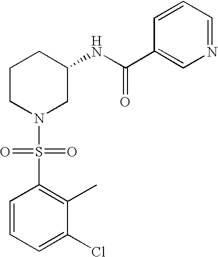

### N-{(3S)-1-[(3-Chloro-2-methylphenyl)sulfonyl]piperidin-3-yl}pyridine-3-carboxamide

This compound was prepared using procedures analogous to those for example 56. LCMS: (M+H)⁺=394.0/396.0.

### Example 86

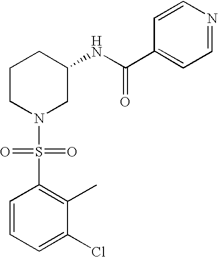

### N-{(3S)-1-[(3-Chloro-2-methylphenyl)sulfonyl]piperidin-3-yl}pyridine-4-carboxamide

This compound was prepared using procedures analogous to those for example 56. LCMS: (M+H)⁺=394.0/396.0.

### Example 87

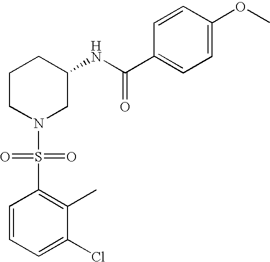

### N-{(3S)-1-[(3-Chloro-2-methylphenyl)sulfonyl]piperidin-3-yl}-4-methoxybenzamide

This compound was prepared using procedures analogous to those for example 56. LCMS: (M+H)⁺=423.1/425.1.

### Example 88

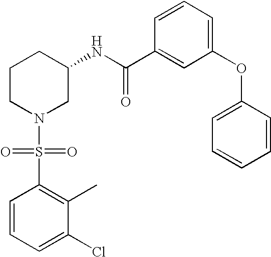

### N-{(3S)-1-[(3-Chloro-2-methylphenyl)sulfonyl]piperidin-3-yl}-3-phenoxybenzamide

This compound was prepared using procedures analogous to those for example 56. LCMS: (M+H)⁺=485.1/487.1.

### Example 89

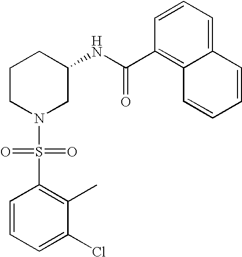

### N-{(3S)-1-[(3-Chloro-2-methylphenyl)sulfonyl]piperidin-3-yl}-1-naphthamide

This compound was prepared using procedures analogous to those for example 56. LCMS: (M+H)⁺=443.1/445.0.

### Example 90

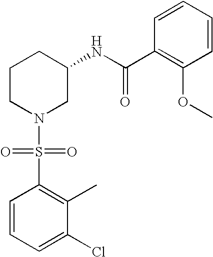

### N-{(3S)-1-[(3-Chloro-2-methylphenyl)sulfonyl]piperidin-3-yl}-2-methoxybenzamide

This compound was prepared using procedures analogous to those for example 56. LCMS: (M+H)⁺=423.1/425.0.

### Example 91

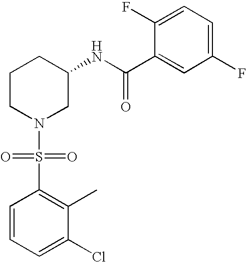

### N-{(3S)-1-[(3-Chloro-2-methylphenyl)sulfonyl]piperidin-3-yl}-2,5-difluorobenzamide

This compound was prepared using procedures analogous to those for example 56. LCMS: (M+H)⁺=429.0/431.0.

### Example 92

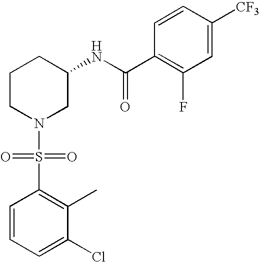

### N-{(3S)-1-[(3-Chloro-2-methylphenyl)sulfonyl]piperidin-3-yl}-2-fluoro-4-(trifluoromethyl)benzamide

This compound was prepared using procedures analogous to those for example 56. LCMS: (M+H)⁺=479.0/481.0.

### Example 93

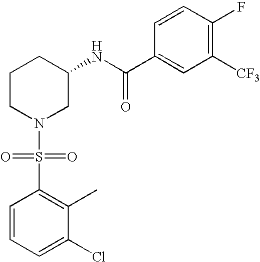

### N-{(3S)-1-[(3-Chloro-2-methylphenyl)sulfonyl]piperidin-3-yl}-4-fluoro-3-(trifluoromethyl)benzamide

This compound was prepared using procedures analogous to those for example 56. LCMS: (M+H)⁺=479.0/481.0.

### Example 94

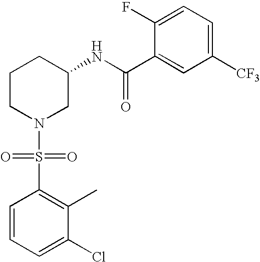

### N-{(3S)-1-[(3-Chloro-2-methylphenyl)sulfonyl]piperidin-3-yl}-2-fluoro-5-(trifluoromethyl)benzamide

This compound was prepared using procedures analogous to those for example 56. LCMS: (M+H)⁺=479.0/481.0.

### Example 95

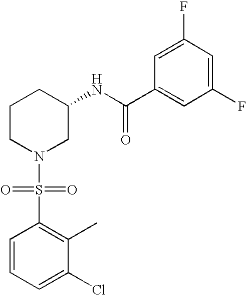

### N-{(3S)-1-[(3-Chloro-2-methylphenyl)sulfonyl]piperidin-3-yl}-3,5-difluorobenzamide

This compound was prepared using procedures analogous to those for example 56. LCMS: (M+H)⁺=429.0/431.0.

### Example 96

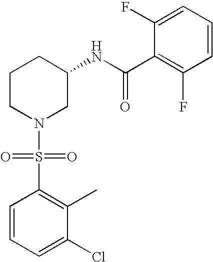

### N-{(3S)-1-[(3-Chloro-2-methylphenyl)sulfonyl]piperidin-3-yl}-2,6-difluorobenzamide

This compound was prepared using procedures analogous to those for example 56. LCMS: (M+H)⁺=429.0/431.0.

### Example 97

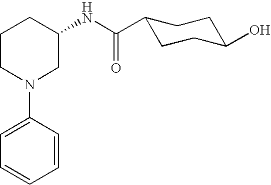

### 4-Hydroxy-N-[(3S)-1-phenylpiperidin-3-yl]cyclohexanecarboxamide

Step 1: tert-Butyl [(3S)-1-phenylpiperidin-3-yl]carbamate

A mixture of tert-butyl (3S)-piperidin-3-ylcarbamate (0.200 g, 0.00100 mol), bromobenzene (211 μL, 0.00200 mol) and sodium tert-butoxide (192 mg, 0.00200 mol) in dimethyl sulfoxide (4.0 mL, 0.056 mol) was irradiated with microwaves to heat the solution to 200° C. for 5 min. The reaction mixture was diluted with water (10 mL) and the solution was extracted with methylene chloride (5×5 mL). The combined organic phases were dried over Na₂SO₄, filtered and concentrated to give the desired product which was used directly in the next step without further purification. LCMS: (M+H)⁺=177.2.

Step 2: (3S)-1-Phenylpiperidin-3-amine dihydrochloride

tert-Butyl [(3S)-1-phenylpiperidin-3-yl]carbamate (48 mg, 0.00017 mol) was dissolved in 2 mL of 4.0 M HCl in dioxane and the resulting solution was stirred at room temperature overnight. The volatiles were removed in-vacuo to afford the desired product as a residue that was used in the next step without further purification.

Step 3: 4-Hydroxy-N-[(3S)-1-phenylpiperidin-3-yl]cyclohexanecarboxamide

4-Methylmorpholine (23 μL 0.00021 mol) was added to a mixture of (3S)-1-phenylpiperidin-3-amine dihydrochloride (0.042 mmol, 0.000042 mol), 4-hydroxycyclohexanecarboxylic acid (6.7 mg, 0.000046 mol) and benzotriazol-1-yloxytris(dimethylamino)phosphonium hexafluorophosphate (0.020 g, 0.000046 mol) in N,N-dimethylformamide (0.5 mL, 0.006 mol). The reaction mixture was stirred at room temperature overnight. The reaction mixture was diluted with methanol (0.8 mL) and adjusted with TFA to pH=2.0. The crude product was purified by prep-LCMS. LCMS: (M+H)⁺=303.2.

### Example 98

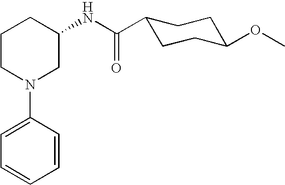

### 4-Methoxy-N-[(3S)-1-phenylpiperidin-3-yl]cyclohexanecarboxamide

This compound was prepared using procedures analogous to those for example 97. LCMS: (M+H)⁺=317.3.

### Example 99

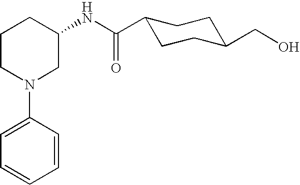

### 4-(Hydroxymethyl)-N-[(3S)-1-phenylpiperidin-3-yl]cyclohexanecarboxamide

This compound was prepared using procedures analogous to those for example 97. LCMS: (M+H)⁺=317.3.

### Example 100

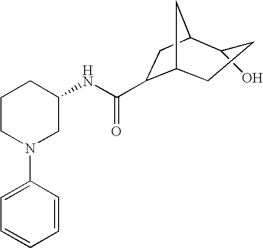

### 2-Hydroxy-N-[(3S)-1-phenylpiperidin-3-yl]bicyclo[3.2.1]octane-6-carboxamide

This compound was prepared using procedures analogous to those for example 97. LCMS: (M+H)⁺=329.3.

### Example 101

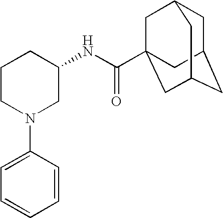

### N-[(3S)-1-Phenylpiperidin-3-yl]adamantane-1-carboxamide

This compound was prepared using procedures analogous to those for example 97. LCMS: (M+H)⁺=339.3.

### Example 102

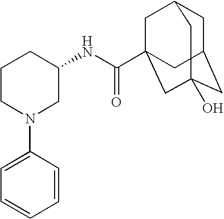

### 3-Hydroxy-N-[(3S)-1-phenylpiperidin-3-yl]adamantane-1-carboxamide

This compound was prepared using procedures analogous to those for example 97. LCMS: (M+H)⁺=355.3.

### Example 103

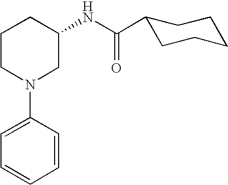

### N-[(3S)-1-Phenylpiperidin-3-yl]cyclohexanecarboxamide

This compound was prepared using procedures analogous to those for example 97. LCMS: (M+H)⁺=287.3.

### Example 104

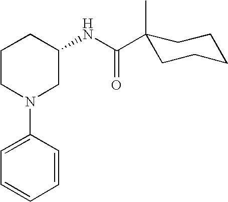

### 1-Methyl-N-[(3S)-1-phenylpiperidin-3-yl]cyclohexanecarboxamide

This compound was prepared using procedures analogous to those for example 97. LCMS: (M+H)⁺=301.3.

### Example 105

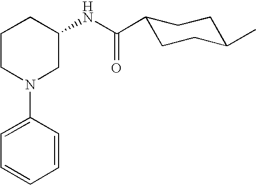

### 4-Methyl-N-[(3S)-1-phenylpiperidin-3-yl]cyclohexanecarboxamide

This compound was prepared using procedures analogous to those for example 97. LCMS: (M+H)⁺=301.3.

### Example 106

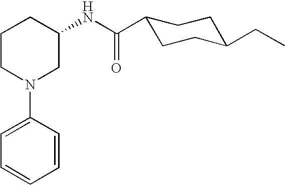

### 4-Ethyl-N-[(3S)-1-phenylpiperidin-3-yl]cyclohexanecarboxamide

This compound was prepared using procedures analogous to those for example 97. LCMS: (M+H)⁺=315.3.

### Example 107

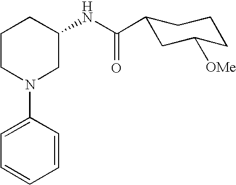

### 3-Methoxy-N-[(3S)-1-phenylpiperidin-3-yl]cyclohexanecarboxamide

This compound was prepared using procedures analogous to those for example 97. LCMS: (M+H)⁺=317.3.

### Example 108

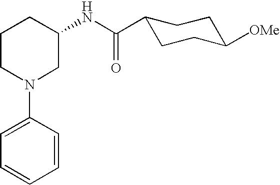

### 4-Methoxy-N-[(3S)-1-phenylpiperidin-3-yl]cyclohexanecarboxamide

This compound was prepared using procedures analogous to those for example 97. LCMS: (M+H)⁺=317.3.

### Example 109

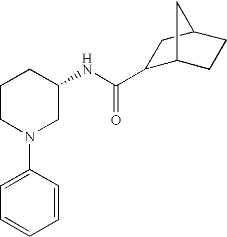

### N-[(3S)-1-Phenylpiperidin-3-yl]bicyclo[2.2.1]heptane-2-carboxamide

This compound was prepared using procedures analogous to those for example 97. LCMS: (M+H)⁺=299.3.

### Example 110

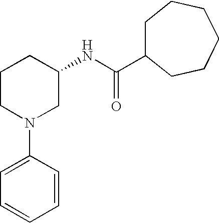

N-[(3S)-1-Phenylpiperidin-3-yl]cycloheptanecarboxamide

This compound was prepared using procedures analogous to those for example 97. LCMS: (M+H)⁺=301.3.

### Example 111

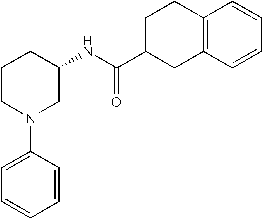

### N-[(3S)-1-Phenylpiperidin-3-yl]-1,2,3,4-tetrahydronaphthalene-2-carboxamide

This compound was prepared using procedures analogous to those for example 97. LCMS: (M+H)⁺=335.2.

### Example 112

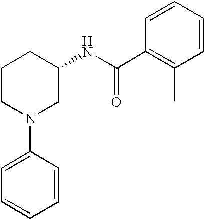

### 2-Methyl-N-[(3S)-1-phenylpiperidin-3-yl]benzamide

This compound was prepared using procedures analogous to those for example 97. LCMS: (M+H)⁺=295.2.

### Example 113

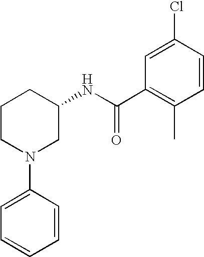

### 5-Chloro-2-methyl-N-[(3S)-1-phenylpiperidin-3-yl]benzamide

This compound was prepared using procedures analogous to those for example 97. LCMS: (M+H)⁺=329.2/331.2.

### Example 114

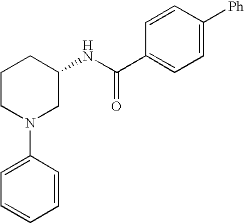

### N-[(3S)-1-Phenylpiperidin-3-yl]biphenyl-4-carboxamide

This compound was prepared using procedures analogous to those for example 97. LCMS: (M+H)⁺=357.2.

### Example 115

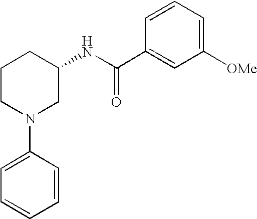

### 3-Methoxy-N-[(3S)-1-phenylpiperidin-3-yl]benzamide

This compound was prepared using procedures analogous to those for example 97. LCMS: (M+H)⁺=311.2.

### Example 116

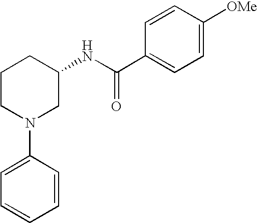

### 4-Methoxy-N-[(3S)-1-phenylpiperidin-3-yl]benzamide

This compound was prepared using procedures analogous to those for example 97. LCMS: (M+H)⁺=311.2.

### Example 117

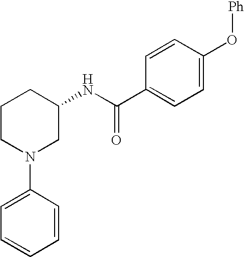

### 4-Phenoxy-N-[(3S)-1-phenylpiperidin-3-yl]benzamide

This compound was prepared using procedures analogous to those for example 97. LCMS: (M+H)⁺=373.2.

### Example 118

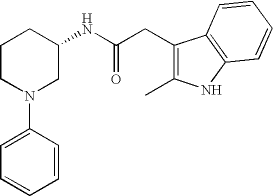

### 2-(2-Methyl-1H-indol-3-yl)-N-[(3S)-1-phenylpiperidin-3-yl]acetamide

This compound was prepared using procedures analogous to those for example 97. LCMS: (M+H)⁺=348.2.

### Example 119

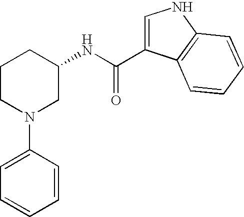

### N-[(3S)-1-Phenylpiperidin-3-yl]-1H-indole-3-carboxamide

This compound was prepared using procedures analogous to those for example 97. LCMS: (M+H)⁺=320.2.

### Example 120

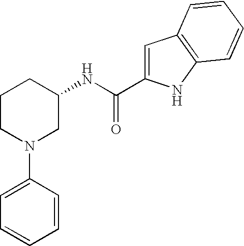

### N-[(3S)-1-Phenylpiperidin-3-yl]-1H-indole-2-carboxamide

This compound was prepared using procedures analogous to those for example 97. LCMS: (M+H)⁺=320.2.

### Example 121

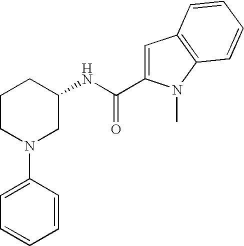

### 1-Methyl-N-[(3S)-1-phenylpiperidin-3-yl]-1H-indole-2-carboxamide

This compound was prepared using procedures analogous to those for example 97. LCMS: (M+H)⁺=334.2.

### Example 122

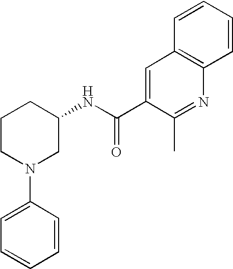

### 2-Methyl-N-[(3S)-1-phenylpiperidin-3-yl]quinoline-3-carboxamide

This compound was prepared using procedures analogous to those for example 97. LCMS: (M+H)⁺=346.2.

### Example 123

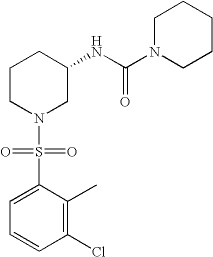

### N-{(3S)-1-[(3-Chloro-2-methylphenyl)sulfonyl]piperidin-3-yl}piperidine-1-carboxamide

Step 1: tert-Butyl {(3S)-1-[(3-chloro-2-methylphenyl)sulfonyl]piperidin-3-yl}carbamate

A solution of tert-butyl (3S)-piperidin-3-ylcarbamate (499 mg, 0.00249 mol; CNH Technologies) and triethylamine (0.52 mL, 0.0037 mol) dissolved in methylene chloride (5.0 mL, 0.078 mol) was cooled to 0° C. and to this was added 3-chloro-2-methylbenzenesulfonyl chloride (0.62 g, 0.0027 mol) (6:56). After stirring for 10 min. the reaction mixture was allowed to gradually warm to rt while stirring for 24 h. The reaction was quenched with water (1:09), diluted with EtOAc and 0.1N HCl and brine were added. The layers were separated and the organic layer was washed with saturated sodium bicarbonate, brine, dried (Na₂SO₄), filtered, and concentrated in-vacuo to afford 1.03 g of the desired product as a white solid. The ¹HNMR confirmed that the desired product was isolated.

Step 2: (3S)-1-[(3-Chloro-2-methylphenyl)sulfonyl]piperidin-3-amine

Trifluoroacetic acid (1.0 mL, 0.013 mol) was added to a solution of tert-butyl {(3S)-1-[(3-chloro-2-methylphenyl)sulfonyl]piperidin-3-yl}carbamate (1.03 g, 0.00265 mol) disssolved in methylene chloride (3.0 mL, 0.047 mol). After stirring for 2 h, the volatiles were removed in-vacuo and the residue was dissolved in methylene chloride and washed with 1 N NaOH, dried (Na₂SO₄), and concentrated in-vacuo to afford 828 mg of the desired product as a white solid. The ¹H NMR confirmed the isolation of the desired product.

Step 3: 4-Nitrophenyl {(3S)-1-[(3-chloro-2-methylphenyl)sulfonyl]piperidin-3-yl}carbamate

(3S)-1-[(3-Chloro-2-methylphenyl)sulfonyl]piperidin-3-amine (404 mg, 0.00140 mol) was dissolved in methylene chloride (1.0E1 mL, 0.16 mol) and to this was added triethylamine (0.39 mL, 0.0028 mol) and p-nitrophenyl chloroformate (342 mg, 0.00170 mol). After stirring at rt for 4 h, the reaction mixture was washed with 0.1 N HCl (2×2 mL) and the combined aq. layer was washed with DCM. The combined organic phases were dried (MgSO₄), filtered, and the volatiles were removed in-vacuo to afford 691 mg of the desired product as a yellow solid. The ¹H NMR confirmed the isolation of the desired product. LCMS: M+H=454.1/456.1. The product was used in the next step without further purification.

Step 4: N-{(3S)-1-[(3-Chloro-2-methylphenyl)sulfonyl]piperidin-3-yl}piperidine-1-carboxamide

Piperidine (11 μL, 0.00011 mol) was added to a solution of 4-nitrophenyl {(3S)-1-[(3-chloro-2-methylphenyl)sulfonyl]piperidin-3-yl}carbamate (25 mg, 0.000055 mol) dissolved in tetrahydrofuran (0.5 mL, 0.006 mol). After 18 h, the volatiles were removed in the residue was dissolved in MeCN/H₂O and purified by prep.-HPLC to afford 19 mg of the desired product as a white powder. ¹H NMR confirmed the isolation of the desired product. LCMS: M+H=400.2/402.2.

### Example 124

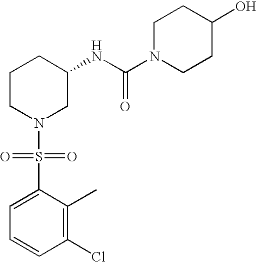

### N-{(3S)-1-[(3-Chloro-2-methylphenyl)sulfonyl]piperidin-3-yl}-4-hydroxypiperidine-1-carboxamide

This compound was prepared using procedures analogous to those for example 123. LCMS: (M+H)⁺=416.2/418.1.

### Example 125

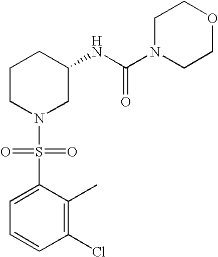

### N-{(3S)-1-[(3-Chloro-2-methylphenyl)sulfonyl]piperidin-3-yl}morpholine-4-carboxamide

This compound was prepared using procedures analogous to those for example 123. LCMS: (M+H)⁺=402.1/404.1.

### Example 126

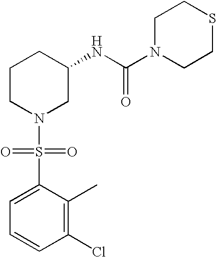

### N-{(3S)-1-[(3-Chloro-2-methylphenyl)sulfonyl]piperidin-3-yl}thiomorpholine-4-carboxamide

This compound was prepared using procedures analogous to those for example 123. LCMS: (M+H)⁺=418.1/420.1.

### Example 127

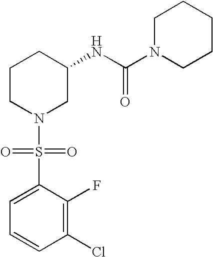

### N-{(3S)-1-[(3-Chloro-2-fluorophenyl)sulfonyl]piperidin-3-yl}piperidine-1-carboxamide

This compound was prepared using procedures analogous to those for example 123. LCMS: (M+H)⁺=404.1/406.1.

### Example 128

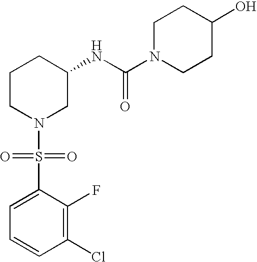

### N-{(3S)-1-[(3-Chloro-2-fluorophenyl)sulfonyl]piperidin-3-yl}-4-hydroxypiperidine-1-carboxamide

This compound was prepared using procedures analogous to those for example 123. LCMS: (M+H)⁺=420.1/422.1.

### Example 129

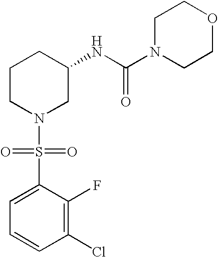

### N-{(3S)-1-[(3-Chloro-2-fluorophenyl)sulfonyl]piperidin-3-yl}morpholine-4-carboxamide

This compound was prepared using procedures analogous to those for example 123. LCMS: (M+H)⁺=406.1/408.1.

### Example 130

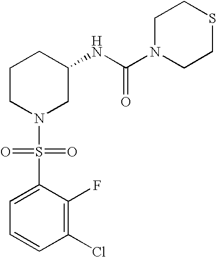

### N-{(3S)-1-[(3-Chloro-2-fluorophenyl)sulfonyl]piperidin-3-yl}thiomorpholine-4-carboxamide

This compound was prepared using procedures analogous to those for example 123. LCMS: (M+H)⁺=422.1/424.1.

### Example 131

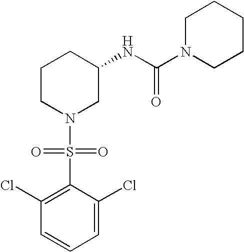

### N-{(3S)-1-[(2,6-Dichlorophenyl)sulfonyl]piperidin-3-yl}piperidine-1-carboxamide

This compound was prepared using procedures analogous to those for example 123. LCMS: (M+H)⁺=420.1/422.1.

### Example 132

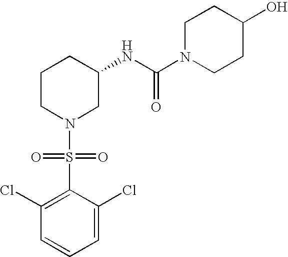

### N-{(3S)-1-[(2,6-Dichlorophenyl)sulfonyl]piperidin-3-yl}-4-hydroxypiperidine-1-carboxamide

This compound was prepared using procedures analogous to those for example 123. LCMS: (M+H)⁺=436.1/438.1.

### Example 133

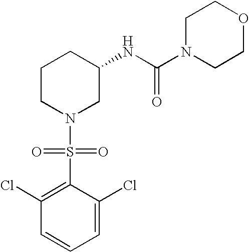

### N-{(3S)-1-[(2,6-Dichlorophenyl)sulfonyl]piperidin-3-yl}morpholine-4-carboxamide

This compound was prepared using procedures analogous to those for example 123. LCMS: (M+H)⁺=422.1/424.1.

### Example 134

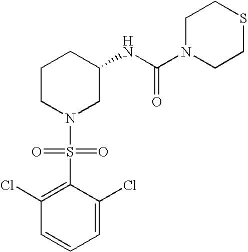

### N-{(3S)-1-[(2,6-Dichlorophenyl)sulfonyl]piperidin-3-yl}thiomorpholine-4-carboxamide

This compound was prepared using procedures analogous to those for example 123. LCMS: (M+H)⁺=438.1/440.0.

### Example 135

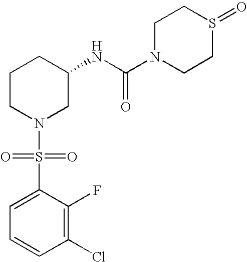

### N-{(3S)-1-[(3-Chloro-2-fluorophenyl)sulfonyl]piperidin-3-yl}thiomorpholine-4-carboxamide 1-oxide

m-Chloroperbenzoic acid (61 mg, 0.00027 mol) was added to a solution of N-{(3S)-1-[(3-chloro-2-fluorophenyl)sulfonyl]piperidin-3-yl}thiomorpholine-4-carboxamide (75 mg, 0.00018 mol) dissolved in methylene chloride (5.0 mL, 0.078 mol) and the solution was stirred at rt for 16 h. The reaction was quenched by the addition of saturated sodium bisulfite and the reaction mixture was allowed to stir for an additional 2 h. The solution was washed thoroughly with 1 N NaOH and the resulting organic layer was washed with brine, dried (Na₂SO₄), filtered, and the volatiles were removed in-vacuo to yield 62 mg of the desired product as a white solid, which was purified by prep-HPLC. LCMS (M+H)⁺=438.1/440.1.

### Example 136

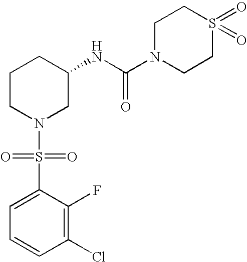

### N-{(3S)-1-[(3-Chloro-2-fluorophenyl)sulfonyl]piperidin-3-yl}thiomorpholine-4-carboxamide 1,1-dioxide

This compound was prepared using procedures analogous to those for example 135. LCMS: (M+H)⁺=454.1/456.1.

### Example 137

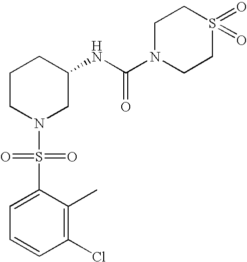

### N-{(3S)-1-[(3-Chloro-2-methylphenyl)sulfonyl]piperidin-3-yl}thiomorpholine-4-carboxamide 1,1-dioxide

This compound was prepared using procedures analogous to those for example 135. LCMS: (M+H)⁺=450.1/452.1.

### Example 138

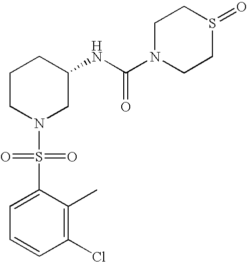

### N-{(3S)-1-[(3-Chloro-2-methylphenyl)sulfonyl]piperidin-3-yl}thiomorpholine-4-carboxamide 1-oxide

This compound was prepared using procedures analogous to those for example 135. LCMS: (M+H)⁺=434.1/436.0.

### Example 139

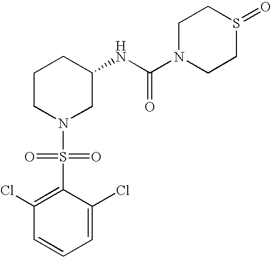

### N-{(3S)-1-[(2,6-Dichlorophenyl)sulfonyl]piperidin-3-yl}thiomorpholine-4-carboxamide 1-oxide

This compound was prepared using procedures analogous to those for example 135. LCMS: (M+H)⁺=454.0/456.1.

### Example 140

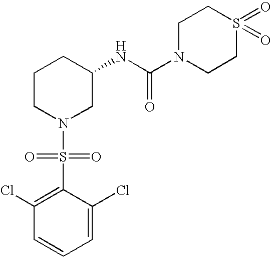

### N-{(3S)-1-[(2,6-Dichlorophenyl)sulfonyl]piperidin-3-yl}thiomorpholine-4-carboxamide 1,1-dioxide

This compound was prepared using procedures analogous to those for example 135. LCMS: (M+H)⁺=470.0/472.0.

### Example 141

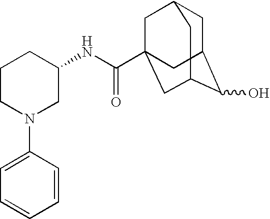

### 4-Hydroxy-N-[(3S)-1-phenylpiperidin-3-yl]adamantane-1-carboxamide

Step 1: tert-Butyl (3S)-3-{[(4-oxo-1-adamantyl)carbonyl]amino}piperidine-1-carboxylate

Oxalyl chloride (233 μL, 0.00275 mol) was added to 4-oxoadamantane-1-carboxylic acid (97.08 mg, 0.0004998 mol) in methylene chloride (10 mL) at rt followed by 2 drops of DMF. After stirring the mixture at rt for 2 h, the volatiles were evaporated under reduced pressure. The residue was azeotropically evaporated twice with toluene and the resulting residue was dissolved in DCM (10 mL). To the solution was added tert-butyl (3S)-3-aminopiperidine-1-carboxylate (100.1 mg, 0.0004998 mol) and N,N-diisopropylethylamine (0.18 mL, 0.0010 mol). After stirring at rt for 1 h, the reaction mixture was diluted with DCM (100 mL) and washed with water, 1N HCl, and brine. The organic phase was dried over Na₂SO₄, filtered, and concentrated in-vacuo to provide the desired product. LCMS: (M-t-Bu+1)⁺=321.2.

Step 2: tert-butyl (3S)-3-{[(4-hydroxy-1-adamantyl)carbonyl]amino}piperidine-1-carboxylate

1.0 M of L-selectride® in tetrahydrofuran (0.50 mL) was added to a solution of tert-butyl (3S)-3-{[(4-oxo-1-adamantyl)carbonyl]amino}piperidine-1-carboxylate (75 mg, 0.00020 mol) in tetrahydrofuran (1.0 mL, 0.012 mol) at −78° C. The mixture was stirred at −78° C. for 30 min. and was then quenched with ice-water. The mixture was extracted with ethyl acetate (3×2 mL). The combined organic phases were washed with brine (2 mL), dried over Na₂SO₄, filtered, and concentrated under reduced pressure. The residue was purified by Combiflash, eluting with ethyl acetate/hexanes, to provide the desired product. LCMS: (M-t-Bu+H)⁺=323.2.

Step 3: 4-Hydroxy-N-[(3S)-piperidin-3-yl]adamantane-1-carboxamide hydrochloride

tert-Butyl (3S)-3-{[(4-hydroxy-1-adamantyl)carbonyl]amino}piperidine-1-carboxylate (75 mg, 0.00020 mol) was treated with 4.0 M of hydrogen chloride in 1,4-dioxane (0.30 mL) at rt for 30 min. The volatiles were evaporated and the residue was dried under reduced pressure to afford the desired product. LCMS: (M+H)⁺=315.4.

Step 4: 4-Hydroxy-N-[(3S)-1-phenylpiperidin-3-yl]adamantane-1-carboxamide

A mixture of 4-hydroxy-N-[(3S)-piperidin-3-yl]adamantane-1-carboxamide hydrochloride (15.7 mg, 0.0000500 mol), bromobenzene (10.5 μL, 0.000100 mol) and sodium tert-butoxide (9.61 mg, 0.000100 mol) in dimethyl sulfoxide (0.50 mL, 0.0070 mol) was irradiated with microwaves at 200° C. for 5 min. The mixture was diluted with methanol (1.3 mL) and adjusted with TFA to pH=2.0. The resulting solution was purified by prep.-HPLC to give the equatorial and axial hydroxyl products. LCMS: (M+H)⁺=355.2.

### Example 142

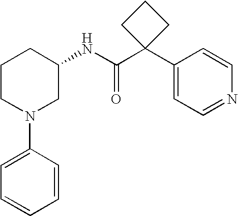

### N-[(3S)-1-Phenylpiperidin-3-yl]-1-pyridin-4-ylcyclobutanecarboxamide

This compound was prepared using procedures analogous to those described for the synthesis of example 97, steps 1-3. LCMS: (M+H)⁺=336.0.

### Example 143

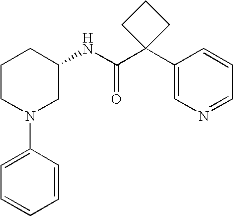

### N-[(3S)-1-Phenylpiperidin-3-yl]-1-pyridin-3-ylcyclobutanecarboxamide

This compound was prepared using procedures analogous to those described for the synthesis of example 97, steps 1-3. LCMS: (M+H)⁺=336.0.

### Example 144

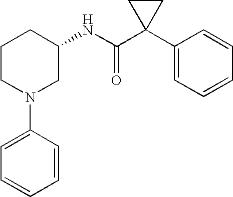

### 1-Phenyl-N-[(3S)-1-phenylpiperidin-3-yl]cyclopropanecarboxamide

This compound was prepared using procedures analogous to those described for the synthesis of example 97, steps 1-3. LCMS: (M+H)⁺=321.1.

### Example 145

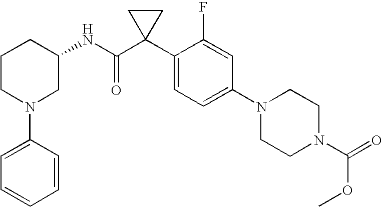

### Methyl 4-{3-fluoro-4-[1-({[(3S)-1-phenylpiperidin-3-yl]amino}carbonyl)cyclopropyl]phenyl}piperazine-1-carboxylate

Step 1. 1-(4-Bromo-2-fluorophenyl)cyclopropanecarboxylic acid

To a stirred mixture of the (4-bromo-2-fluorophenyl)acetonitrile (12.53 g, 0.05854 mol), benzyltriethylammonium chloride (0.9 g, 0.004 mol), and 1-bromo-2-chloro-ethane (9.70 mL, 0.117 mol) was added dropwise sodium hydroxide, 50% aqueous solution (21.00 mL, 0.5484 mol) at 50° C. After stirring for 16 h, the reaction mixture was diluted with water, 1,2-ethanediol (65.00 mL, 1.166 mol), and sodium hydroxide, 50% aqueous solution (5 mL). The resulting mixture was heated at 100° C. for 16 h. The reaction mixture was extracted with diethyl ether and the aqueous layer was acidified to pH˜2 and the product precipitated out and was collected by filtration and used in the subsequent reaction without further purification.

Step 2. 1-{4-[4-(tert-Butoxycarbonylpiperazin-1-yl]-2-fluorophenyl}cyclopropane carboxylic acid

A mixture of 1-(4-bromo-2-fluorophenyl)cyclopropanecarboxylic acid (2.390 g, 0.009225 mol), tert-butyl piperazine-1-carboxylate (2.126 g, 0.01107 mol), sodium tert-butoxide (2.194 g, 0.02214 mol), palladium acetate (62 mg, 0.00028 mol) and 2-(di-t-butylphosphino)biphenyl (165 mg, 0.000554 mol) in anhydrous 1,4-dioxane (30.0 mL, 0.384 mol) was refluxed (oil bath temperature 110° C.) overnight. The reaction mixture was poured into cold saturated NH₄Cl (60 mL), acidified to pH=6 with 1 N HCl, and extracted with ethyl acetate (2×). The combined organic layers were washed with brine, dried over MgSO₄, filtered and concentrated in-vacuo. The residue was purified by CombiFlash eluting with 0-10% methanol in methylene chloride to give the product (1.762 g, 52% in yield). LCMS: (M-t-Bu+H)⁺=309.1.

Step 3. tert-Butyl 4-{3-fluoro-4-[1-({[(3S)-1-phenylpiperidin-3-yl]amino}carbony) cyclopropyl]phenyl}piperazine-1-carboxylate

This compound was prepared using a procedure that was analogous to that described for the synthesis of example 97, steps 1-3. LCMS: (M-t-Bu+2H)⁺=467.1

Step 4. Methyl 4-{3-fluoro-4-[1-({[(3S)-1-phenylpiperidin-3-yl]amino}carbonyl) cyclopropyl]phenyl}piperazine-1-carboxylate

This compound was prepared using a procedure that was analogous to that described for the synthesis of example 9, steps 2 and 3. LCMS: (M+H)⁺=481.1

### Example 146

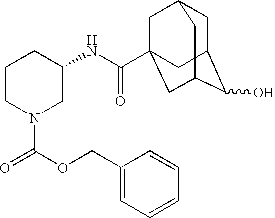

### Benzyl (3S)-3-{[(4-hydroxy-1-adamantyl)carbonyl]amino}piperidine-1-carboxylate

This compound was prepared using a procedure that was analogous to that described for the synthesis of example 9, steps 1-3 using the appropriate carbonyl chloride. LCMS: (M+H)⁺=413.2.

### Example 147

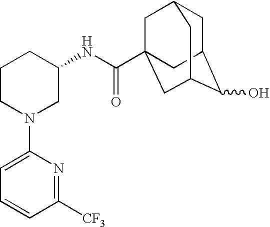

### 4-Hydroxy-N-{(3S)-1-[6-(trifluoromethyl)pyridin-2-yl]piperidin-3-yl}adamantane-1-carboxamide

This compound was prepared using a procedure that was analogous to that described for the synthesis of example 97, steps 1-3. LCMS: (M+H)⁺=424.2.

### Example 148

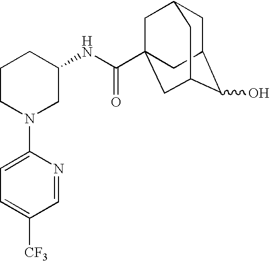

### 4-Hydroxy-N-{(3S)-1-[5-(trifluoromethyl)pyridin-2-yl]piperidin-3-yl}adamantane-1-carboxamide

This compound was prepared using a procedure that was analogous to that described for the synthesis of example 97, steps 1-3. LCMS: (M+H)⁺=424.2.

### Example 149

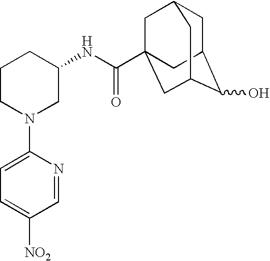

### 4-Hydroxy-N-[(3S)-1-(5-nitropyridin-2-yl)piperidin-3-yl]adamantane-1-carboxamide

This compound was prepared using a procedure that was analogous to that described for the synthesis of example 97, steps 1-3. LCMS: (M+H)⁺=401.2.

### Example 150

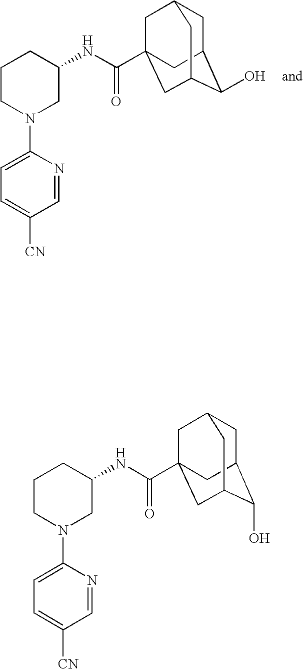

### N-[(3S)-1-(5-Cyanopyridin-2-yl)piperidin-3-yl]-4-hydroxyadamantane-1-carboxamide

This compound was prepared using a procedure that was analogous to that described for the synthesis of example 97, steps 1-3. LCMS: (M+H)⁺=381.1.

### Example 151

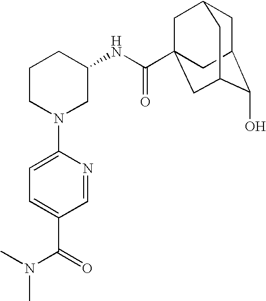

### 6-((3S)-3-{[(4-Hydroxy-1-adamantyl)carbonyl]amino}piperidin-1-yl)-N,N-dimethylnicotinamide

This compound was prepared using a procedure that was analogous to that described for the synthesis of example 97, steps 1-3. LCMS: (M+H)⁺=427.3.

### Example 152

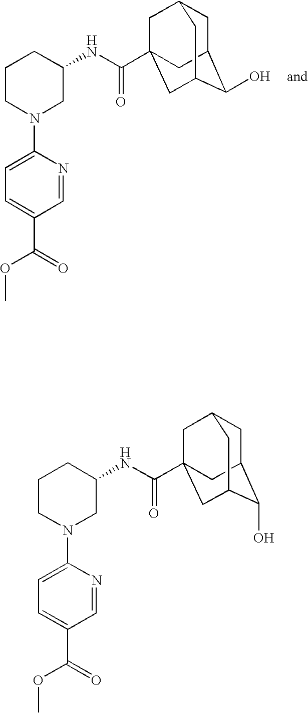

### Methyl 6-((3S)-3-{[(4-hydroxy-1-adamantyl)carbonyl]amino}piperidin-1-yl)nicotinate

This compound was prepared using a procedure that was analogous to that described for the synthesis of example 97, steps 1-3. LCMS: (M+H)⁺=414.2.

### Example 153

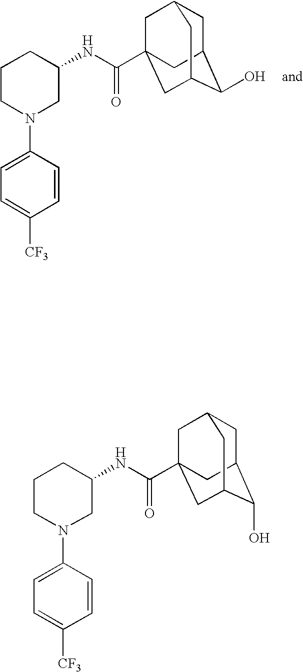

### 4-hydroxy-N-{(3S)-1-[4-(trifluoromethyl)phenyl]piperidin-3-yl}adamantane-1-carboxamide

A mixture of 4-hydroxy-N-[(3S)-piperidin-3-yl]adamantane-1-carboxamide (20.9 mg, 0.0000750 mol), 1-bromo-4-(trifluoromethyl)benzene (25.3 mg, 0.000112 mol), sodium tert-butoxide (10.8 mg, 0.000112 mol), palladium acetate (0.50 mg, 0.0000022 mol) and 2-(di-t-butylphosphino)biphenyl (1.3 mg, 0.0000045 mol) was vacuumed and charged with nitrogen. To the mixture was added 1,4-dioxane (0.75 mL, 0.0096 mol) and the resulting mixture was refluxed for 16 h. After cooling to ambient temperature, the reaction mixture was filtered and the filtrate was adjusted with TFA to pH=2.0 and was purified by prep.-HPLC to give the desired product. LCMS: (M+H)⁺=423.2.

### Example 154

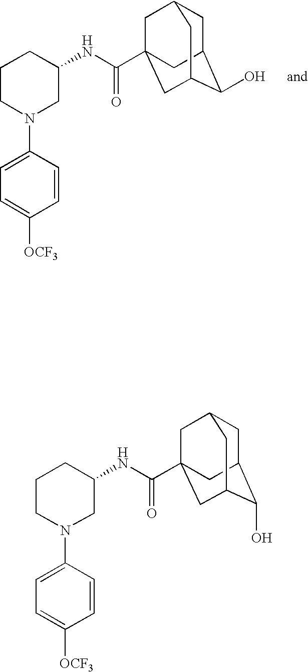

### 4-Hydroxy-N-{(3S)-1-[4-(trifluoromethoxy)phenyl]piperidin-3-yl}adamantane-1-carboxamide

This compound was prepared using a procedure that was analogous to that described for the synthesis of example 153. LCMS: (M+H)⁺=439.2.

### Example 155

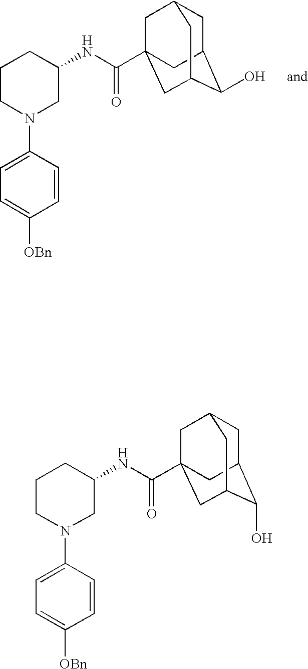

### N-{(3S)-1-[4-(Benzyloxy)phenyl]piperidin-3-yl}-4-hydroxyadamantane-1-carboxamide

This compound was prepared using a procedure that was analogous to that described for the synthesis of example 153. LCMS: (M+H)⁺=461.3.

### Example 156

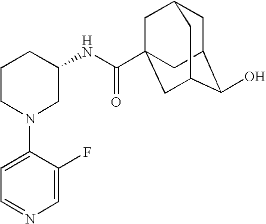

### N-[(3S)-1-(3-Fluoropyridin-4-yl)piperidin-3-yl]-4-hydroxyadamantane-1-carboxamide

This compound was prepared using a procedure that was analogous to that described for the synthesis of example 97, steps 1-3. LCMS: (M+H)⁺=374.2.

### Example 157

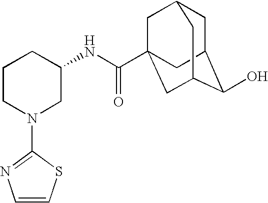

### 4-Hydroxy-N-[(3S)-1-(1,3-thiazol-2-yl)piperidin-3-yl]adamantane-1-carboxamide

This compound was prepared using a procedure that was analogous to that described for the synthesis of example 97, steps 1-3. LCMS: (M+H)⁺=362.2.

### Example 158

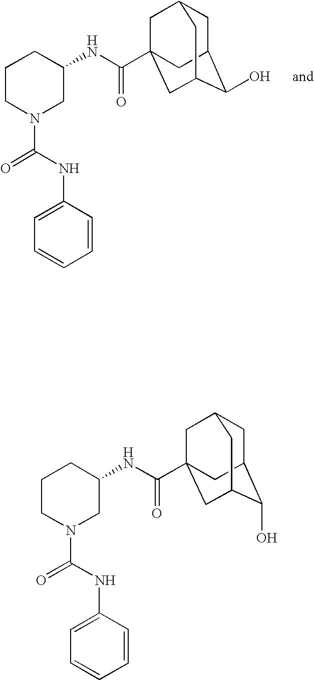

### (3S)-3-{[(4-Hydroxy-1-adamantyl)carbonyl]amino}-N-phenylpiperidine-1-carboxamide

This compound was prepared using a procedure that was analogous to that described for the synthesis of example 9, steps 1-3 using the appropriate carbonyl chloride reagent. LCMS: (M+H)⁺=398.2.

### Example 159

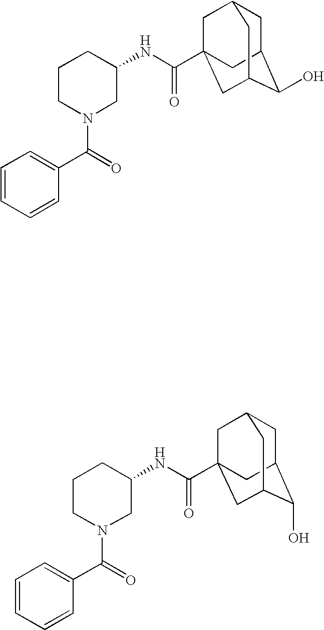

### N-[(3S)-1-Benzoylpiperidin-3-yl]-4-hydroxyadamantane-1-carboxamide

This compound was prepared using a procedure that was analogous to that described for the synthesis of example 9, steps 1-3 using the appropriate carbonyl chloride reagent. LCMS: (M+H)⁺=383.2.

### Example 160

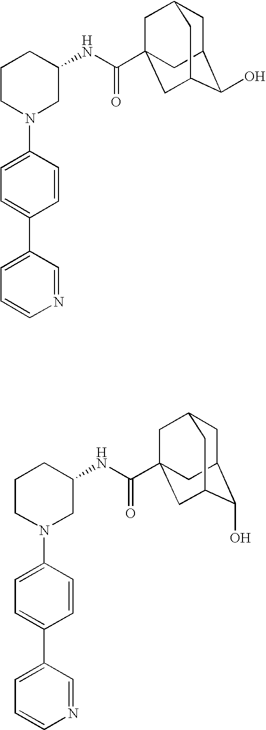

### 4-Hydroxy-N-[(3S)-1-(4-pyridin-3-ylphenyl)piperidin-3-yl]adamantane-1-carboxamide

This compound was prepared using a procedure that was analogous to that described for the synthesis of example 97, steps 1-3. LCMS: (M+H)⁺=432.2.

### Example 161

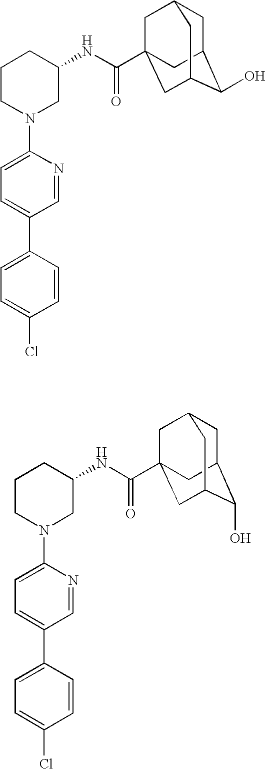

### N-{(3S)-1-[5-(4-Chlorophenyl)pyridin-2-yl]piperidin-3-yl}-4-hydroxyadamantane-1-carboxamide

This compound was prepared using a procedure that was analogous to that described for the synthesis of example 97, steps 1-3. LCMS: (M+H)⁺=466.2/468.2.

### Example 162

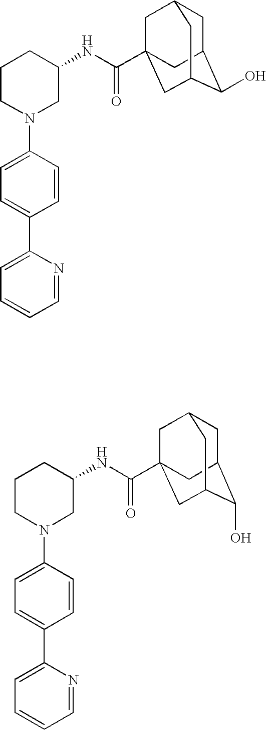

### 4-Hydroxy-N-[(3S)-1-(4-pyridin-2-ylphenyl)piperidin-3-yl]adamantane-1-carboxamide

This compound was prepared using a procedure that was analogous to that described for the synthesis of example 97, steps 1-3. LCMS: (M+H)⁺=432.2.

### Example 163

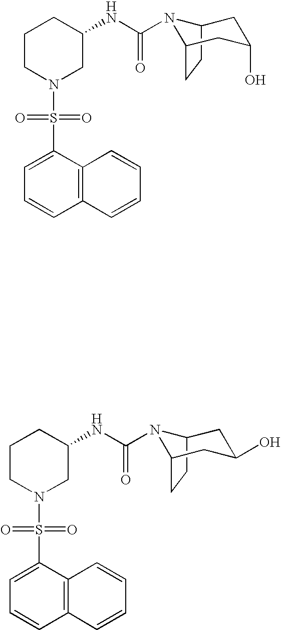

### (1S,5S)-3-Hydroxy-N-[(3S)-1-(1-naphthylsulfonyl)piperidin-3-yl]-8-azabicyclo[3.2.1]octane-8-carboxamide

Step 1. tert-Butyl (3S)-3-{[(4-nitrophenoxy)carbonyl]amino}piperidine-1-carboxylate

This compound was prepared using a procedure that was analogous to that described for the synthesis of example 123, step 3 starting from tert-butyl (3S)-3-aminopiperidine-1-carboxylate. LCMS: (M+Na)⁺=388.1; (M+H-Boc)⁺=266.1.

Step 2. tert-Butyl (3S)-3-({[(1S,5S)-3-hydroxy-8-azabicyclo[3.2.1]oct-8-yl]carbonyl}amino)piperidine-1-carboxylate

This compound was prepared using a procedure that was analogous to that described for the synthesis of example 123, step 4 starting from tert-butyl (3S)-3-{[(4-nitrophenoxy)-carbonyl]amino}-piperidine-1-carboxylate and (1S,5S)-8-azabicyclo[3.2.1]octan-3-ol hydrochloride. LCMS: (M+Na)⁺=376.2.

Step 3. (1S,5S)-3-Hydroxy-N-[(3S)-piperidin-3-yl]-8-azabicyclo[3.2.1]octane-8-carboxamide hydrochloride

This compound was prepared using a procedure that was analogous to that described for the synthesis of example 97, step 2. LCMS: (M+H)⁺=290.3

Step 4. (1S,5S)-3-Hydroxy-N-[(3S)-1-(-naphthylsulfonyl)piperidin-3-yl]-8-azabicyclo[3.2.1]octane-8-carboxamide

This compound was prepared using a procedure that was analogous to that described for the synthesis of example 123, step 1. LCMS: (M+H)⁺=444.2.

### Example 164

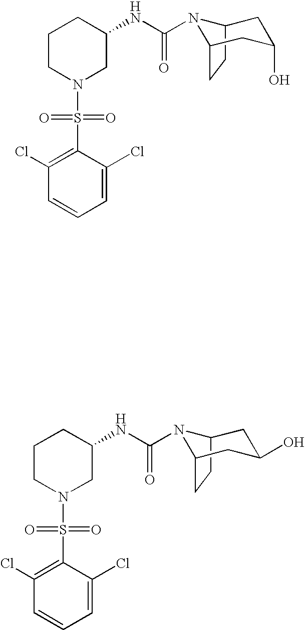

### (1S,5S)-N-{(3S)-1-[(2,6-Dichlorophenyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+1)⁺=462.1/464.1.

### Example 165

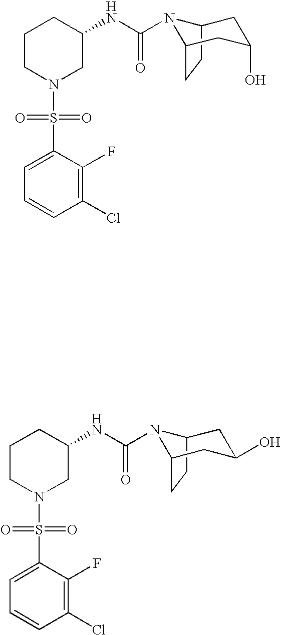

### (1S,5S)-N-{(3S)-1-[(3-Chloro-2-fluorophenyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+H)⁺=446.1/448.1.

### Example 166

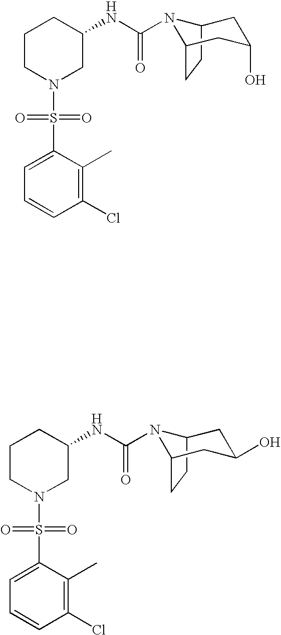

### (1S,5S)-N-{(3S)-1-[(3-chloro-2-methylphenyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+1)⁺=442.1/444.1.

### Example 167

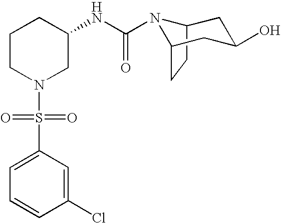

### (1S,5S)-N-{(3S)-1-[(3-chlorophenyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+H)⁺=428.1/430.1.

### Example 168

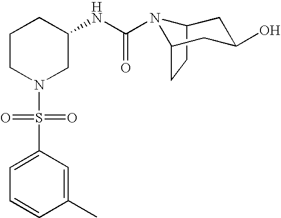

### (1S,5S)-3-Hydroxy-N-{(3S)-1-[(3-methylphenyl)sulfonyl]piperidin-3-yl}-8-azabicyclo[3.2.1]octane-8-carboxamide

This compound was prepared using a procedure that was analogous to that described for the synthesis of example 163, steps 1-4. LCMS: (M+H)⁺=408.2.

### Example 169

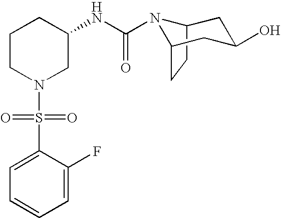

### (1S,5S)-N-{(3S)-1-[(2-Fluorophenyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+H)⁺=412.2.

### Example 170

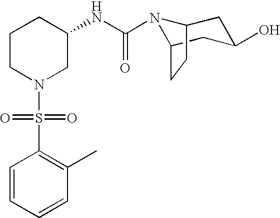

### (1S,5S)-3-Hydroxy-N-{(3S)-1-[(2-methylphenyl)sulfonyl]piperidin-3-yl}-8-azabicyclo[3.2.1]octane-8-carboxamide

This compound was prepared using a procedure that was analogous to that described for the synthesis of example 163, steps 1-4. LCMS: (M+1)⁺=408.1.

### Example 171

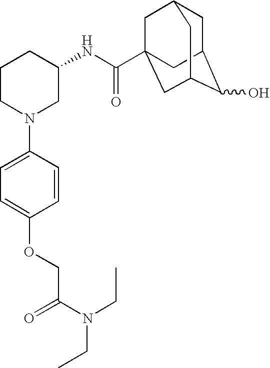

### N-((3S)-1-{4-[2-(Diethylamino)-2-oxoethoxyl]phenyl}piperidin-3-yl)-4-hydroxyadamantane-1-carboxamide

Step 1. Benzyl (3S)-3-{[(4-oxo-1-adamantyl)carbonyl]amino}piperidine-1-carboxylate

Oxalyl chloride (1.50 mL, 0.0177 mol) was added to 4-oxoadamantane-1-carboxylic acid (583 mg, 0.00300 mol) in methylene chloride (10 mL) at rt followed by 2 drops of DMF. The mixture was stirred at rt for 2 h. The volatiles were evaporated under reduced pressure and the residue was azeotropically evaported with toluene twice. The residue was dissolved in DCM (10 mL) and to the solution was added benzyl (3S)-3-aminopiperidine-1-carboxylate hydrochloride (812.6 mg, 0.003001 mol) and N,N-diisopropylethylamine (1.20 mL, 0.00689 mol). The mixture was stirred at rt for 1 h. The reaction mixture was diluted with DCM (100 mL) and washed with water, 1N HCl and brine. The organic phase was dried over Na₂SO₄, filtered and concentrated to give the desired product.

Step 2. Benzyl (3S)-3-{[(4-hydroxy-1-adamantyl)carbonyl]amino}piperidine-1-carboxylate

Sodium borohydride (20.0 mg, 0.000529 mol) was added to a solution of benzyl (3S)-3-{[(4-oxo-1-adamantyl)carbonyl]amino}piperidine-1-carboxylate (102.8 mg, 0.0002504 mol) in methanol (2.0 mL, 0.049 mol) at rt. After stirring at rt for 30 min, the reaction mixture was diluted with ethyl acetate (5 mL), washed with 1N NaOH, water and brine, dried over Na₂SO₄, filtered, and concentrated under reduced pressure. The residue was purified by Combiflash with ethyl acetate/heaxane to give a mixture of two isomers in a ratio of 1:1.

Step 3. 4-Hydroxy-N-[(3S)-piperidin-3-yl]adamantane-1-carboxamide

Benzyl (3S)-3-{[(4-hydroxy-1-adamantyl)carbonyl]amino}piperidine-1-carboxylate (0.900 g, 0.00218 mol) in methanol (15 mL) was hydrogenized with palladium on barium sulfate (25 mg, 0.00023 mol) under an atmosphere of hydrogen using a ballon for 2 h. The mixture was filtered and the filtrate was concentrated. The residue was dried under high vacuum to give the desired product. LCMS: (M+H)⁺=279.1.

Step 4. N-((3S)-1-{4-[2-(Diethylamino)-2-oxoethoxy]phenyl}piperidin-3-yl)-4-hydroxyadamantane-1-carboxamide

A mixture of 4-hydroxy-N-[(3S)-piperidin-3-yl]adamantane-1-carboxamide (18.1 mg, 0.0000650 mol), 2-(4-chlorophenoxy)-N,N-diethylacetamide (23.6 mg, 0.0000975 mol), sodium tert-butoxide (9.37 mg, 0.0000975 mol), palladium acetate (0.44 mg, 0.0000020 mol) and 2-(di-tert-butylphosphino)biphenyl (1.2 mg, 0.0000039 mol) was placed in a 10-mL round-bottomed flask equipped with a stirring bar and reflux condenser and was evacuated and charged with nitrogen. To the mixture was added 1,4-dioxane (0.65 mL, 0.0083 mol) and the resulting mixture was refluxed overnight. After cooling, the mixture was filtered and the filtrate was adjusted with TFA to pH=2.0 and was purified by prep.-HPLC to give the desired product. LCMS: (M+H)⁺=484.2.

### Example 172

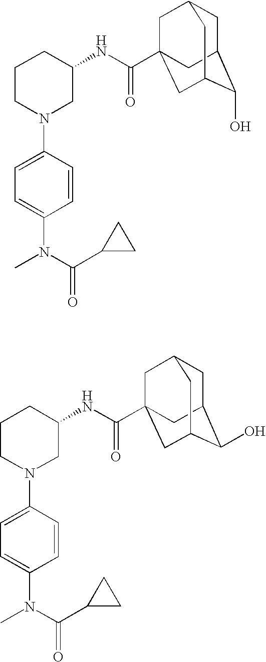

### N-((3S)-1-{4-[(Cyclopropylcarbonyl)(methyl)amino]phenyl}piperidin-3-yl)-4-hydroxyadamantane-1-carboxamide

This compound was prepared using a procedure that was analogous to that described for the synthesis of example 171, steps 1-4. LCMS: (M+H)⁺=452.3.

### Example 173

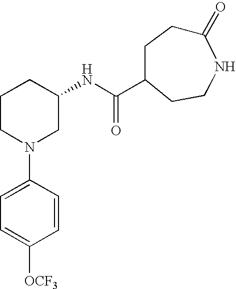

### 7-Oxo-N-{(3S)-1-[4-(trifluoromethoxy)phenyl]piperidin-3-yl}azepane-4-carboxamide

This compound was prepared using a procedure that was analogous to that described for the synthesis of example 171, steps 1-4. The two pure diastereoisomers were separated by prep-HPLC. LCMS: (M+H)⁺=400.1.

### Example 174

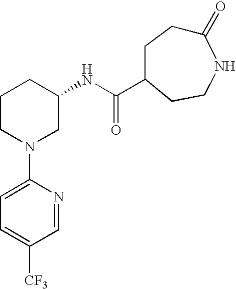

### 7-Oxo-N-{(3S)-1-[5-(trifluoromethyl)pyridin-2-yl]piperidin-3-yl}azepane-4-carboxamide

This compound was prepared using a procedure that was analogous to that described for the synthesis of example 171, steps 1-4. The two pure diastereoisomers were separated by prep-HPLC. LCMS: (M+H)⁺=385.2.

### Example 175

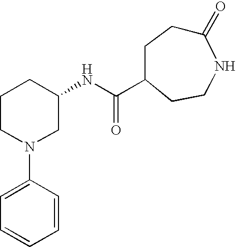

### 7-Oxo-N-[(3S)-1-phenylpiperidin-3-yl]azepane-4-carboxamide

This compound was prepared using a procedure that was analogous to that described for the synthesis of example 171, steps 1-4. The two pure diastereoisomers were separated by prep-HPLC. LCMS: (M+H)⁺=316.2.

### Example 176

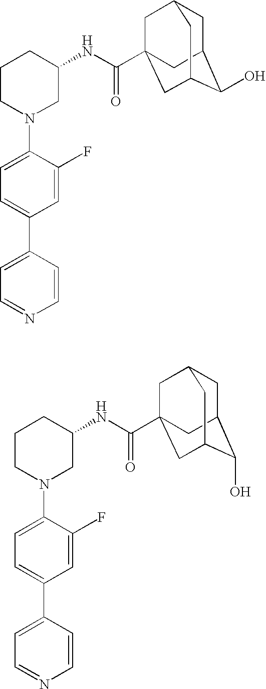

### N-[(3S)-1-(2-Fluoro-4-pyridin-4-ylphenyl)piperidin-3-yl]-4-hydroxyadamantane-1-carboxamide

This compound was prepared using a procedure that was analogous to that described for the synthesis of example 171, steps 1-4. LCMS: (M+H)⁺=450.2.

### Example 177

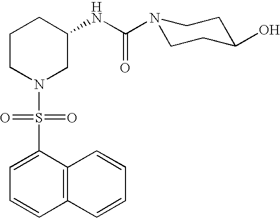

### 4-Hydroxy-N-[(3S)-1-(1-naphthylsulfonyl)piperidin-3-yl]piperidine-1-carboxamide

Step 1. tert-Butyl (3S)-3-{[(4-hydroxypiperidin-1-yl)carbonyl]amino}piperidine-1-carboxylate

This compound was prepared using a procedure that was analogous to that described for the synthesis of example 123, steps 3 & 4. LCMS: (M+H)⁺=328.2; (M+H-Boc)⁺=228.2.

Step 2. 4-Hydroxy-N-[(3S)-1-(1-naphthylsulfonyl)piperidin-3-yl]piperidine-1-carboxamide

This compound was prepared using a procedure that was analogous to that described for the synthesis of example 9, steps 2 & 3. LCMS: (M+H)⁺=418.1.

### Example 178

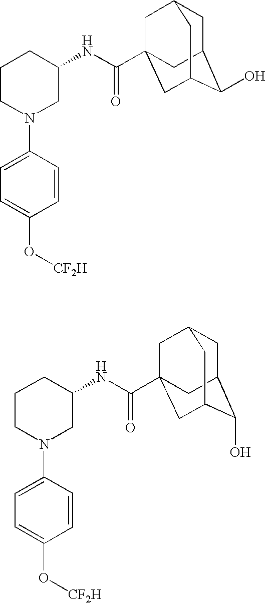

### N-{(3S)-1-[4-(Difluoromethoxy)phenyl]piperidin-3-yl}-4-hydroxyadamantane-1-carboxamide

This compound was prepared using a procedure that was analogous to that described for the synthesis of example 171, steps 1-4. LCMS: (M+H)⁺=421.2.

### Example 179

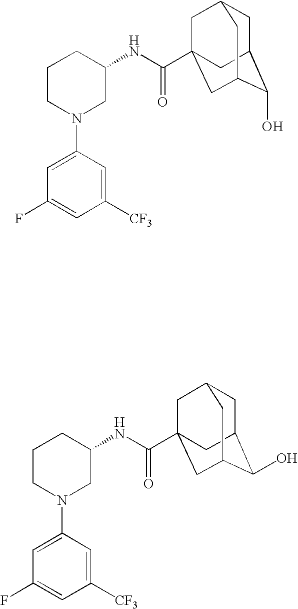

### N-{(3S)-1-[3-Fluoro-5-(trifluoromethyl)phenyl]piperidin-3-yl}-4-hydroxyadamantane-1-carboxamide

This compound was prepared using a procedure that was analogous to that described for the synthesis of example 171, steps 1-4. LCMS: (M+H)⁺=441.2.

### Example 180

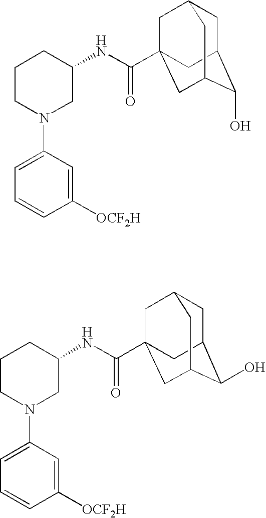

### N-{(3S)-1-[3-(Difluoromethoxy)phenyl]piperidin-3-yl}-4-hydroxyadamantane-1-carboxamide

This compound was prepared using a procedure that was analogous to that described for the synthesis of example 171, steps 1-4. LCMS: (M+H)⁺=421.2.

### Example 181

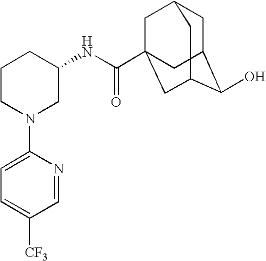

### 4-Hydroxy-N-{(3S)-1-[5-(trifluoromethyl)pyridin-2-yl]piperidin-3-yl}adamantane-1-carboxamide

This compound was prepared using a procedure that was analogous to that described for the synthesis of example 171, steps 1-4. LCMS: (M+H)⁺=424.2.

### Example 182

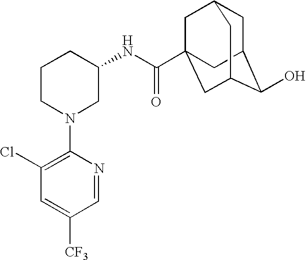

### N-{(3S)-1-[3-Chloro-5-(trifluoromethyl)pyridin-2-yl]piperidin-3-yl}-4-hydroxyadamantane-1-carboxamide

This compound was prepared using a procedure that was analogous to that described for the synthesis of example 171, steps 1-4. LCMS: (M+H)⁺=458.1/460.1.

### Example 183

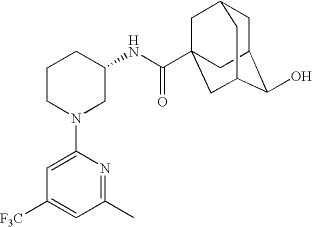

### 4-Hydroxy-N-{(3S)-1-[6-methyl-4-(trifluoromethyl)pyridin-2-yl]piperidin-3-yl}adamantane-1-carboxamide

This compound was prepared using a procedure that was analogous to that described for the synthesis of example 171, steps 1-4. LCMS: (M+H)⁺=438.1.

### Example 184

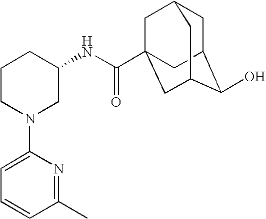

### 4-Hydroxy-N-[(3S)-1-(6-methylpyridin-2-yl)piperidin-3-yl]adamantane-1-carboxamide

This compound was prepared using a procedure that was analogous to that described for the synthesis of example 171, steps 1-4. LCMS: (M+H)⁺=370.2.

### Example 185

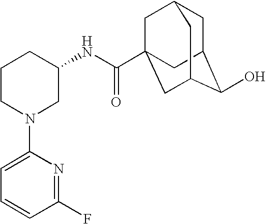

### N-[(3S)-1-(6-Fluoropyridin-2-yl)piperidin-3-yl]-4-hydroxyadamantane-1-carboxamide

This compound was prepared using a procedure that was analogous to that described for the synthesis of example 171, steps 1-4. LCMS: (M+H)⁺=374.1.

### Example 186

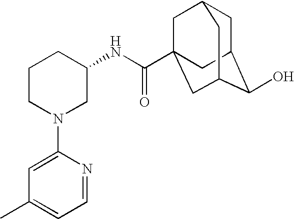

### 4-Hydroxy-N-[(3S)-1-(4-methylpyridin-2-yl)piperidin-3-yl]adamantane-1-carboxamide

This compound was prepared using a procedure that was analogous to that described for the synthesis of example 171, steps 1-4. LCMS: (M+H)⁺=370.2.

### Example 187

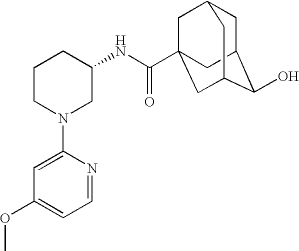

### 4-Hydroxy-N-[(3S)-1-(4-methoxypyridin-2-yl)piperidin-3-yl]adamantane-1-carboxamide

This compound was prepared using a procedure that was analogous to that described for the synthesis of example 171, steps 1-4. LCMS: (M+1)⁺=386.1.

### Example 188

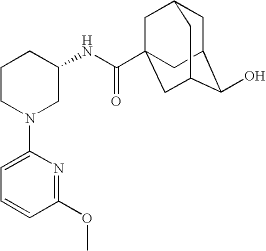

### 4-Hydroxy-N-[(3S)-1-(6-methoxypyridin-2-yl)piperidin-3-yl]adamantane-1-carboxamide

This compound was prepared using a procedure that was analogous to that described for the synthesis of example 171, steps 1-4. LCMS: (M+H)⁺=386.1.

### Example 189

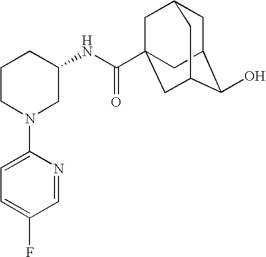

### N-[(3S)-1-(5-Fluoropyridin-2-yl)piperidin-3-yl]-4-hydroxyadamantane-1-carboxamide

This compound was prepared using a procedure that was analogous to that described for the synthesis of example 171, steps 1-4. LCMS: (M+H)⁺=374.1.

### Example 190

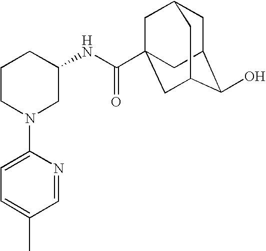

### 4-Hydroxy-N-[(3S)-1-(5-methylpyridin-2-yl)piperidin-3-yl]adamantane-1-carboxamide

This compound was prepared using a procedure that was analogous to that described for the synthesis of example 171, steps 1-4. LCMS: (M+H)⁺=370.1.

### Example 191

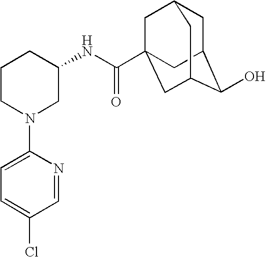

### N-[(3S)-1-(5-Chloropyridin-2-yl)piperidin-3-yl]-4-hydroxyadamantane-1-carboxamide

This compound was prepared using a procedure that was analogous to that described for the synthesis of example 171, steps 1-4. LCMS: (M+H)⁺=390.1/392.1.

### Example 192

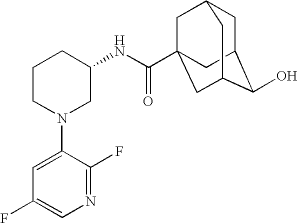

### N-[(3S)-1-(2,5-Difluoropyridin-3-yl)piperidin-3-yl]-4-hydroxyadamantane-1-carboxamide

This compound was prepared using a procedure that was analogous to that described for the synthesis of example 171, steps 1-4. LCMS: (M+H)⁺=392.1.

### Example 193

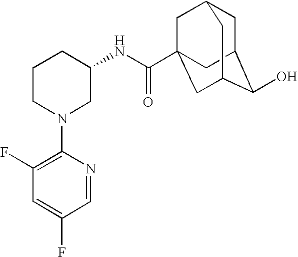

### N-[(3S)-1-(3,5-Difluoropyridin-2-yl)piperidin-3-yl]-4-hydroxyadamantane-1-carboxamide

This compound was prepared using a procedure that was analogous to that described for the synthesis of example 171, steps 1-4. LCMS: (M+H)⁺=392.1.

### Example 194

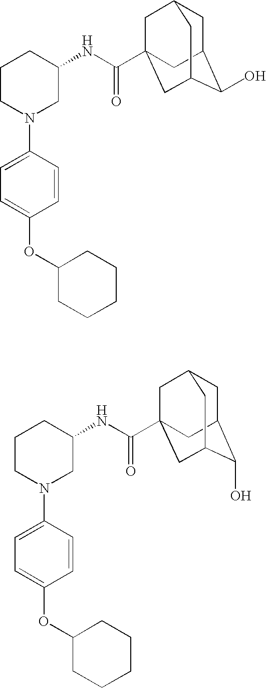

### N-{(3S)-1-[4-(Cyclohexyloxy)phenyl]piperidin-3-yl}-4-hydroxyadamantane-1-carboxamide

This compound was prepared using a procedure that was analogous to that described for the synthesis of example 171, steps 1-4. LCMS: (M+H)⁺=453.2.

### Example 195

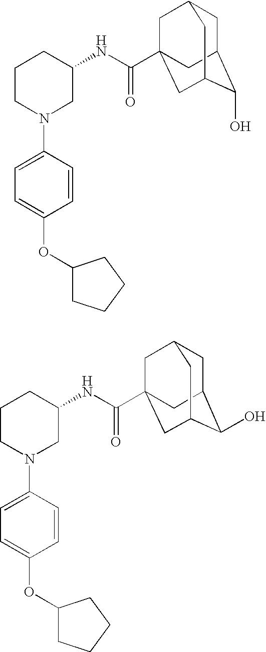

### N-{(3S)-1-[4-(Cyclopentyloxy)phenyl]piperidin-3-yl}-4-hydroxyadamantane-1-carboxamide

This compound was prepared using a procedure that was analogous to that described for the synthesis of example 171, steps 1-4. LCMS: (M+H)⁺=439.3.

### Example 196

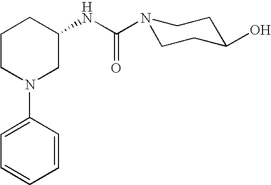

### 4-Hydroxy-N-[(3S)-1-phenylpiperidin-3-yl]piperidine-1-carboxamide

Step 1. 4-Hydroxy-N-[(3S)-piperidin-3-yl]piperidine-1-carboxamide hydrochloride

This compound was prepared using a procedure that was analogous to that described for the synthesis of example 163, steps 1-3. LCMS: (M+H)⁺=228.2.

Step 2. 4-Hydroxy-N-[(3S)-1-phenylpiperidin-3-yl]piperidine-1-carboxamide

Triethylamine (6.0E1 μL, 0.00043 mol) was added to a mixture of 4-hydroxy-N-[(3S)-piperidin-3-yl]piperidine-1-carboxamide hydrochloride (26.7 mg, 0.000101 mol), phenylboronic acid (35.7 mg, 0.000293 mol), cupric acetate (45.6 mg, 0.000251 mol) and 4A molecular sieves (99.3 mg, 0.000443 mol) in tetrahydrofuran (1.0 mL, 0.012 mol). The resulting solution was stirred at rt for 7 h. The crude reaction mixture was purified directly by prep-HPLC to afford the desired product. LCMS: (M+H)⁺=304.2.

### Example 197

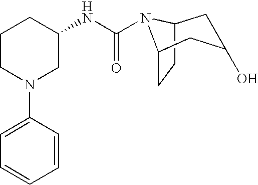

### (1S,5S)-3-Hydroxy-N-[(3S)-1-phenylpiperidin-3-yl]-8-azabicyclo[3.2.1]octane-8-carboxamide

This compound was prepared using a procedure that was analogous to that described for the synthesis of example 196, steps 1-2. LCMS: (M+H)⁺=330.2.

### Example 198

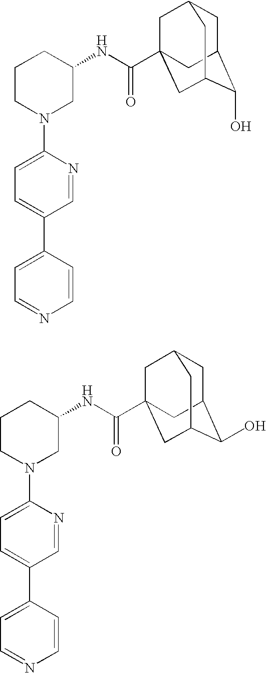

### N-[(3S)-1-(3,4′-bipyridin-6-yl)piperidin-3-yl]-4-hydroxyadamantane-1-carboxamide

Sodium carbonate (10.6 mg, 0.000100 mol) in water (0.10 mL) was added to a mixture of N-[(3S)-1-(5-bromopyridin-2-yl)piperidin-3-yl]-4-hydroxyadamantane-1-carboxamide (21.7 mg, 0.0000500 mol, prepared by using a procedure that was analogous to that described for the synthesis of example 171, steps 1-4) in NMP (0.25 mL), 4-pyridinylboronic acid (7.38 mg, 0.0000600 mol) and tetrakis(triphenylphosphine)palladium(0) (1.7 mg, 0.0000015 mol) in toluene (100.0 μL, 0.0009388 mol) and ethanol (50.000 μL, 8.5633E-4 mol). The resulting mixture was heated at 130° C. for 20 min. Ethyl acetate ( 5 mL) was added and the mixture was washed with water and brine. The organic layer was dried over Na₂SO₄, filtered, and concentrated under reduced pressure. The residue was dissolved in DMF and purified by prep.-HPLC to afford the desired product. LCMS: (M+H)⁺=433.2.

### Example 199

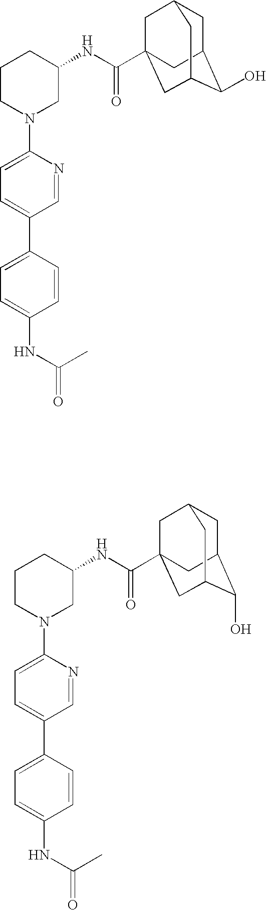

### N-((3S)-1-{5-[4-(Acetylamino)phenyl]pyridin-2-yl}piperidin-3-yl)-4-hydroxyadamantane-1-carboxamide

This compound was prepared using a procedure that was analogous to that described for the synthesis of example 198. LCMS: (M+H)⁺=489.3.

### Example 200

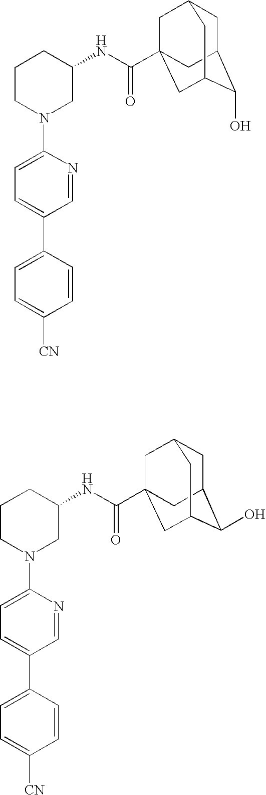

### N-{(3S)-1-[5-(4-cyanophenyl)pyridin-2-yl]piperidin-3-yl}-4-hydroxyadamantane-1-carboxamide

This compound was prepared using a procedure that was analogous to that described for the synthesis of example 198. LCMS: (M+H)⁺=457.2.

### Example 201

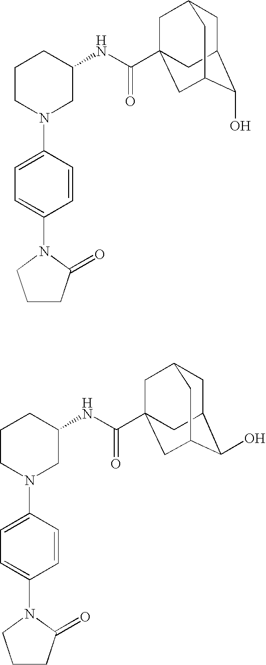

### 4-Hydroxy-N-{(3S)-1-[4-(2-oxopyrrolidin-1-yl)phenyl]piperidin-3-yl}adamantane-1-carboxamide

Copper(I) iodide (0.95 g, 0.0050 mol), 2-pyrrolidinone (570 μL, 0.0075 mol), potassium carbonate (1.4 g, 0.010 mol), N-[(3S)-1-(4-bromophenyl)piperidin-3-yl]-4-hydroxyadamantane-1-carboxamide (0.4 g, 0.001 mol, prepared by using a procedure that was analogous to that described for the synthesis of example 171, steps 1-4), and toluene (5.0 mL, 0.047 mol) were added into a 20-ml vial under an atmosphere of nitrogen. The reaction mixture was stirred at 110° C. for 24 h. The reaction was purified by prep.-BPLC to afford the desired product. LCMS: (M+H)⁺=439.2.

### Example 202

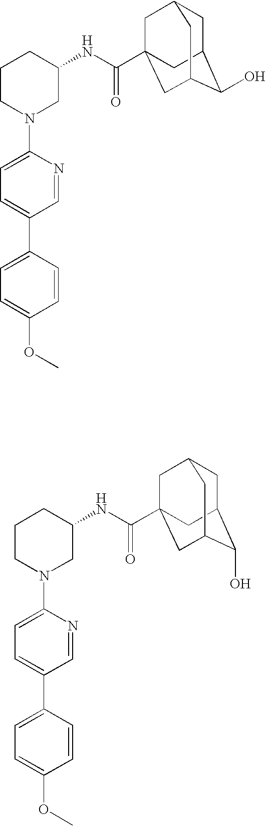

### 4-Hydroxy-N-{(3S)-1-[5-(4-methoxyphenyl)pyridin-2-yl]piperidin-3-yl}adamantane-1-carboxamide

This compound was prepared using a procedure that was analogous to that described for the synthesis of example 198. LCMS: (M+H)⁺=462.3.

### Example 203

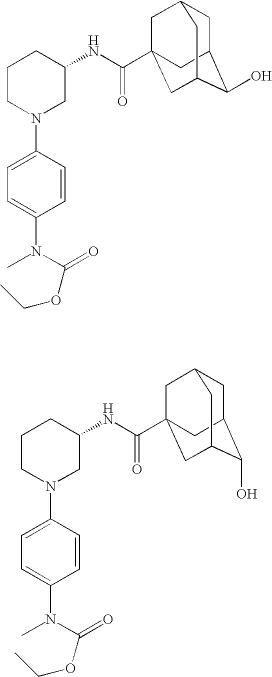

### Ethyl [4-((3S)-3-{[(4-Hydroxy-1-adamantyl)carbonyl]amino}piperidin-1-yl)phenyl]methylcarbamate

This compound was prepared using a procedure that was analogous to that described for the synthesis of example 201. LCMS: (M+H)⁺=456.3.

### Example 204

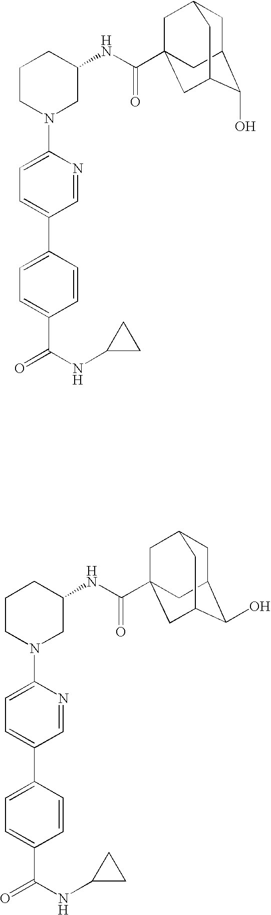

### N-[(3S)-1-(5-{4-[(Cyclopropylamino)carbonyl]phenyl}pyridin-2-yl)piperidin-3-yl]-4-hydroxyadamantane-1-carboxamide

This compound was prepared using a procedure that was analogous to that described for the synthesis of example 198. LCMS: (M+H)⁺=515.3.

### Example 205

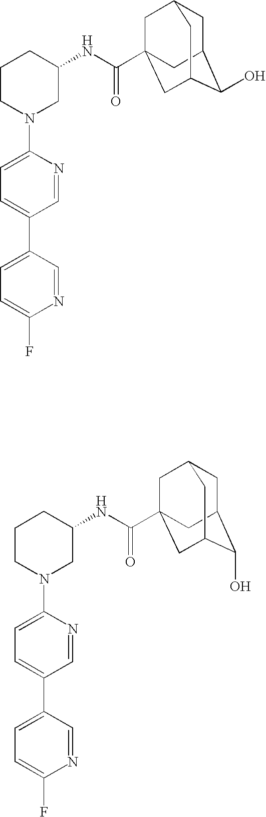

### N-[(3S)-1-(6′-Fluoro-3,3′-bipyridin-6-yl)piperidin-3-yl]-4-hydroxyadamantane-1-carboxamide

This compound was prepared using a procedure that was analogous to that described for the synthesis of example 198. LCMS: (M+H)⁺=451.3.

### Example 206

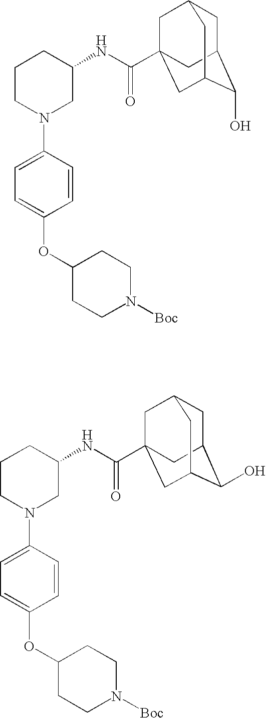

### tert-Butyl 4-[4-((3S)-3-{[(4-hydroxy-1-adamantyl)carbonyl]amino}piperidin-1-yl)phenoxy]piperidine-1-carboxylate

This compound was prepared using a procedure that was analogous to that described for the synthesis of example 141, steps 1-4. LCMS: (M+H)⁺=554.3.

### Example 207

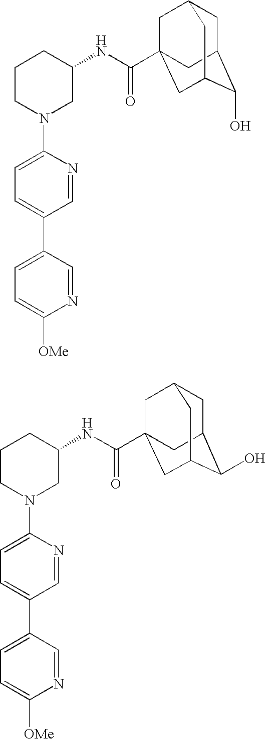

### 4-Hydroxy-N-[(3S)-1-(6′-methoxy-3,3′-bipyridin-6-yl)piperidin-3-yl]adamantane-1-carboxamide

This compound was prepared using a procedure that was analogous to that described for the synthesis of example 198. LCMS: (M+H)⁺=463.3.

### Example 208

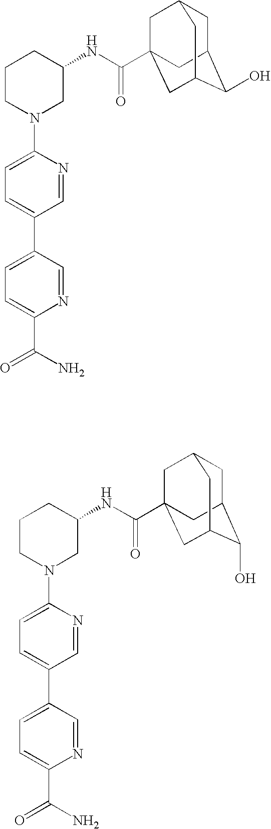

### 6′-((3S)-3-{[(4-Hydroxy-1-adamantyl)carbonyl]amino}piperidin-1-yl)-3,3′-bipyridine-6-carboxamide

This compound was prepared using a procedure that was analogous to that described for the synthesis of example 198. LCMS: (M+H)⁺=476.2.

### Example 209

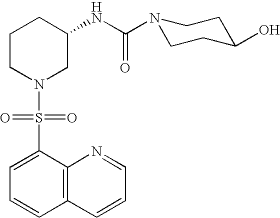

### 4-Hydroxy-N-[(3S)-1-(quinolin-8-ylsulfonyl)piperidin-3-yl]piperidine-1-carboxamide

This compound was prepared using a procedure that was analogous to that described for the synthesis of example 163, steps 1-4. LCMS: (M+H)⁺=419.2.

### Example 210

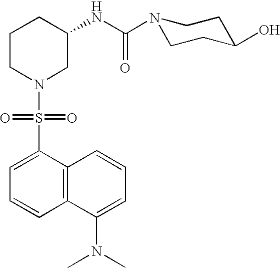

### N-((3S)-1-{[5-(Dimethylamino)-1-naphthyl]sulfonyl}piperidin-3-yl)-4-hydroxypiperidine-1-carboxamide

This compound was prepared using a procedure that was analogous to that described for the synthesis of example 163, steps 1-4. LCMS: (M+H)⁺=461.2.

### Example 211

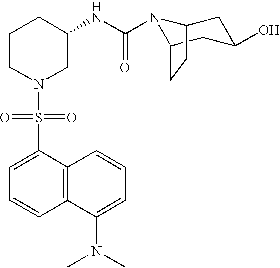

### (3-exo)-N-((3S)-1-{[5-(Dimethylamino)-1-naphthyl]sulfonyl}piperidin-3-yl)-3-hydroxy-8-azabicyclo[3.2.1]octane-8-carboxamide

This compound was prepared using a procedure that was analogous to that described for the synthesis of example 163, steps 1-4. LCMS: (M+H)⁺=487.3.

### Example 212

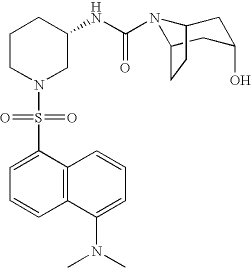

### (3-endo)-N-((3S)-1-{[5-(Dimethylamino)-1-naphthyl]sulfonyl}piperidin-3-yl)-3-hydroxy-8-azabicyclo[3.2.1]octane-8-carboxamide

This compound was prepared using a procedure that was analogous to that described for the synthesis of example 163, steps 1-4. LCMS: (M+H)⁺=487.3.

### Example 213

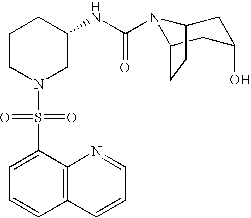

### 3-Hydroxy-N-[(3S)-1-(quinolin-8-ylsulfonyl)piperidin-3-yl]-8-azabicyclo[3.2.1]octane-8-carboxamide

This compound was prepared using a procedure that was analogous to that described for the synthesis of example 163, steps 1-4. LCMS: (M+H)⁺=445.2.

### Example 214

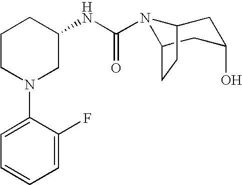

### N-[(3S)-1-(2-Fluorophenyl)piperidin-3-yl]-3-hydroxy-8-azabicyclo[3.2.1]octane-8-carboxamide

This compound was prepared using a procedure that was analogous to that described for the synthesis of example 196. LCMS: (M+H)⁺=348.2.

### Example 215

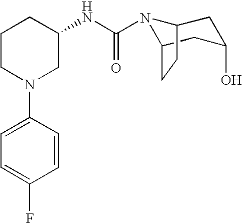

### N-[(3S)-1-(4-Fluorophenyl)piperidin-3-yl]-3-hydroxy-8-azabicyclo[3.2.1]octane-8-carboxamide

This compound was prepared using a procedure that was analogous to that described for the synthesis of example 196. LCMS: (M+H)⁺=348.2.

### Example 216

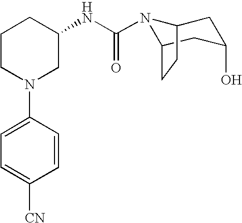

### (3-endo)-N-[(3S)-1-(4-Cyanophenyl)piperidin-3-yl]-3-hydroxy-8-azabicyclo[3.2.1]octane-8-carboxamide

This compound was prepared using a procedure that was analogous to that described for the synthesis of example 196. LCMS: (M+H)⁺=355.3.

### Example 217

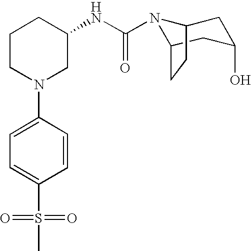

### (3-endo)-3-Hydroxy-N-{(3S)-1-[4-(methylsulfonyl)phenyl]piperidin-3-yl}-8-azabicyclo[3.2.1]octane-8-carboxamide

This compound was prepared using a procedure that was analogous to that described for the synthesis of example 196. LCMS: (M+H)⁺=408.2.

### Example 218

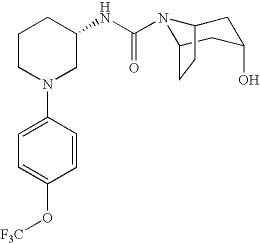

### (3-endo)-3-Hydroxy-N-{(3S)-1-[4-(trifluoromethoxy)phenyl]piperidin-3-yl}-8-azabicyclo[3.2.1]octane-8-carboxamide

This compound was prepared using a procedure that was analogous to that described for the synthesis of example 196. LCMS: (M+H)⁺=414.2.

### Example 219

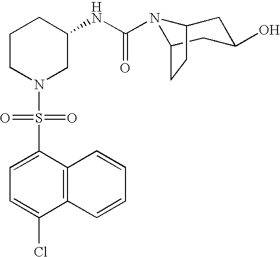

### N-{(3S)-1-[(4-Chloro-1-naphthyl)sulfonyl]piperidin-3-yl}-4-hydroxypiperidine-1-carboxamide

This compound was prepared using a procedure that was analogous to that described for the synthesis of example 123, steps 3 & 4, followed by a procedure that was analogous to that described for the synthesis of example 9, steps 2 & 3. LCMS: (M+H)⁺=452.2.

### Example 220

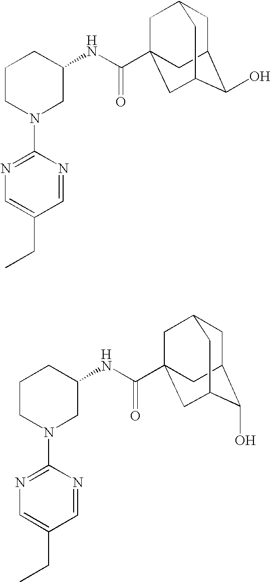

### N-[(3S)-1-(5-Ethylpyrimidin-2-yl)piperidin-3-yl]-4-hydroxyadamantane-1-carboxamide

This compound was prepared using a procedure that was analogous to that described for the synthesis of example 141, steps 1-4. LCMS: (M+H)⁺=385.3.

### Example 221

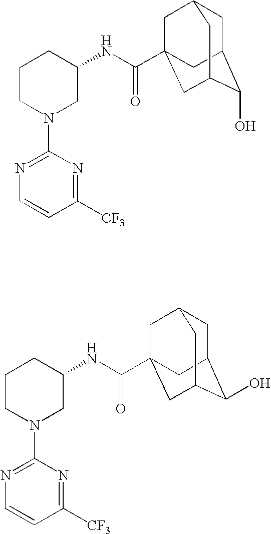

### 4-Hydroxy-N-{(3S)-1-[4-(trifluoromethyl)pyrimidin-2-yl]piperidin-3-yl}adamantane-1-carboxamide

This compound was prepared using a procedure that was analogous to that described for the synthesis of example 141, steps 1-4. LCMS: (M+H)⁺=425.2.

### Example 222

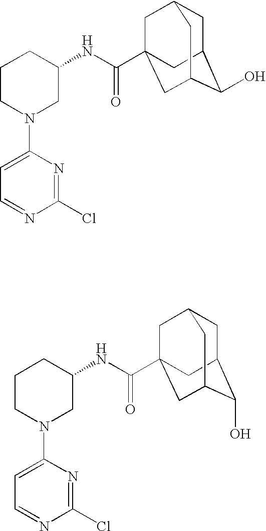

### N-[(3S)-1-(2-Chloropyrimidin-4-yl)piperidin-3-yl]-4-hydroxyadamantane-1-carboxamide

This compound was prepared using a procedure that was analogous to that described for the synthesis of example 141, steps 1-4. LCMS: (M+H)⁺=391.2/393.2.

### Example 223

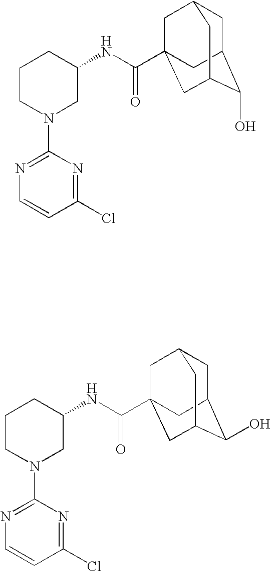

### N-[(3S)-1-(4-Chloropyrimidin-2-yl)piperidin-3-yl]-4-hydroxyadamantane-1-carboxamide

This compound was prepared using a procedure that was analogous to that described for the synthesis of example 141, steps 1-4. LCMS: (M+H)⁺=391.2/393.2.

### Example 224

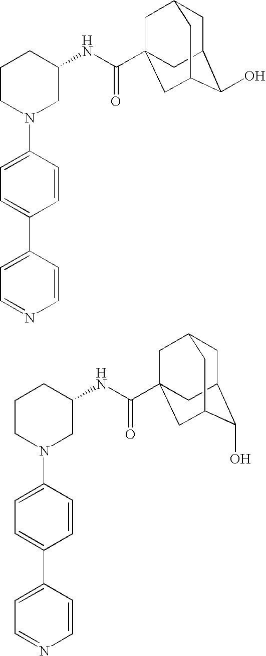

### 4-Hydroxy-N-[(3S)-1-(4-pyridin-4-ylphenyl)piperidin-3-yl]adamantane-1-carboxamide

This compound was prepared using a procedure that was analogous to that described for the synthesis of example 171, steps 1-4. LCMS: (M+H)⁺=432.3.

### Example 225

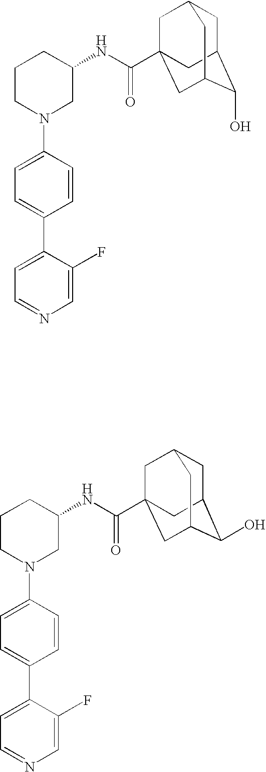

### N-{(3S)-1-[4-(3-Fluoropyridin-4-yl)phenyl]piperidin-3-yl}-4-hydroxyadamantane-1-carboxamide

This compound was prepared using a procedure that was analogous to that described for the synthesis of example 171, steps 1-4. LCMS: (M+H)⁺=450.3.

### Example 226

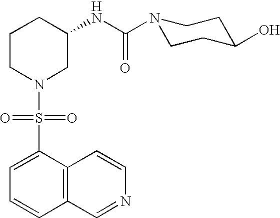

### 4-Hydroxy-N-[(3S)-1-(isoquinolin-5-ylsulfonyl)piperidin-3-yl]piperidine-1-carboxamide

This compound was prepared using a procedure that was analogous to that described for the synthesis of example 163, steps 1-4. LCMS: (M+H)⁺=419.2.

### Example 227

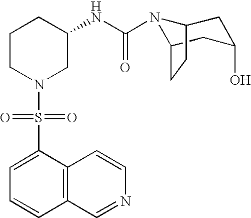

### (3-endo)-3-Hydroxy-N-[(3S)-1-(isoquinolin-5-ylsulfonyl)piperidin-3-yl]-8-azabicyclo[3.2.1]octane-8-carboxamide

This compound was prepared using a procedure that was analogous to that described for the synthesis of example 163, steps 1-4. LCMS: (M+H)⁺=445.2.

### Example 228

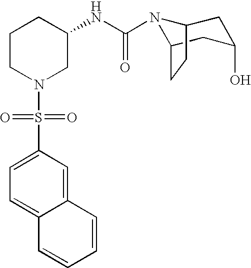

### (3-endo)-3-Hydroxy-N-[(3S)-1-(2-naphthylsulfonyl)piperidin-3-yl]-8-azabicyclo[3.2.1]octane-8-carboxamide

This compound was prepared using a procedure that was analogous to that described for the synthesis of example 163, steps 1-4. LCMS: (M+H)⁺=444.2.

### Example 229

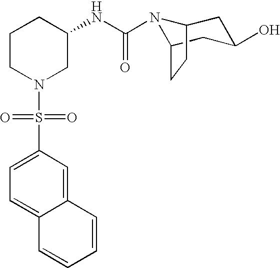

### (3-exo)-3-hydroxy-N-[(3S)-1-(2-naphthylsulfonyl)piperidin-3-yl]-8-azabicyclo[3.2.1]octane-8-carboxamide

This compound was prepared using a procedure that was analogous to that described for the synthesis of example 163, steps 1-4. LCMS: (M+H)⁺=444.2.

### Example 230

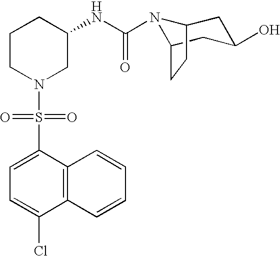

### (3-exo)-N-{(3S)-1-[(4-Chloro-1-naphthyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+H)⁺=478.1/480.2.

### Example 231

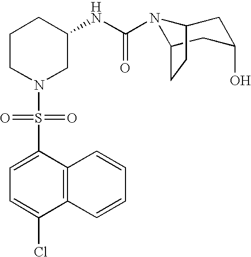

### (3-endo)-N-{(3S)-1-[(4-Chloro-1-naphthyl)sulfonyl]piperidin-3-yl}-3-hydroxy-8-azabicyclo[3.2.1]octane-8-carboxamide

This compound was prepared using a procedure that was analogous to that described for the synthesis of example 163, steps 1-4. LCMS: (M+H)⁺=478.1/480.2.

### Example 232

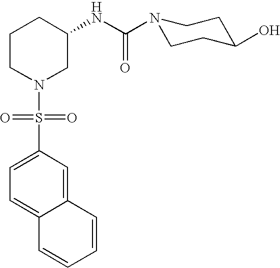

### 4-hydroxy-N-[(3S)-1-(2-naphthylsulfonyl)piperidin-3-yl]piperidine-1-carboxamide

This compound was prepared using a procedure that was analogous to that described for the synthesis of example 163, steps 1-4. LCMS: (M+H)⁺=418.2.

### Example 233

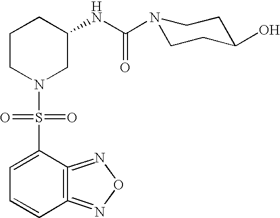

### N-[(3S)-1-(2,1,3-Benzoxadiazol-4-ylsulfonyl)piperidin-3-yl]-4-hydroxypiperidine-1-carboxamide

This compound was prepared using a procedure that was analogous to that described for the synthesis of example 163, steps 1-4. LCMS: (M+H)⁺=410.2.

### Example 234

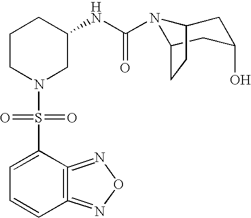

### (3-endo)-N-[(3S)-1-(2,1,3-Benzoxadiazol-4-ylsulfonyl)piperidin-3-yl]-3-hydroxy-8-azabicyclo[3.2.1]octane-8-carboxamide

This compound was prepared using a procedure that was analogous to that described for the synthesis of example 163, steps 1-4. LCMS: (M+H)⁺=436.2.

### Example 235

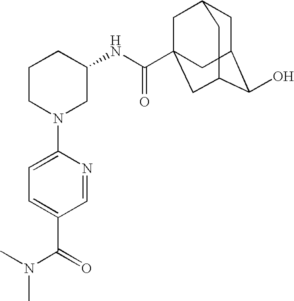

### 6-((3S)-3-{[(4-Hydroxy-1-adamantyl)carbonyl]amino}piperidin-1-yl)-N,N-dimethylnicotinamide

A mixture of 4-hydroxy-N-[(3S)-piperidin-3-yl]adamantane-1-carboxamide (13.9 mg, 0.0000500 mol, prepared by using a procedure that was analogous to that described for the synthesis of example 141, steps 1-3), 6-chloro-N,N-dimethylnicotinamide (13.8 mg, 0.0000750 mol) and N,N-diisopropylethylamine (19.4 mg, 0.000150 mol) in N,N-dimethylformamide (0.500 mL, 0.00646 mol) was irradiated under microwave at 120° C. for 10 min. The mixture was adjusted with TFA to pH=2.0 and was diluted with methanol (0.8 mL). The resulting solution was purified by prep.-HPLC to give the desired product. LCMS: (M+H)⁺=427.2.

### Example 236

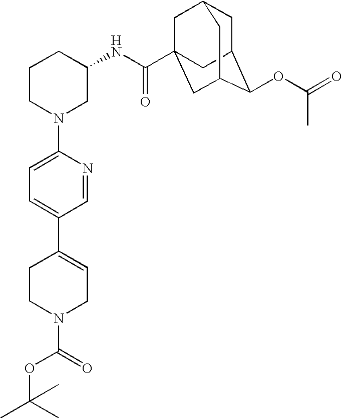

### tert-Butyl 6-[(3S)-3-({[4-(acetyloxy)-1-adamantyl]carbonyl}amino)piperidin-1-yl]-3′,6′-dihydro-3,4′-bipyridine-1′(2′H)-carboxylate

This compound was prepared using a procedure that was analogous to that described for the synthesis of example 198. LCMS: (M+H)⁺=579.3.

### Example 237

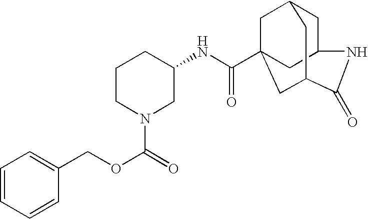

### Benzyl (3S)-3-{[(5-oxo-4-azatricyclo[4.3.1.1(3,8)]undec-1-yl)carbonyl]amino}piperidine-1-carboxylate

Step 1. Benzyl (3S)-3-[(tert-butoxycarbonyl)amino]piperidine-1-carboxylate

This compound was prepared using a procedure that was analogous to that described for the synthesis of example 123, step 1. LCMS: (M+H)⁺=335.2.

Step 2. Benzyl (3S)-3-aminopiperidine-1-carboxylate hydrochloride

This compound was prepared using a procedure that was analogous to that described for the synthesis of example 141, step 3. LCMS: (M+H)⁺=271.3.

Step 3. Benzyl (3S)-3-{[(4-oxo-1-adamantyl)carbonyl]amino}piperidine-1-carboxylate

This compound was prepared using a procedure that was analogous to that described for the synthesis of example 141, step 1. LCMS: (M+H)⁺=411.2.

Step 4. Benzyl (3S)-3-({[4-(hydroxyimino)-1-adamantyl]carbonyl}amino)piperidine-1-carboxylate

Benzyl (3S)-3-{[(4-oxo-1-adamantyl)carbonyl]amino}piperidine-1-carboxylate (82.1 mg, 0.000200 mol) in methanol (1.0 mL) was treated with hydroxylamine (50.0 μL, 0.000817 mol) and the mixture was stirred at rt overnight. The solvent was evaporated in-vacuo to afford the desired product, which was used directly in the next step without further purification.

Step 5. Benzyl (3S)-3-{[(5-oxo-4-azatricyclo[4.3.1.1(3,8)]undec-1-yl)carbonyl]amino}piperidine-1-carboxylate

Benzyl (3S)-3-({[4-(hydroxyimino)-1-adamantyl]carbonyl}amino)piperidine-1-carboxylate (0.083 g, 0.00020 mol) was treated with concentrated HCl (0.3 mL) at rt for 1 h. The mixture was neutralized with 1N NaOH to pH=3 and diluted with DMF (3.0 mL). The resulting mixture was purified by prep.-HPLC to give the desired product. LCMS: (M+H)⁺=426.2.

### Example 238

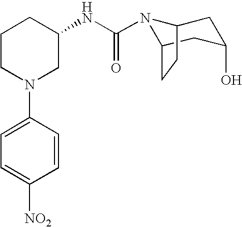

### (3-endo)-3-Hydroxy-N-(3S)-1-(4-nitrophenyl)piperidin-3-yl]-8-azabicyclo[3.2.1]octane-8-carboxamide

Step 1: (3S)-1-(4-nitrophenyl)piperidin-3-amine

To a stirred solution of tert-butyl (3S)-piperidin-3-ylcarbamate (2.50 g, 0.0125 mol) in N,N-dimethylformamide (15.00 mL, 0.1937 mol) was added 4-fluoronitrobenzene (2.29 g, 0.0162 mol), potassium carbonate (2.59 g, 0.0187 mol). After stirring the reaction mixture at 90° C. for 13 h, the reaction mixture was cooled to ambient temperature and the mixture was diluted with EtOAc, washed with water, and brine. The organic layers were dried and concentrated in-vacuo and the resultant residue was used in the next step without further purification. LCMS (M+H)⁺ 322.2. The crude material prepared above was treated with 50 mL of TFA at rt for 1 h. The volatiles were removed in-vacuo and the residue was diluted with methylene chloride and washed with 1 N NaOH. The organic layers were combined, washed with water, brine, dried, and evaporated to dryness. LCMS (M+H)⁺ 222.2.

Step 2: (3-endo)-3-Hydroxy-N-[(3S)-2-(4-nitrophenyl)piperidin-3-yl]-8-azabicyclo[3.2.1]octane-8-carboxamide

The title compound was prepared using a procedure that was analogous to that described for the synthesis of example 123, steps 3 and 4. LCMS: (M+H)⁺=375.2.

### Example 239

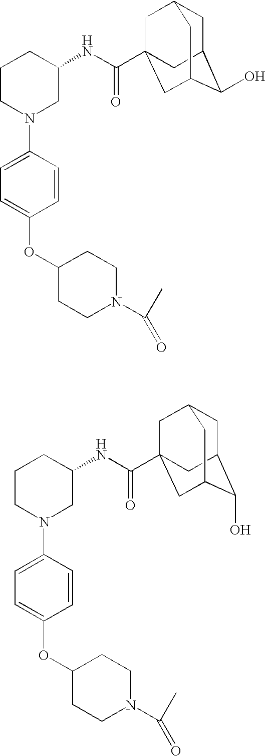

### N-((3S)-1-{4-[(1-Acetylpiperidin-4-yl)oxy]phenyl}piperidin-3-yl)-4-hydroxyadamantane-1-carboxamide

The title compound was prepared using a procedure that was analogous to that described for the synthesis of example 171, steps 1-4, starting from 4-hydroxy-N-[(3S)-piperidin-3-yl]adamantane-1-carboxamide and tert-butyl 4-(4-chlorophenoxy)piperidine-1-carboxylate to afford tert-butyl 4-[4-((3S)-3-{[(4-hydroxy-1-adamantyl)carbonyl]amino}piperidin-1-yl)phenoxy]piperidine-1-carboxylate, which was subsequently deprotected and acylated using the protocol outlined in example 1 step 2. LCMS: (M+H)⁺=496.3.

### Example 240

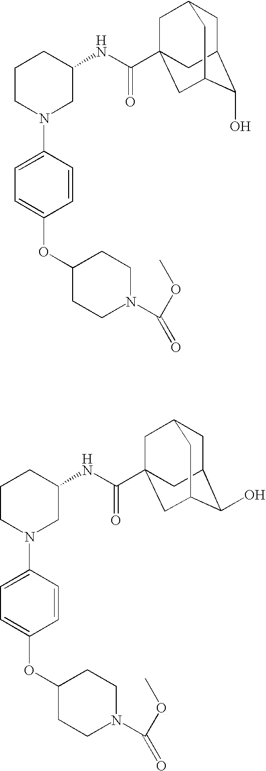

### Methyl 4-[4-((3S)-3-{[(4-hydroxy-1-adamantyl)carbonyl]amino}piperidin-1-yl)phenoxy]piperidine-1-carboxylate

The title compound was prepared using a procedure that was analogous to that described for the synthesis of example 239. LCMS: (M+H)⁺=512.3.

### Example 241

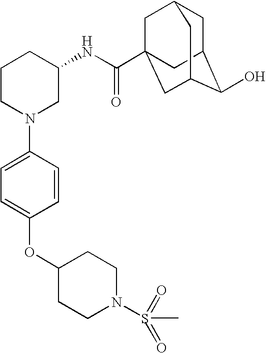

### 4-Hydroxy-N-[(3S)-1-(4-{[1-(methylsulfonyl)piperidin-4-yl]oxy}phenyl)piperidin-3-yl]adamantane-1-carboxamide

The title compound was prepared using a procedure that was analogous to that described for the synthesis of example 239. LCMS: (M+H)⁺=532.3.

### Example 242

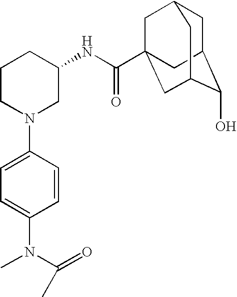

### N-((3S)-1-{4-[Acetyl(methyl)amino]phenyl}piperidin-3-yl)-4-hydroxyadamantane-1-carboxamide

The title compound was prepared using a procedure that was analogous to that described for the synthesis of example 171, steps 1-4. LCMS: (M+H)⁺=426.3.

### Example 243

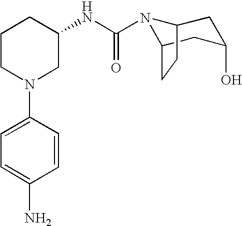

### (3-endo)-N-[(3S)-1-(4-Aminophenyl)piperidin-3-yl]-3-hydroxy-8-azabicyclo[3.2.1]octane-8-carboxamide

A mixture of (3-endo)-3-hydroxy-N-[(3S)-1-(4-nitrophenyl)piperidin-3-yl]-8-azabicyclo[3.2.1]octane-8-carboxamide (1.86 g, 0.00497 mol, prepared as example 238) in 50 mL of MeOH was hydrogenated in the presence of 10% Pd/C under balloon pressure of hydrogen overnight. The catalyst was filtered off and the filtrate was concentrated in-vacuo. LCMS: (M+H)⁺=345.3.

### Example 244

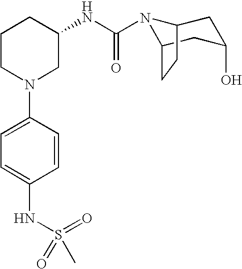

### (3-endo)-3-Hydroxy-N-((3S)-1-{4-[(methylsulfonyl)amino]phenyl}piperidin-3-yl)-8-azabicyclo[3.2.1]octane-8-carboxamide

To a mixture of (3-endo)-N-[(3S)-1-(4-aminophenyl)piperidin-3-yl]-3-hydroxy-8-azabicyclo[3.2.1]octane-8-carboxamide (30.0 mg, 0.0000871 mol, prepared as example 243) and 4-dimethylaminopyridine (16.0 mg, 0.000131 mol) in methylene chloride (0.30 mL, 0.0047 mol) was added methanesulfonyl chloride (0.00843 mL, 0.000109 mol). The mixture was stirred at rt for 1 h. After removal of the volatiles in-vacuo the residue was diluted with water and ACN and purified on RP-HPLC to give the product. LCMS (M+H)⁺ 423.2.

### Example 245

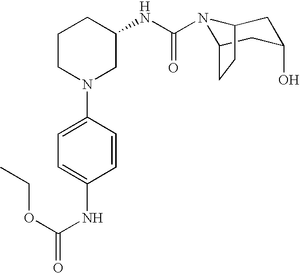

### Ethyl {4-[(3S)-3-({[(3-endo)-3-hydroxy-8-azabicyclo[3.2.1]oct-8-yl]carbonyl}amino)piperidin-1-yl]phenyl}carbamate

To a mixture of (3-endo)-N-[(3S)-1-(4-aminophenyl)piperidin-3-yl]-3-hydroxy-8-azabicyclo[3.2.1]octane-8-carboxamide (30.0 mg, 0.0000871 mol, prepared as example 243) in methylene chloride (0.30 mL, 0.0047 mol) was added 1.0 M of sodium hydroxide in water (0.1306 mL) followed by ethyl chloroformate (0.0104 mL, 0.000109 mol). The reaction was stirred at rt for 1 h. After removal of the volatiles in-vacuo, the residue was neutralized with diluted TFA and purified on RP-HPLC to give the desired product. LCMS (M+H) 417.3.

### Example 246

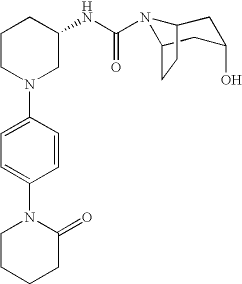

### (3-endo)-3-Hydroxy-N-{(3S)-1-[4-(2-oxopiperidin-1-yl)phenyl]piperidin-3-yl}-8-azabicyclo[3.2.1]octane-8-carboxamide

To a mixture of (3-endo)-N-[(3S)-1-(4-aminophenyl)piperidin-3-yl]-3-hydroxy-8-azabicyclo[3.2.1]octane-8-carboxamide (30.0 mg, 0.0000871 mol, prepared as example 243) and 4-dimethylaminopyridine (15.96 mg, 0.0001306 mol) in tetrahydrofuran (0.80 mL, 0.0099 mol) was added 5-bromovaleryl chloride (0.0146 mL, 0.000109 mol). The reaction was stirred at rt for 1 h to afford the acylated product, which was detected by LCMS (M+H)⁺ 507.2. To the reaction mixture was added 1.0 M of potassium tert-butoxide in tetrahydrofuran (0.261 mL). After stirring for 2 h the volatiles were removed in-vacuo and the residue was neutralized by diluted TFA and purified on RP-HPLC to afford the desired product. LCMS (M+H)⁺ 427.3.

### Example 247

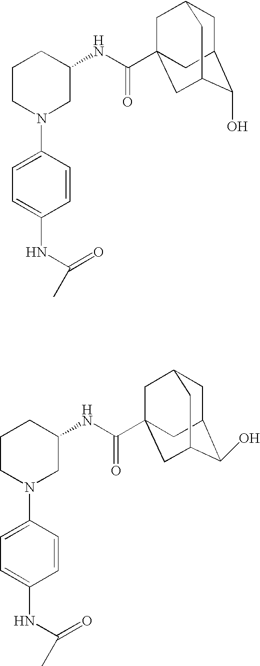

### N-{(3S)-1-[4-(Acetylamino)phenyl]piperidin-3-yl}-4-hydroxyadamantane-1-carboxamide

The title compound was prepared using a procedure that was analogous to that described for the synthesis of example 171, steps 1-4. LCMS: (M+H)⁺=412.2.

### Example 248

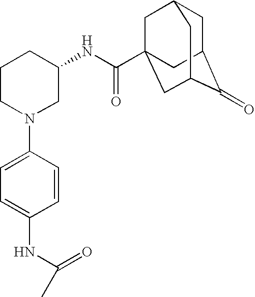

### N-{(3S)-1-[4-(Acetylamino)phenyl]piperidin-3-yl}-4-oxoadamantane-1-carboxamide

The title compound was prepared using a procedure that was analogous to that described for the synthesis of example 171, steps 1, 3 and 4. LCMS: (M+H)⁺=410.2.

### Example 249

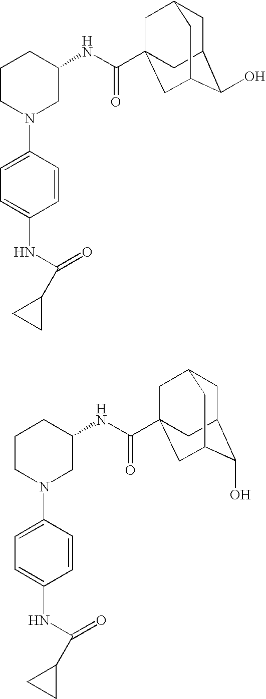

### N-((3S)-1-{4-[(Cyclopropylcarbonyl)amino]phenyl}piperidin-3-yl)-4-hydroxyadamantane-1-carboxamide

The title compound was prepared using a procedure that was analogous to that described for the synthesis of example 171, steps 1-4. LCMS: (M+H)⁺=438.3.

### Example 250

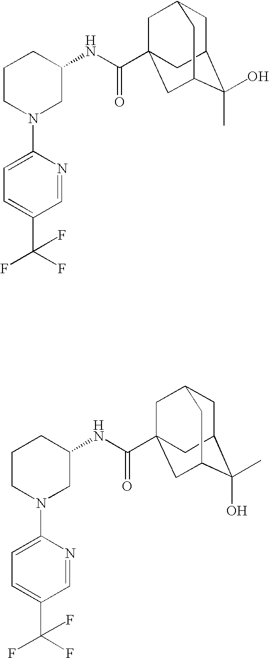

### 4-Hydroxy-4-methyl-N-{(3S)-1-[5-(trifluoromethyl)pyridin-2-yl]piperidin-3-yl}adamantane-1-carboxamide

Step 1: Benzyl (3S)-3-{[(4-hydroxy-4-methyl-1-adamantyl)carbonyl]amino}piperidine-1-carboxylate

Benzyl (3S)-3-{[(4-oxo-1-adamantyl)carbonyl]amino}piperidine-1-carboxylate (41.0 mg, 0.0000999 mol, prepared as the product from step 1 in the synthesis of example 171) in THF (2.0 mL) was cooled with a dry-ice bath to −78° C. To the cooled solution was added methyllithium (0.15 mL, 0.0050 mol). After stirring for 30 min., the reaction was quenched with a saturated ammonium chloride solution and was extracted with ethyl acetate. The organic layer was dried over Na₂SO₄, filtered, and concentrated under reduced pressure.

Step 2: 4-Hydroxy-4-methyl-N-[(3S)-piperidin-3-yl]adamantane-1-carboxamide

This compound was prepared using a procedure that was analogous to that described for the synthesis of example 171, steps 3. LCMS: (M+1)⁺=293.3.

Step 3: 4-Hydroxy-4-methyl-N-{(3S)-1-[5-(trifluoromethyl)pyridin-2-yl]piperidin-3-yl}adamantane-1-carboxamide

A mixture of 4-hydroxy-4-methyl-N-[(3S)-piperidin-3-yl]adamantane-1-carboxamide (20.6 mg, 0.0000704 mol), 2-chloro-5-(trifluoromethyl)pyridine (19.2 mg, 0.000106 mol) and N,N-diisopropylethylamine (35 uL, 0.00020 mol) in N,N-dimethylformamide (0.705 mL, 0.00911 mol) was irradiated under microwave at 150° C. for 20 min. The mixture was adjusted with TFA to pH=2.0 and was diluted with methanol (0.8 mL). The resulting solution was purified by prep.-HPLC to give the desired product. LCMS: (M+H)⁺=438.3.

### Example 251

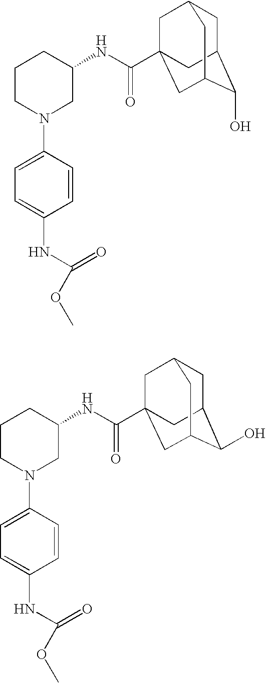

### Methyl [4-((3S)-3-{[(4-hydroxy-1-adamantyl)carbonyl]amino}piperidin-1-yl)phenyl]carbamate

The title compound was prepared using a procedure that was analogous to that described for the synthesis of example 171, steps 1-4. LCMS: (M+H)⁺=428.3.

### Example 252

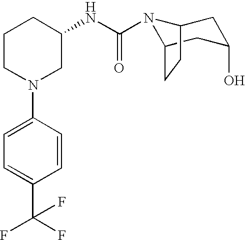

### (3-endo)-3-Hydroxy-N-{(3S)-1-[4-(trifluoromethyl)phenyl]piperidin-3-yl}-8-azabicyclo[3.2.1]octane-8-carboxamide

The title compound was prepared using a procedure that was analogous to that described for the synthesis of example 196, steps 1 and 2. LCMS: (M+H)⁺=398.2.

### Example 253

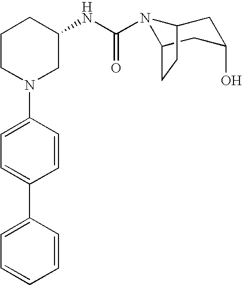

### (3-endo)-N-[(3S)-1-Biphenyl-4-ylpiperidin-3-yl]-3-hydroxy-8-azabicyclo[3.2.1]octane-8-carboxamide

The title compound was prepared using a procedure that was analogous to that described for the synthesis of example 196, steps 1 and 2. LCMS: (M+H)⁺=406.3.

### Example 254

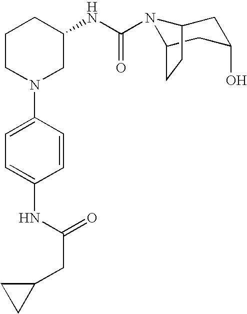

### (3-endo)-N-((3S)-1-{4-[(Cyclopropylacetyl)amino]phenyl}piperidin-3-yl)-3-hydroxy-8-azabicyclo[3.2.1]octane-8-carboxamide

To a mixture of (3-endo)-N-[(3S)-1-(4-aminophenyl)piperidin-3-yl]-3-hydroxy-8-azabicyclo[3.2.1]octane-8-carboxamide (30.0 mg, 0.0000871 mol, prepared as example 243) and cyclopropaneacetic acid (10.9 mg, 0.000109 mol) in N,N-dimethylformamide (0.30 mL, 0.0039 mol) was added benzotriazol-1-yloxytris(dimethylamino)phosphonium hexafluorophosphate (57.8 mg, 0.000131 mol). After stirring at rt for 10 min., N,N-diisopropylethylamine (0.0303 mL, 0.000174 mol) was added and the reaction mixture was stirred at rt for an additional hour. The crude mixture was diluted with ACN and water and was purified on RP-HPLC to give the desired product. LCMS (M+H)⁺ 427.3.

### Example 255

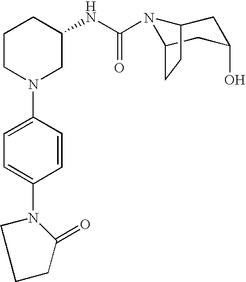

### (3-endo)-3-Hydroxy-N-{(3S)-1-[(2-oxopyrrolidin-1-yl)phenyl]piperidin-3-yl}-8-azabicyclo[3.2.1]octane-8-carboxamide

To a mixture of (3-endo)-N-[(3S)-1-(4-aminophenyl)piperidin-3-yl]-3-hydroxy-8-azabicyclo[3.2.1]octane-8-carboxamide (30.0 mg, 0.0000871 mol, prepared as example 243) and 4-dimethylaminopyridine (15.96 mg, 0.0001306 mol) in tetrahydrofuran (0.80 mL, 0.0099 mol) was added 4-bromobutanoyl chloride, (0.0126 mL, 0.000109 mol). After stirring the reaction mixture at rt for 1 h, 1.0 M of potassium tert-butoxide in tetrahydrofuran (0.348 mL) was added and stirring was continued at rt for 2 h. The volatiles were removed in-vacuo and the residue was neutralized with diluted TFA and purified on RP-HPLC to give the product. LCMS (M+H)⁺ 427.3.

### Example 256

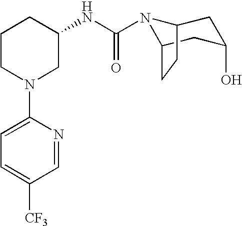

### (3-endo)-3-Hydroxy-N-{(3S)-1-[5-(trifluoromethyl)pyridin-2-yl]piperidin-3-yl}-8-azabicyclo[3.2.1]octane-8-carboxamide

A mixture of (3-endo)-3-hydroxy-N-[(3S)-piperidin-3-yl]-8-azabicyclo[3.2.1]octane-8-carboxamide hydrochloride (15.3 mg, 0.0000528 mol; prepared as example 163, steps 1-3), N,N-diisopropylethylamine (55 μL, 0.00032 mol), and 2-chloro-5-(trifluoromethyl)pyridine (15.0 mg, 0.0000826 mol) in N-methylpyrrolidinone (0.75 mL, 0.0078 mol) was irradiated with microwaves at 150° C. for 15 min. LCMS (M+H)⁺ 399.2.

### Example 257

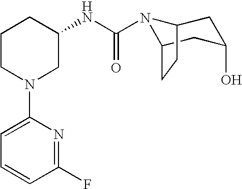

### (3-endo)-N-[(3S)-1-(6-Fluoropyridin-2-yl)piperidin-3-yl]-3-hydroxy-8-azabicyclo[3.2.1]octane-8-carboxamide

The title compound was prepared using a procedure that was analogous to that described for the synthesis of example 256. LCMS: (M+H)⁺=349.3.

### Example A

### Enzymatic Assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Test compounds having an IC₅₀ value less than about 20 μM according to this assay were considered active.

### Example B

### Cell-based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/ml recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 20 μM according to this assay were considered active.

### Example C

### Cellular Assay to Evaluate MR Antagonism

Assays for MR antagonism were performed essentially as described (Jausons-Loffreda et al. J Biolumin and Chemilumin, 1994, 9: 217-221). Briefly, HEK293/MSR cells (Invitrogen Corp.) were co-transfected with three plasmids: 1) one designed to express a fusion protein of the GAL4 DNA binding domain and the mineralocorticoid receptor ligand binding domain, 2) one containing the GAL4 upstream activation sequence positioned upstream of a firefly luciferase reporter gene (pFR-LUC, Stratagene, Inc.), and 3) one containing the Renilla luciferase reporter gene cloned downstream of a thymidine kinase promoter (Promega). Transfections were performed using the FuGENE6 reagent (Roche). Transfected cells were ready for use in subsequent assays 24 hours post-transfection.

In order to evaluate a compound's ability to antagonize the MR, test compounds were diluted in cell culture medium (E-MEM, 10% charcoal-stripped FBS, 2 mM L-glutamine) supplemented with 1 nM aldosterone and applied to the transfected cells for 16-18 hours. After the incubation of the cells with the test compound and aldosterone, the activity of firefly luciferase (indicative of MR agonism by aldosterone) and Renilla luciferase (normalization control) were determined using the Dual-Glo Luciferae Assay System (Promega). Antagonism of the mineralocorticoid receptor was determined by monitoring the ability of a test compound to attenuate the aldosterone-induced firefly luciferase activity.

Compounds having an IC₅₀ of 200 μM or less were considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula Ia: 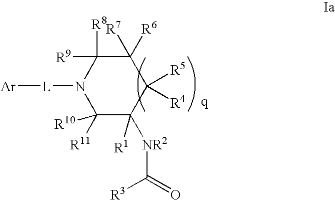
or pharmaceutically acceptable salt or prodrug thereof, wherein:
L is absent, S(O)₂, S(O), S, C(O), C(O)O, C(O)O-(C₁₋₃ alkylene), or C(O)NR^{L};
Ar is aryl or heteroaryl, each optionally substituted by 1, 2, 3, 4 or 5 -W-X-Y-Z;
R^{L} is H or C₁₋₆ alkyl;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R² is H, C₁₋₆ alkyl, arylalkyl, heteroarylalkyl, cycloalkyl, cycloalkylalkyl, heterocycloalkyl or heterocycloalkylalkyl, each optionally substituted by 1, 2 or 3 R¹⁴;
R³ is H, C₁₋₆ alkyl, aryl, cycloalkyl, heteroaryl, heterocycloalkyl, each optionally substituted by 1, 2 or 3-W′-X′-Y′-Z′;
or R³ is NR^{3a}R^{3b};
R^{3a} and R^{3b} are each, independently, H, C₁₋₆ alkyl, aryl, cycloalkyl, heteroaryl, heterocycloalkyl, each optionally substituted by 1, 2 or 3-W′-X′-Y′-Z′;
or R^{3a} and R^{3b} together with the N atom to which they are attached form a 4-14 membered heterocycloalkyl group which is optionally substituted by 1, 2 or 3-W′-X′-Y′-Z′;
R⁴, R⁵, R⁶, R⁷, R⁸, R⁹, R¹⁰ and R¹¹ are each, independently, H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
or R¹ and R² together with the carbon and nitrogen atoms to which they are attached form a 3-14 membered heterocycloalkyl group which is optionally substituted by 1, 2 or 3 R¹⁴;
or R¹ and R³ together with the carbon atoms to which they are attached and the intervening —NR²CO— moiety form a 4-14 membered heterocycloalkyl group which is optionally substituted by 1, 2 or 3 R¹⁴;
or R² and R³ together with the carbon and nitrogen atoms to which they are attached form a 3-14 membered heterocycloalkyl group which is optionally substituted by 1, 2 or 3 R¹⁴;
or R⁴ and R⁵ together with the carbon atom to which they are attached form a 3-14 membered cycloalkyl or heterocycloalkyl group which is optionally substituted by 1, 2 or 3 R¹⁴;
or R⁶ and R⁷ together with the carbon atom to which they are attached form a 3-14 membered cycloalkyl or heterocycloalkyl group which is optionally substituted by 1, 2 or 3 R¹⁴;
or R⁸ and R⁹ together with the carbon atom to which they are attached form a 3-14 membered cycloalkyl or heterocycloalkyl group which is optionally substituted by 1, 2 or 3 R¹⁴;
or R¹⁰ and R¹¹ together with the carbon atom to which they are attached form a 3-14 membered cycloalkyl or heterocycloalkyl group which is optionally substituted by 1, 2 or 3 R¹⁴;
or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atom to which they are attached form a 3-7 membered fused cycloalkyl group or 3-7 membered fused heterocycloalkyl group which is optionally substituted by 1, 2 or 3 R¹⁴;
R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two -W-X-Y-Z attached to the same atom optionally form a 3-14 membered cycloalkylk or 3-14 membered heterocycloalkyl group optionally substituted by 1, 2 or 3-W″-X″-Y″-Z″;
wherein two -W′-X′-Y′-Z′ attached to the same atom optionally form a 3-14 membered cycloalkyl or 3-14 membered heterocycloalkyl group optionally substituted by 1, 2 or 3-W″-X″-Y″-Z″;
wherein -W-X-Y-Z is other than H;
wherein -W′-X′-Y′-Z′ is other than H;
wherein -W″-X″-Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl, wherein said C₁₋₆ alkyl, C₁₋₆ haloalkyl, C₂₋₆ alkenyl, C₂₋₆ alkynyl, aryl, cycloalkyl, heteroaryl or heterocycloalkyl; heterocycloalkyl, heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group; and
q is 1 or 2;
with the provisos:
(a) when L is absent and R² is methyl, then R³ is other than C₂₋₃ alkyl substituted by S(O)₂R^{b};
(b) when L is absent and R³ is methyl, then R² is other than ethyl substituted by NR^{c′}R^{d′};
(c) when L is S(O)₂ and Ar is 4-methylphenyl, then R³ is other than piperazin-1-yl which is 4-substituted by aryl;
(d) when L is S(O)₂ and q is 2, then Ar is other than aryl optionally substituted by 1, 2, 3, 4 or 5 -W-X-Y-Z;
(e) when L is C(O)NH and Ar is phenyl substituted by COOH, then R³ is other than heteroaryl substituted by 2 -W′-X′-Y′-Z′ or ethyl substituted by 2 -W′-X′-Y′-Z′; and
(f) R³ is other than piperidin-3-yl which is N-substituted by one —C(O)-(C₁₋₄ alkyl) or one —C(O)O(C₁₋₄ alkyl).

2. The compound of claim 1 wherein L is S(O)₂.

3. The compound of claim 1 wherien L is absent.

4. The compound of claim 1 wherein L is C(O).

5. The compound of claim 1 wherein L is C(O)NR^{L}.

6. The compound of claim 1 wherein L is C(O)O-(C₁₋₃ alkylene).

7. The compound of claim 1 having Formula IIa:

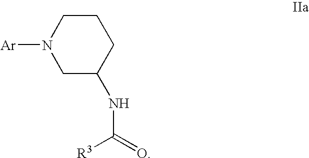

8. The compound of claim 7 wherein Ar is phenyl, pyridyl, pyrimidinyl, thiazolyl, each optionally substituted with 1 or 2 -W-X-Y-Z.

9. The compound of claim 7 wherein Ar is phenyl, pyridyl, pyrimidinyl, thiazolyl, each optionally substituted with 1 or 2 halo, nitro, cyano, amino, C₁₋₄ alkyl, C₁₋₄ alkoxy, C₁₋₄ haloalkyl, C₁₋₄ haloalkoxy, dialkylaminocarbonyl, dialkylaminocarbonylalkyloxy, cycloalkylcarbonylamino, cycloalkylcarbonyl(alkyl)amino, alkoxycarbonylamino, alkoxycarbonyl, alkylsulfonyl, alkylsulfonylamino, cycloalkylalkylcarbonylamino, aryl, heteroaryl, heterocycloalkyl, arylalkyloxy, cycloalkyloxy, heterocycloalkyloxy, acylamino, acyl(alkyl)amino, 1,2,3,6-tetrahydropyridinyl substituted by alkoxycarbonyl, 2-oxopiperidinyl, or 2-oxopyrrolidinyl;
wherein said aryl, heteroaryl, heterocycloalkyl, and heterocycloalkyloxy, are each optionally substituted by one or more halo, cyano, C₁₋₄ alkoxy, acyl, acylamino, alkylsulfonyl, cycloalkylaminocarbonyl, alkoxycarbonyl, or aminocarbonyl.

10. The compound of 7 whererin R³ is cyclopropyl, cyclobutyl, cyclopentyl, cyclohexyl, cycloheptyl, adamantyl, bicyclo[3.2.1]octanyl, norbornyl, 1,2,3,4-tetrahydronaphthyl, azepan-7-on-yl, 8-aza-bicyclo[3.2.1]octanyl, indolyl, quinolinyl, indol-3-ylmethyl, or phenyl, each optionally substituted by 1 or 2 -W′-X′-Y′-Z′.

11. The compound of 7 whererin R³ is cyclopropyl, cyclobutyl, cyclopentyl, cyclohexyl, cycloheptyl, adamantyl, bicyclo[3.2.1]octanyl, norbornyl, 1,2,3,4-tetrahydronaphthyl, azepan-7-on-yl, 8-aza-bicyclo[3.2.1]octanyl, or phenyl, each optionally substituted by 1 or 2 halo, OH, C₁₋₄ alkyl, C₁₋₄ alkoxy, hydroxylalkyl, aryl, aryloxy, heteroaryl, heteroarylalkyl, or alkylcarbonyloxy;
wherein said aryl, heteroaryl, heteroarylalkyl is optionally substituted by 1 or 2 C₁₋₄ alkyl or heterocycloalkyl optionally substituted by alkoxycarbonyl.

12. The compound of claim 1 having Formula IIIa:

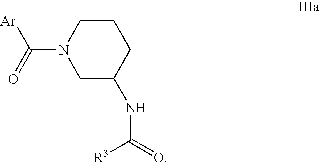

13. The compound of claim 1 having Formula IVa:

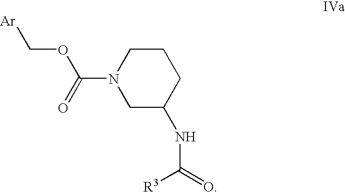

14. The compound of claim 1 having Formula Va:

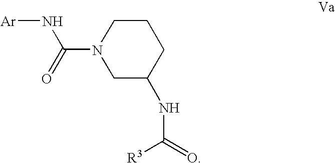

15. A compound of claim 1 having Formula I: 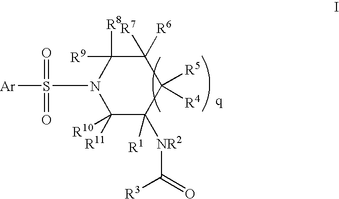
or pharmaceutically acceptable salt or prodrug thereof, wherein:
Ar is aryl or heteroaryl, each optionally substituted by 1, 2, 3, 4 or 5 -W-X-Y-Z;
R¹ is H, C(O)OR^{b′}, S(O)R^{a′}, S(O)NR^{c′}NR^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R² is H, C₁₋₆ alkyl, arylalkyl, heteroarylalkyl, cycloalkyl, cycloalkylalkyl, heterocycloalkyl or heterocycloalkylalkyl, each optionally substituted by 1, 2 or 3 R¹⁴;
R³ is H, C₁₋₆ alkyl, aryl, cycloalkyl or heteroaryl, each optionally substituted by 1, 2 or 3 -W′-X′-Y′-Z′;
R⁴, R⁵, R⁶, R⁷, R⁸, R⁹, R¹⁰ and R¹¹ are each, independently, H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
or R¹ and R² together with the carbon and nitrogen atoms to which they are attached form a 3-14 membered heterocycloalkyl group which is optionally substituted by 1, 2 or 3 R¹⁴;
or R¹ and R³ together with the carbon atoms to which they are attached and the intervening —NR²CO— moiety form a 4-14 membered heterocycloalkyl group which is optionally substituted by 1, 2 or 3 R¹⁴;
or R² and R³ together with the carbon and nitrogen atoms to which they are attached form a 3-14 membered heterocycloalkyl group which is optionally substituted by 1, 2 or 3 R¹⁴;
or R⁴ and R⁵ together with the carbon atom to which they are attached form a 3-14 membered cycloalkyl or 3-14 membered heterocycloalkyl group which is optionally substituted by 1, 2 or 3 R¹⁴;
or R⁶ and R⁷ together with the carbon atom to which they are attached form a 3-14 membered cycloalkyl or 3-14 membered heterocycloalkyl group which is optionally substituted by 1, 2 or 3 R¹⁴;
or R⁸ and R⁹ together with the carbon atom to which they are attached form a 3-14 membered cycloalkyl or 3-14 membered heterocycloalkyl group which is optionally substituted by 1, 2 or 3 R¹⁴;
or R¹⁰ and R¹¹ together with the carbon atom to which they are attached form a 3-14 membered cycloalkyl or 3-14 membered heterocycloalkyl group which is optionally substituted by 1, 2 or 3 R¹⁴;
or R⁴ and R⁶ together with the carbon atom to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atom to which they are attached form a 3-7 membered fused cycloalkyl group or 3-7 membered fused heterocycloalkyl group which is optionally substituted by 1, 2 or 3 R¹⁴;
R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two -W-X-Y-Z attached to the same atom optionally form a 3-14 membered cycloalkyl or heterocycloalkyl group optionally substituted by 1, 2 or 3-W″-X″-Y″-Z″;
wherein two -W′-X′-Y′-Z′ attached to the same atom optionally form a 3-14 membered cycloalkyl or heterocycloalkyl group optionally substituted by 1, 2 or 3-W″-X″-Y″-Z″;
wherein -W-X-Y-Z is other than H;
wherein -W′-X′-Y′-Z′ is other than H;
wherein -W″-X″-Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl, wherein said C₁₋₆ alkyl, C₁₋₆ haloalkyl, C₂₋₆ alkenyl, C₂₋₆ alkynyl, aryl, cycloalkyl, heteroaryl or heterocycloalkyl; heterocycloalkyl, heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group; and
q is 1 or 2.

16. The compound of claim 15 wherein Ar is aryl optionally substituted by 1, 2, 3, 4 or 5 -W-X-Y-Z.

17. The compound of claim 15 wherein Ar is phenyl or naphthyl, each optionally substituted by 1, 2, 3, 4 or 5 -W-X-Y-Z.

18. The compound of claim 15 wherein Ar is phenyl or naphthyl, each optionally substituted by 1, 2 or 3 halo; nitro; cyano; C₁₋₄ alkyl; C₁₋₄ haloalkyl; C₁₋₄ alkoxy; C₁₋₄ haloalkoxy; dialkylamino; dialkylaminocarbonyl; alkylsulfonyl; cycloalkyloxy; heteroaryloxy; aryloxy; cycloalkyl; heterocycloalkyl; phenyl optionally substituted by one or more halo, cyano, C₁₋₄ alkyl, C₁₋₄ alkoxy, or —NHC(O)-(C₁₋₄ alkyl); or pyridyl optionally substituted by one or more halo, cyano, C₁₋₄ alkyl, C₁₋₄ alkoxy, or —NHC(O)-(C₁₋₄ alkyl).

19. The compound of claim 15 wherein Ar is heteroaryl optionally substituted by 1, 2, 3, 4 or 5 -W-X-Y-Z.

20. The compound of claim 15 wherein Ar is pyridyl, pyrimidinyl, thienyl, thiazolyl, quinolinyl, 2,1,3-benzoxadiazolyl, isoquinolinyl or isoxazolyl, each optionally substituted by 1, 2, 3, 4 or 5 -W-X-Y-Z.

21. The compound of claim 15 wherein Ar is pyridyl, thienyl, or isoxazolyl, each optionally substituted by 1, 2, 3, 4 or 5 -W-X-Y-Z.

22. The compound of claim 15 wherein Ar is pyridyl, quinolinyl, 2,1,3-benzoxadiazolyl, isoquinolinyl, thienyl or isoxazolyl, each optionally substituted by 1, 2 or 3 halo, C₁₋₄ alkyl or aryloxy.

23. The compound of claim 15 wherein q is 1.

24. The compound of claim 15 wherein R³ is aryl, cycloalkyl, heteroaryl or heterocycloalkyl, each optionally substituted by 1, 2 or 3-W′-X′-Y′-Z′.

25. The compound of claim 15 wherein R³ is C₁₋₄ alkyl, aryl, cycloalkyl, heteroaryl or heterocycloalkyl, each optionally substituted by 1, 2 or 3 halo, C₁₋₄ alkyl, C₁₋₄ haloalkyl, C₁₋₄ alkoxy, phenyl, phenyl substituted by halo, phenyloxy, pyridyl, acyl, alkoxycarbonyl, alkylsulfonyl, arylsulfonyl, or arylsulfonyl optionally substituted by 1 or 2 halo or C₁₋₄ alkyl.

26. The compound of claim 15 wherein R³ is aryl or cycloalkyl, each optionally substituted by 1, 2 or 3 -W′-X′-Y′-Z′.

27. The compound of claim 15 wherein R³ is cycloheptyl, cyclohexyl, cyclopentyl, cyclopropyl, 1,2,3,4-tetrahydronaphthalenyl, norbornyl, or adamantyl, each optionally substituted by 1, 2 or 3 -W′-X′-Y′-Z′.

28. The compound of claim 15 wherein R³ is phenyl or naphthyl, each optionally substituted by 1, 2 or 3 -W′-X′-Y′-Z′.

29. The compound of claim 15 wherein R³ is phenyl or naphthyl, each optionally substituted by 1, 2 or 3 halo, C₁₋₄ alkyl, C₁₋₄ haloalkyl, C₁₋₄ alkoxy, C₁₋₄ haloalkoxy, C₂₋₈ alkoxyalkyl, aryl, aryloxy, pyridyl, or azepan-2-on-yl.

30. The compound of claim 15 wherein R³ is phenyl or naphthyl, each optionally substituted by 1, 2 or 3 halo, C₁₋₄ alkyl, C₁₋₄ alkoxy, C₁₋₄ haloalkyl, aryl or aryloxy.

31. The compound of claim 15 wherein R³ is heteroaryl or heterocycloalkyl, each optionally substituted by 1, 2 or 3 -W′-X′-Y′-Z′.

32. The compound of claim 15 wherein R³ is piperidinyl optionally substituted by 1, 2 or 3 -W′-X′-Y′-Z′.

33. The compound of claim 15 wherein R³ is piperidinyl optionally substituted by 1, 2 or 3 CO-(C₁₋₄ alkyl), C(O)O-(C₁₋₄ alkyl), SO₂-(C₁₋₄ alkyl), SO₂-aryl or SO₂-(aryl substituted by 1 or 2 halo or C₁₋₄ alkyl).

34. The compound of claim 15 wherein R³ is pyridyl optionally substituted by 1, 2 or 3 -W′-X′-Y′-Z′.

35. The compound of claim 15 wherein R³ is pyridyl.

36. The compound of claim 15 wherein R³ is 8-aza-bicyclo[3.2.1]octanyl, indolyl, morpholino, S-oxo-thiomorpholino, S,S-dioxo-thiomorpholino, or thiomorpholino, each optionally substituted by 1, 2 or 3 -W′-X′-Y′-Z′.

37. The compound of claim 15 wherein R⁴, R⁵, R⁶, R⁷, R⁸, R⁹, R¹⁰ and R¹¹ are each H.

38. The compound of claim 15 wherein R¹ is H.

39. The compound of claim 15 wherein R² is H.

40. A compound of claim 15 having Formula II:

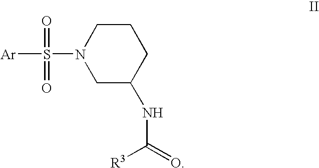

41. The compound of claim 40 wherein Ar is phenyl, naphthyl, pyridyl, thienyl, isoxazolyl, quinolinyl, isoquinolinyl, or 2,1,3-benzoxadiazolyl, each optionally substituted with 1 or 2 halo, cyano, nitro, C₁₋₄ alkyl, C₁₋₄ haloalkyl, C₁₋₄ alkoxy, C₁₋₄ haloalkoxy, aryloxy, heteroaryloxy, acylamino, alkylsulfonyl, or dialkylamino.

42. The compound of claim 40 wherein R³ is C₁₋₄ alkyl, cyclopropyl, cyclobutyl, cyclopentyl, cyclohexyl, cycloheptyl, adamantyl, phenyl, naphthyl, pyridyl, piperidinyl, morpholino, S-oxo-thiomorpholino, S,S-dioxo-thiomorpholino, thiomorpholino, or 8-aza-bicyclo[3.2.1]octanyl, each optionally substituted by 1 or 2 OH; C₁₋₄ alkyl; C₁₋₄ alkoxy; C₁₋₄ haloalkyl; phenyl; phenyloxy; arylsulfonyl optionally subsustituted by 1 or 2 halo or C₁₋₄ alkyl; chlorophenyl; alkylcarbonyl; alkoxycarbonyl; or alkylsulfonyl.

43. The compound of claim 15 wherein:
Ar is aryl or heteroaryl, each optionally substituted by 1, 2, 3, 4 or 5 -W-X-Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C-₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R² is H or C₁₋₆ alkyl;
R³ is H, C₁₋₆ alkyl, aryl, cycloalkyl, heteroaryl or heterocycloalkyl, each optionally substituted by 1, 2 or 3-W′-X′-Y′-Z′;
R⁴, R⁵, R⁶, R⁷, R⁸, R⁹, R¹⁰ and R¹¹ are each, independently, H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′, SR}^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴.

44. The compound of claim 15 wherein:
Ar is aryl or heteroaryl, each optionally substituted by 1, 2, 3, 4 or 5 -W-X-Y-Z;
R¹ is H;
R² is H;
R³ is C₁₋₆ alkyl, aryl, cycloalkyl, heteroaryl or heterocycloalkyl, each optionally substituted by 1, 2 or 3 -W′-X′-Y′-Z′; and
R⁴, R⁵, R⁶, R⁷, R⁸ , R⁹, R¹⁰ and R¹¹ are each H.

45. A compound of claim 15 wherein R³ is other than substituted piperidinyl.

46. A compound selected from:
N-(3R)-1-[(3-Chloro-2-methylphenyl)sulfonyl]piperidin-3-ylcyclohexanecarboxamide;
N-(3R)-1-[(2-Nitrophenyl)sulfonyl]piperidin-3-ylcyclohexanecarboxamide;
N-[(3R)-1-(2-Naphthylsulfonyl)piperidin-3-yl]cyclohexanecarboxamide;
N-(3R)-1-[(3-chlorophenyl)sulfonyl]piperidin-3-ylcyclohexanecarboxamide;
N-(3R)-1-[(4-propylphenyl)sulfonyl]piperidin-3-ylcyclohexanecarboxamide;
N-{(3R)-1-[(4-fluorophenyl)sulfonyl]piperidin-3-yl}cyclohexanecarboxamide;
N-{(3R)-1-[(3-methoxyphenyl)sulfonyl]piperidin-3-yl}cyclohexanecarboxamide;
N-(3R)-1-[(3-chloro-4-fluorophenyl)sulfonyl]piperidin-3-ylcyclohexanecarboxamide;
1-(4-Chlorophenyl)-N-[(3R)-1-(phenylsulfonyl)piperidin-3-yl]cyclohexanecarboxamide;
1-Methyl-N-[(3R)-1-(phenylsulfonyl)piperidin-3-yl]cyclohexanecarboxamide;
4-Hydroxy-N-[(3R)-1-(phenylsulfonyl)piperidin-3-yl]cyclohexanecarboxamide;
4-Methoxy-N-[(3R)-1-(phenylsulfonyl)piperidin-3-yl]cyclohexanecarboxamide;
N-[(3S)-1-(phenylsulfonyl)piperidin-3-yl]cyclohexanecarboxamide;
N-{(3S)-1-[(2-fluorophenyl)sulfonyl]piperidin-3-yl}cyclohexanecarboxamide;
N-{(3S)-1-[(2-Chlorophenyl)sulfonyl]piperidin-3-yl}cyclohexanecarboxamide;
N-{(3S)-1-[(2-Bromrophenyl)sulfonyl]piperidin-3-yl}cyclohexanecarboxamide;
N-{(3S)-1-[(2-Cyanophenyl)sulfonyl]piperidin-3-yl}cyclohexanecarboxamide;
N-{(3S)-1-[(2-Nitrophenyl)sulfonyl]piperidin-3-yl}cyclohexanecarboxamide;
N-{(3S)-1-[(2-methylphenyl)sulfonyl]piperidin-3-yl}cyclohexanecarboxamide;
N-((3S)-1-{[2-(trifluoromethyl)phenyl]sulfonyl}piperidin-3-yl)cyclohexanecarboxamide;
N-((3S)-1-{[2-(Trifluoromethoxy)phenyl]sulfonyl}piperidin-3-yl)cyclohexanecarboxamide;
N-{(3S)-1-[(2-Phenoxyphenyl)sulfonyl]piperidin-3-yl}cyclohexanecarboxamide;
N-{(3S)-1-[(3-Chlorophenyl)sulfonyl]piperidin-3-yl}cyclohexanecarboxamide;
N-{(3S)-1-[(3-Cyanophenyl)sulfonyl]piperidin-3-yl}cyclohexanecarboxamide;
N-{(3S)-1-[(3-Methylphenyl)sulfonyl]piperidin-3-yl}cyclohexanecarboxamide;
N-((3S)-1-{[3-(Trifluoromethyl)phenyl]sulfonyl}piperidin-3-yl)cyclohexanecarboxamide;
N-{(3S)-1-[(3-Phenoxyphenyl)sulfonyl]piperidin-3-yl}cyclohexanecarboxamide;
N-{(3S)-1-[(4-fluorophenyl)sulfonyl]piperidin-3-yl}cyclohexanecarboxamide;
N-{(3S)-1-[(4-chlorophenyl)sulfonyl]piperidin-3-yl}cyclohexanecarboxamide;
N-{(3S)-1-[(4-methoxyphenyl)sulfonyl]piperidin-3-yl}cyclohexanecarboxamide;
N-((3S)-1-{[4-(trifluoromethoxy)phenyl]sulfonyl}piperidin-3-yl)-cyclohexane-carboxamide;
N-(3S)-1-[(3-Chloro-2-methylphenyl)sulfonyl]piperidin-3-ylcyclohexanecarboxamide;
N-((3S)-1-{[4-(acetylamino)phenyl]sulfonyl}piperidin-3-yl)cyclohexanecarboxamide;
N-{(3S)-1-[(4-isopropylphenyl)sulfonyl]piperidin-3-yl}cyclohexanecarboxamide;
N-{(3S)-1-[(4-methylphenyl)sulfonyl]piperidin-3-yl}cyclohexanecarboxamide;
N-((3S)-1-{[4-(methylsulfonyl)phenyl]sulfonyl}piperidin-3-yl)cyclohexanecarboxamide;
N-((3S)-1-{[4-(pyridin-4-yloxy)phenyl]sulfonyl}piperidin-3-yl)cyclohexanecarboxamide;
N-((3S)-1-{[4-(pyridin-3-yloxy)phenyl]sulfonyl}piperidin-3-yl)cyclohexanecarboxamide;
N-{(3S)-1-[(4-tert-butylphenyl)sulfonyl]piperidin-3-yl}cyclohexanecarboxamide;
N-{(3S)-1-[(4-fluoro-2-methylphenyl)sulfonyl]piperidin-3-yl}cyclohexanecarboxamide;
N-{(3S)-1-[(2,3-dichlorophenyl)sulfonyl]piperidin-3-yl}cyclohexanecarboxamide;
N-{(3S)-1-[(2,6-dichlorophenyl)sulfonyl]piperidin-3-yl}cyclohexanecarboxamide;
N-{(3S)-1-[(2,5-dichlorophenyl)sulfonyl]piperidin-3-yl}cyclohexanecarboxamide;
N-{(3S)-1-[(3,4-dichlorophenyl)sulfonyl]piperidin-3-yl}cyclohexanecarboxamide;
N-{(3S)-1-[(3-chloro-4-fluorophenyl)sulfonyl]piperidin-3-yl}cyclohexanecarboxamide;
N-{(3S)-1-[(5-chloro-2-fluorophenyl)sulfonyl]piperidin-3-yl}cyclohexanecarboxamide;
N-{(3S)-1-[(3-chloro-2-fluorophenyl)sulfonyl]piperidin-3-yl}cyclohexanecarboxamide;
N-{(3S)-1-[(2,6-difluorophenyl)sulfonyl]piperidin-3-yl}cyclohexanecarboxamide;
N-{(3S)-1-[(3,4-dimethoxyphenyl)sulfonyl]piperidin-3-yl}cyclohexanecarboxamide;
N-{(3S)-1-[(2,5-dimethoxyphenyl)sulfonyl]piperidin-3-yl}cyclohexanecarboxamide;
N-[(3S)-1-(1-naphthylsulfonyl)piperidin-3-yl]cyclohexanecarboxamide;
N-[(3S)-1-(pyridin-3-ylsulfonyl)piperidin-3-yl]cyclohexanecarboxamide;
N-[(3S)-1-(2-thienylsulfonyl)piperidin-3-yl]cyclohexanecarboxamide;
N-{(3S)-1-[(3,5-dimethylisoxazol-4-yl)sulfonyl]piperidin-3-yl}cyclohexanecarboxamide;
N-{(3S)-1-[(4-phenoxypyridin-3-yl)sulfonyl]piperidin-3-yl}cyclohexanecarboxamide;
N-{(3S)-1-[(3-chloro-2-methylphenyl)sulfonyl]piperidin-3-yl}cyclopentanecarboxamide;
N-{(3S)-1-[(3-Chloro-2-methylphenyl)sulfonyl]piperidin-3-yl}adamantane-1-carboxamide;
N-{(3S)-1-[(3-Chloro-2-methylphenyl)sulfonyl]piperidin-3-yl}-2-methylpropanamide;
N-{(3S)-1-[(3-Chloro-2-methylphenyl)sulfonyl]piperidin-3-yl}-2,2-dimethylpropanamide;
N-{(3S)-1-[(3-chloro-2-methylphenyl)sulfonyl]piperidin-3-yl}-2,2-diphenylacetamide;
1-Acetyl-N-{(3S)-1-[(3-chloro-2-methylphenyl)sulfonyl]piperidin-3-yl}piperidine-4-carboxamide;
N-{(3S)-1-[(3-chloro-2-methylphenyl)sulfonyl]piperidin-3-yl}-1-(4-chlorophenyl)cyclopentanecarboxamide;
N-{(3S)-1-[(3-Chloro-2-methylphenyl)sulfonyl]piperidin-3-yl}-1-methylcyclohexanecarboxamide;
N-{(3S)-1-[(3-chloro-2-methylphenyl)sulfonyl]piperidin-3-yl}-3-methoxycyclohexanecarboxamide;
trans-N-{(3S)-1-[(3-Chloro-2-methylphenyl)sulfonyl]piperidin-3-yl}-3-methoxycyclohexanecarboxamide;
N-{(3S)-1-[(3-Chloro-2-methylphenyl)sulfonyl]piperidin-3-yl}-4-methoxycyclohexanecarboxamide;
trans-N-{(3S)-1-[(3-Chloro-2-methylphenyl)sulfonyl]piperidin-3-yl}-4-methoxycyclohexanecarboxamide;
N-{(3S)-1-[(3-chloro-2-methylphenyl)sulfonyl]piperidin-3-yl}-4-hydroxycyclohexanecarboxamide;
trans-N-{(3S)-1-[(3-Chloro-2-methylphenyl)sulfonyl]piperidin-3-yl}-4-hydroxycyclohexanecarboxamide;
N-{(3S)-1-[(3-Chloro-2-methylphenyl)sulfonyl]piperidin-3-yl}-1-phenylcyclopropanecarboxamide;
N-{(3S)-1-[(3-Chloro-2-methylphenyl)sulfonyl]piperidin-3-yl}biphenyl-2-carboxamide;
N-{(3S)-1-[(3-Chloro-2-methylphenyl)sulfonyl]piperidin-3-yl}cycloheptanecarboxamide;
tert-Butyl (3S)-3-[((3S)-1-[(3-chloro-2-methylphenyl)sulfonyl]piperidin-3-ylamino)carbonyl]piperidine-1-carboxylate;
(3S)-N-(3S)-1-[(3-Chloro-2-methylphenyl)sulfonyl]piperidin-3-yl-1-(methylsulfonyl)piperidine-3-carboxamide;
(3S)-N-(3S)-1-[(3-Chloro-2-methylphenyl)sulfonyl]piperidin-3-yl-1-(methylsulfonyl)piperidine-3-carboxamide;
(3S)-1-[(3-Chloro-2-methylphenyl)sulfonyl]-N-(3S)-1-[(3-chloro-2-methylphenyl)sulfonyl]piperidin-3-ylpiperidine-3-carboxamide;
N-{(3S)-1-[(3-Chloro-2-methylphenyl)sulfonyl]piperidin-3-yl}benzamide;
N-{(3S)-1-[(3-Chloro-2-methylphenyl)sulfonyl]piperidin-3-yl}-2-methylbenzamide;
N-{(3S)-1-[(3-Chloro-2-methylphenyl)sulfonyl]piperidin-3-yl}-2-chlorobenzamide;
N-{(3S)-1-[(3-Chloro-2-methylphenyl)sulfonyl]piperidin-3-yl}-3-fluorobenzamide;
N-{(3S)-1-[(3-Chloro-2-methylphenyl)sulfonyl]piperidin-3-yl}-3-methoxybenzamide;
N-{(3S)-1-[(3-Chloro-2-methylphenyl)sulfonyl]piperidin-3-yl}-3-(trifluoromethyl)benzamide;
N-{(3S)-1-[(3-Chloro-2-methylphenyl)sulfonyl]piperidin-3-yl}pyridine-2-carboxamide;
N-{(3S)-1-[(3-Chloro-2-methylphenyl)sulfonyl]piperidin-3-yl}pyridine-3-carboxamide;
N-{(3S)-1-[(3-Chloro-2-methylphenyl)sulfonyl]piperidin-3-yl}pyridine-4-carboxamide;
N-{(3S)-1-[(3-Chloro-2-methylphenyl)sulfonyl]piperidin-3-yl}-4-methoxybenzamide;
N-{(3S)-1-[(3-Chloro-2-methylphenyl)sulfonyl]piperidin-3-yl}-3-phenoxybenzamide;
N-{(3S)-1-[(3-Chloro-2-methylphenyl)sulfonyl]piperidin-3-yl}-1-naphthamide;
N-{(3S)-1-[(3-Chloro-2-methylphenyl)sulfonyl]piperidin-3-yl}-2-methoxybenzamide;
N-{(3S)-1-[(3-Chloro-2-methylphenyl)sulfonyl]piperidin-3-yl}-2,5-difluorobenzamide;
N-{(3S)-1-[(3-Chloro-2-methylphenyl)sulfonyl]piperidin-3-yl}-2-fluoro-4-(trifluoromethyl)benzamide;
N-{(3S)-1-[(3-Chloro-2-methylphenyl)sulfonyl]piperidin-3-yl}-4-fluoro-3-(trifluoromethyl)benzamide;
N-{(3S)-1-[(3-Chloro-2-methylphenyl)sulfonyl]piperidin-3-yl}-2-fluoro-5-(trifluoromethyl)benzamide;
N-{(3S)-1-[(3-Chloro-2-methylphenyl)sulfonyl]piperidin-3-yl}-3,5-difluorobenzamide;
N-{(3S)-1-[(3-Chloro-2-methylphenyl)sulfonyl]piperidin-3-yl}-2,6-difluorobenzamide;
4-Hydroxy-N-[(3S)-1-phenylpiperidin-3-yl]cyclohexanecarboxamide;
4-Methoxy-N-[(3S)-1-phenylpiperidin-3-yl]cyclohexanecarboxamide;
4-(Hydroxymethyl)-N-[(3S)-1-phenylpiperidin-3-yl]cyclohexanecarboxamide;
2-Hydroxy-N-[(3S)-1-phenylpiperidin-3-yl]bicyclo[3.2.1]octane-6-carboxamide;
N-[(3S)-1-Phenylpiperidin-3-yl]adamantane-1-carboxamide;
3-Hydroxy-N-[(3S)-1-phenylpiperidin-3-yl]adamantane-1-carboxamide;
N-[(3S)-1-Phenylpiperidin-3-yl]cyclohexanecarboxamide;
1-Methyl-N-[(3S)-1-phenylpiperidin-3-yl]cyclohexanecarboxamide;
4-Methyl-N-[(3S)-1-phenylpiperidin-3-yl]cyclohexanecarboxamide;
4-Ethyl-N-[(3S)-1-phenylpiperidin-3-yl]cyclohexanecarboxamide;
3-Methoxy-N-[(3S)-1-phenylpiperidin-3-yl]cyclohexanecarboxamide;
4-Methoxy-N-[(3S)-1-phenylpiperidin-3-yl]cyclohexanecarboxamide;
N-[(3S)-1-Phenylpiperidin-3-yl]bicyclo[2.2.1]heptane-2-carboxamide;
N-[(3S)-1-Phenylpiperidin-3-yl]cycloheptanecarboxamide;
N-[(3S)-1-Phenylpiperidin-3-yl]-1,2,3,4-tetrahydronaphthalene-2-carboxamide;
2-Methyl-N-[(3S)-1-phenylpiperidin-3-yl]benzamide;
5-Chloro-2-methyl-N-[(3S)-1-phenylpiperidin-3-yl]benzamide;
N-[(3S)-1-Phenylpiperidin-3-yl]biphenyl-4-carboxamide;
3-Methoxy-N-[(3S)-1-phenylpiperidin-3-yl]benzamide;
4-Methoxy-N-[(3S)-1-phenylpiperidin-3-yl]benzamide;
4-Phenoxy-N-[(3S)-1-phenylpiperidin-3-yl]benzamide;
2-(2-Methyl-1H-indol-3-yl)-N-[(3S)-1-phenylpiperidin-3-yl]acetamide;
N-[(3S)-1-Phenylpiperidin-3-yl]-1H-indole-3-carboxamide;
N-[(3S)-1-Phenylpiperidin-3-yl]-1H-indole-2-carboxamide;
1-Methyl-N-[(3S)-1-phenylpiperidin-3-yl]-1H-indole-2-carboxamide;
2-Methyl-N-[(3S)-1-phenylpiperidin-3-yl]quinoline-3-carboxamide;
N-{(3S)-1-[(3-Chloro-2-methylphenyl)sulfonyl]piperidin-3-yl}piperidine-1-carboxamide;
N-{(3S)-1-[(3-Chloro-2-methylphenyl)sulfonyl]piperidin-3-yl}-4-hydroxypiperidine-1-carboxamide;
N-{(3S)-1-[(3-Chloro-2-methylphenyl)sulfonyl]piperidin-3-yl}morpholine-4-carboxamide;
N-{(3S)-1-[(3-Chloro-2-methylphenyl)sulfonyl]piperidin-3-yl}thiomorpholine-4-carboxamide;
N-{(3S)-1-[(3-Chloro-2-fluorophenyl)sulfonyl]piperidin-3-yl}piperidine-1-carboxamide;
N-{(3S)-1-[(3-Chloro-2-fluorophenyl)sulfonyl]piperidin-3-yl}-4-hydroxypiperidine-1-carboxamide;
N-{(3S)-1-[(3-Chloro-2-fluorophenyl)sulfonyl]piperidin-3-yl}morpholine-4-carboxamide;
N-{(3S)-1-[(3-Chloro-2-fluorophenyl)sulfonyl]piperidin-3-yl}thiomorpholine-4-carboxamide;
N-{(3S)-1-[(2,6-Dichlorophenyl)sulfonyl]piperidin-3-yl}piperidine-1-carboxamide;
N-{(3S)-1-[(2,6-Dichlorophenyl)sulfonyl]piperidin-3-yl}-4-hydroxypiperidine-1-carboxamide;
N-{(3S)-1-[(2,6-Dichlorophenyl)sulfonyl]piperidin-3-yl}morpholine-4-carboxamide;
N-{(3S)-1-[(2,6-Dichlorophenyl)sulfonyl]piperidin-3-yl}thiomorpholine-4-carboxamide;
N-{(3S)-1-[(3-Chloro-2-fluorophenyl)sulfonyl]piperidin-3-yl}thiomorpholine-4-carboxamide 1-oxide;
N-{(3S)-1-[(3-Chloro-2-fluorophenyl)sulfonyl]piperidin-3-yl}thiomorpholine-4-carboxamide 1,1-dioxide;
N-{(3S)-1-[(3-Chloro-2-methylphenyl)sulfonyl]piperidin-3-yl}thiomorpholine-4-carboxamide 1,1-dioxide;
N-{(3S)-1-[(3-Chloro-2-methylphenyl)sulfonyl]piperidin-3-yl}thiomorpholine-4-carboxamide 1-oxide;
N-{(3S)-1-[(2,6-Dichlorophenyl)sulfonyl]piperidin-3-yl}thiomorpholine-4-carboxamide 1-oxide;
N-{(3S)-1-[(2,6-Dichlorophenyl)sulfonyl]piperidin-3-yl}thiomorpholine-4-carboxamide 1,1-dioxide;
4-Hydroxy-N-[(3S)-1-phenylpiperidin-3-yl]adamantane-1-carboxamide;
N-[(3S)-1-Phenylpiperidin-3-yl]-1-pyridin-4-ylcyclobutanecarboxamide;
N-[(3S)-1-Phenylpiperidin-3-yl]-1-pyridin-3-ylcyclobutanecarboxamide;
1-Phenyl-N-[(3S)-1-phenylpiperidin-3-yl]cyclopropanecarboxamide;
Methyl 4-{3-fluoro-4-[1-({[(3S)-1-phenylpiperidin-3-yl]amino}-carbonyl)cyclopropyl]phenyl}piperazine-1-carboxylate;
Benzyl (3S)-3-{[(4-hydroxy-1-adamantyl)carbonyl]amino}piperidine-1-carboxylate;
4-Hydroxy-N-{(3S)-1-[6-(trifluoromethyl)pyridin-2-yl]piperidin-3-yl}adamantane-1-carboxamide;
4-Hydroxy-N-{(3S)-1-[5-(trifluoromethyl)pyridin-2-yl]piperidin-3-yl}adamantane-1-carboxamide;
4-Hydroxy-N-[(3S)-1-(5-nitropyridin-2-yl)piperidin-3-yl]adamantane-1-carboxamide;
N-[(3S)-1-(5-Cyanopyridin-2-yl)piperidin-3-yl]-4-hydroxyadamantane-1-carboxamide;
6-((3S)-3-{[(4-Hydroxy-1-adamantyl)carbonyl]amino}piperidin-1-yl)-N,N-dimethylnicotinamide;
Methyl 6-((3S)-3-{[(4-hydroxy-1-adamantyl)carbonyl]amino}piperidin-1-yl)nicotinate;
4-Hydroxy-N-{(3S)-1-[4-(trifluoromethyl)phenyl]piperidin-3-yl}adamantane-1-carboxamide;
4-Hydroxy-N-{(3S)-1-[4-(trifluoromethoxy)phenyl]piperidin-3-yl}adamantane-1-carboxamide;
N-{(3S)-1-[4-(Benzyloxy)phenyl]piperidin-3-yl}-4-hydroxyadamantane-1-carboxamide;
N-[(3S)-1-(3-Fluoropyridin-4-yl)piperidin-3-yl]-4-hydroxyadamantane-1-carboxamide;
4-Hydroxy-N-[(3S)-1-(1,3-thiazol-2-yl)piperidin-3-yl]adamantane-1-carboxamide;
(3S)-3-{[(4-Hydroxy-1-adamantyl)carbonyl]amino}-N-phenylpiperidine-1-carboxamide;
N-[(3S)-1-Benzoylpiperidin-3-yl]-4-hydroxyadamantane-1-carboxamide;
4-Hydroxy-N-[(3S)-1-(4-pyridin-3-ylphenyl)piperidin-3-yl]adamantane-1-carboxamide;
N-{(3S)-1-[5 -(4-Chlorophenyl)pyridin-2-yl]piperidin-3-yl}-4-hydroxyadamantane-1-carboxamide;
4-Hydroxy-N-[(3S)-1-(4-pyridin-2-ylphenyl)piperidin-3-yl]adamantane-1-carboxamide;
(1S,5S)-3-Hydroxy-N-[(3S)-1-(1-naphthylsulfonyl)piperidin-3-yl]-8-azabicyclo[3.2.1]octane-8-carboxamide;
(1S,5S)-N-{(3S)-1-[(2,6-Dichlorophenyl)sulfonyl]piperidin-3-yl}-3-hydroxy-8-azabicyclo[3.2.1]octane-8-carboxamide;
(1S,5S)-N-{(3S)-1-[(3-Chloro-2-fluorophenyl)sulfonyl]piperidin-3-yl}-3-hydroxy-8-azabicyclo[3.2.1]octane-8-carboxamide;
(1S,5S)-N-{(3S)-1-[(3-chloro-2-methylphenyl)sulfonyl]piperidin-3-yl}-3-hydroxy-8-azabicyclo[3.2.1]octane-8-carboxamide;
(1S,5S)-N-{(3S)-1-[(3-chlorophenyl)sulfonyl]piperidin-3-yl}-3-hydroxy-8-azabicyclo[3.2.1]octane-8-carboxamide;
(1S,5S)-3-Hydroxy-N-{(3S)-1-[(3-methylphenyl)sulfonyl]piperidin-3-yl}-8-azabicyclo[3.2.1]octane-8-carboxamide;
(1S,5S)-N-{(3S)-1-[(2-Fluorophenyl)sulfonyl]piperidin-3-yl}-3-hydroxy-8-azabicyclo[3.2.1]octane-8-carboxamide;
(1S,5S)-3-Hydroxy-N-{(3S)-1-[(2-methylphenyl)sulfonyl]piperidin-3-yl}-8-azabicyclo[3.2.1]octane-8-carboxamide;
N-((3S)-1-{4-[2-(Diethylamino)-2-oxoethoxy]phenyl}piperidin-3-yl)-4-hydroxyadamantane-1-carboxamide;
N-((3S)-1-{4-[(Cyclopropylcarbonyl)(methyl)amino]phenyl}piperidin-3-yl)-4-hydroxyadamantane-1-carboxamide;
7-Oxo-N-{(3S)-1-[4-(trifluoromethoxy)phenyl]piperidin-3-yl}azepane-4-carboxamide;
7-Oxo-N-{(3S)-1-[5-(trifluoromethyl)pyridin-2-yl]piperidin-3-yl}azepane-4-carboxamide;
7-Oxo-N-[(3S)-1-phenylpiperidin-3-yl]azepane-4-carboxamide;
N-[(3S)-1-(2-Fluoro-4-pyridin-4-ylphenyl)piperidin-3-yl]-4-hydroxyadamantane-1-carboxamide;
4-Hydroxy-N-[(3S)-1-(1-naphthylsulfonyl)piperidin-3-yl]piperidine-1-carboxamide;
N-{(3S)-1-[4-(Difluoromethoxy)phenyl]piperidin-3-yl}-4-hydroxyadamantane-1-carboxamide;
N-{(3S)-1-[3-Fluoro-5 -(trifluoromethyl)phenyl]piperidin-3-yl}-4-hydroxyadamantane-1-carboxamide;
N-{(3S)-1-[3-(Difluoromethoxy)phenyl]piperidin-3-yl}-4-hydroxyadamantane-1-carboxamide;
4-Hydroxy-N-{(3S)-1-[5-(trifluoromethyl)pyridin-2-yl]piperidin-3-yl}adamantane-1-carboxamide;
N-{(3S)-1-[3-Chloro-5-(trifluoromethyl)pyridin-2-yl]piperidin-3-yl}-4-hydroxyadamantane-1-carboxamide;
4-Hydroxy-N-{(3S)-1-[6-methyl-4-(trifluoromethyl)pyridin-2-yl]piperidin-3-yl}adamantane-1-carboxamide;
4-Hydroxy-N-[(3S)-1-(6-methylpyridin-2-yl)piperidin-3-yl]adamantane-1-carboxamide;
N-[(3S)-1-(6-Fluoropyridin-2-yl)piperidin-3-yl]-4-hydroxyadamantane-1-carboxamide;
4-Hydroxy-N-[(3S)-1-(4-methylpyridin-2-yl)piperidin-3-yl]adamantane-1-carboxamide;
4-Hydroxy-N-[(3S)-1-(4-methoxypyridin-2-yl)piperidin-3-yl]adamantane-1-carboxamide;
4-Hydroxy-N-[(3S)-1-(6-methoxypyridin-2-yl)piperidin-3-yl]adamantane-1-carboxamide;
N-[(3S)-1-(5-Fluoropyridin-2-yl)piperidin-3-yl]-4-hydroxyadamantane-1-carboxamide;
4-Hydroxy-N-[(3S)-1-(5-methylpyridin-2-yl)piperidin-3-yl]adamantane-1-carboxamide;
N-[(3S)-1-(5-Chloropyridin-2-yl)piperidin-3-yl]-4-hydroxyadamantane-1-carboxamide;
N-[(3S)-1-(2,5-Difluoropyridin-3-yl)piperidin-3-yl]-4-hydroxyadamantane-1-carboxamide;
N-[(3S)-1-(3,5-Difluoropyridin-2-yl)piperidin-3-yl]-4-hydroxyadamantane-1-carboxamide;
N-{(3S)-1-[4-(Cyclohexyloxy)phenyl]piperidin-3-yl}-4-hydroxyadamantane-1-carboxamide;
N-{(3S)-1-[4-(Cyclopentyloxy)phenyl]piperidin-3-yl}-4-hydroxyadamantane-1-carboxamide;
4-Hydroxy-N-[(3S)-1-phenylpiperidin-3-yl]piperidine-1-carboxamide;
(1S,5 S)-3-Hydroxy-N-[(3S)-1-phenylpiperidin-3-yl]-8-azabicyclo[3.2.1]octane-8-carboxamide;
N-[(3S)-1-(3,4′-bipyridin-6-yl)piperidin-3-yl]-4-hydroxyadamantane-1-carboxamide;
N-((3S)-1-{5-[4-(Acetylamino)phenyl]pyridin-2-yl}piperidin-3-yl)-4-hydroxyadamantane-1-carboxamide;
N-{(3S)-1-[5-(4-cyanophenyl)pyridin-2-yl]piperidin-3-yl}-4-hydroxyadamantane-1-carboxamide;
4-Hydroxy-N-{(3S)-1-[4-(2-oxopyrrolidin-1-yl)phenyl]piperidin-3-yl}adamantane-1-carboxamide;
4-Hydroxy-N-{(3S)-1-[5 -(4-methoxyphenyl)pyridin-2-yl]piperidin-3-yl}adamantane-1-carboxamide;
Ethyl [4-((3S)-3-{[(4-Hydroxy-1-adamantyl)carbonyl]amino}piperidin-1-yl)phenyl]methylcarbamate;
N-[(3S)-1-(5-{4-[(Cyclopropylamino)carbonyl]phenyl}pyridin-2-yl)piperidin-3-yl]-4-hydroxyadamantane-1-carboxamide;
N-[(3S)-1-(6′-Fluoro-3,3′-bipyridin-6-yl)piperidin-3-yl]-4-hydroxyadamantane-1-carboxamide;
tert-Butyl 4-[4-((3S)-3-{[(4-hydroxy-1-adamantyl)carbonyl]amino}piperidin-1-yl)phenoxy]piperidine-1-carboxylate;
4-Hydroxy-N-[(3S)-1-(6′-methoxy-3,3′-bipyridin-6-yl)piperidin-3-yl]adamantane-1-carboxamide;
6′-((3S)-3-{[(4-Hydroxy-1-adamantyl)carbonyl]amino}piperidin-1-yl)-3,3 ′-bipyridine-6-carboxamide;
4-Hydroxy-N-[(3S)-1-(quinolin-8-ylsulfonyl)piperidin-3-yl]piperidine-1-carboxamide;
N-((3S)-1-{[5-(Dimethylamino)-1-naphthyl]sulfonyl}piperidin-3-yl)-4-hydroxypiperidine-1-carboxamide;
(3-exo)-N-((3S)-1-{[5-(Dimethylamino)-1-naphthyl]sulfonyl}piperidin-3-yl)-3-hydroxy-8-azabicyclo[3.2.1]octane-8-carboxamide;
(3-endo)-N-((3S)-1-{[5-(Dimethylamino)-1-naphthyl]sulfonyl}piperidin-3-yl)-3-hydroxy-8-azabicyclo[3.2.1]octane-8-carboxamide;
3-Hydroxy-N-[(3S)-1-(quinolin-8-ylsulfonyl)piperidin-3-yl]-8-azabicyclo[3.2.1]octane-8-carboxamide;
N-[(3S)-1-(2-Fluorophenyl)piperidin-3-yl]-3-hydroxy-8-azabicyclo[3.2.1]octane-8-carboxamide;
N-[(3S)-1-(4-Fluorophenyl)piperidin-3-yl]-3-hydroxy-8-azabicyclo[3.2.1]octane-8-carboxamide;
(3-endo)-N-[(3S)-1-(4-Cyanophenyl)piperidin-3-yl]-3-hydroxy-8-azabicyclo[3.2.1]octane-8-carboxamide;
(3-endo)-3-Hydroxy-N-{(3S)-1-[4-(methylsulfonyl)phenyl]piperidin-3-yl}-8-azabicyclo[3.2.1]octane-8-carboxamide;
(3-endo)-3-Hydroxy-N-{(3S)-1-[4-(trifluoromethoxy)phenyl]piperidin-3-yl}-8-azabicyclo[3.2.1]octane-8-carboxamide;
N-{(3S)-1-[(4-Chloro-1-naphthyl)sulfonyl]piperidin-3-yl}-4-hydroxypiperidine-1-carboxamide;
N-[(3S)-1-(5-Ethylpyrimidin-2-yl)piperidin-3-yl]-4-hydroxyadamantane-1-carboxamide;
4-Hydroxy-N-{(3S)-1-[4-(trifluoromethyl)pyrimidin-2-yl]piperidin-3-yl}adamantane-1-carboxamide;
N-[(3S)-1-(2-Chloropyrimidin-4-yl)piperidin-3-yl]-4-hydroxyadamantane-1-carboxamide;
N-[(3S)-1-(4-Chloropyrimidin-2-yl)piperidin-3-yl]-4-hydroxyadamantane-1-carboxamide;
4-Hydroxy-N-[(3S)-1-(4-pyridin-4-ylphenyl)piperidin-3-yl]adamantane-1-carboxamide;
N-{(3S)-1-[4-(3-Fluoropyridin-4-yl)phenyl]piperidin-3-yl}-4-hydroxyadamantane-1-carboxamide;
4-Hydroxy-N-[(3S)-1-(isoquinolin-5-ylsulfonyl)piperidin-3-yl]piperidine-1-carboxamide;
(3-endo)-3-Hydroxy-N-[(3S)-1-(isoquinolin-5-ylsulfonyl)piperidin-3-yl]-8-azabicyclo[3.2.1]octane-8-carboxamide;
(3-endo)-3-Hydroxy-N-[(3S)-1-(2-naphthylsulfonyl)piperidin-3-yl]-8-azabicyclo[3.2.1]octane-8-carboxamide;
(3-exo)-3-hydroxy-N-[(3S)-1-(2-naphthylsulfonyl)piperidin-3-yl]-8-azabicyclo[3.2.1]octane-8-carboxamide;
(3-exo)-N-{(3S)-1-[(4-Chloro-1-naphthyl)sulfonyl]piperidin-3-yl}-3-hydroxy-8-azabicyclo[3.2.1]octane-8-carboxamide;
(3-endo)-N-{(3S)-1-[(4-Chloro-1-naphthyl)sulfonyl]piperidin-3-yl}-3-hydroxy-8-azabicyclo[3.2.1]octane-8-carboxamide;
4-Hydroxy-N-[(3S)-1-(2-naphthylsulfonyl)piperidin-3-yl]piperidine-1-carboxamide;
N-[(3S)-1-(2,1,3-Benzoxadiazol-4-ylsulfonyl)piperidin-3-yl]-4-hydroxypiperidine-1-carboxamide;
(3-endo)-N-[(3S)-1-(2,1,3-Benzoxadiazol-4-ylsulfonyl)piperidin-3-yl]-3-hydroxy-8-azabicyclo[3.2.1]octane-8-carboxamide;
6-((3S)-3-{[(4-Hydroxy-1-adamantyl)carbonyl]amino}piperidin-1-yl)-N,N-dimethylnicotinamide;
tert-Butyl 6-[(3S)-3-({[4-(acetyloxy)-1-adamantyl]carbonyl}amino)piperidin-1-yl]-3′,6′-dihydro-3,4′-bipyridine-1′(2′H)-carboxylate;
Benzyl (3S)-3-{[(5-oxo-4-azatricyclo[4.3.1.1(3,8)]undec-1-yl)carbonyl]amino}piperidine-1-carboxylate;
(3-endo)-3-Hydroxy-N-[(3S)-1-(4-nitrophenyl)piperidin-3-yl]-8-azabicyclo[3.2.1]octane-8-carboxamide;
N-((3S)-1-{4-[(1-Acetylpiperidin-4-yl)oxy]phenyl}piperidin-3-yl)-4-hydroxyadamantane-1-carboxamide;
Methyl 4-[4-((3S)-3-{[(4-hydroxy-1-adamantyl)carbonyl]amino}piperidin-1-yl)phenoxy]piperidine-1-carboxylate;
4-Hydroxy-N-[(3S)-1-(4-{[1-(methylsulfonyl)piperidin-4-yl]oxy}phenyl)piperidin-3-yl]adamantane-1-carboxamide;
N-((3S)-1-{4-[Acetyl(methyl)amino]phenyl}piperidin-3-yl)-4-hydroxyadamantane-1-carboxamide;
(3-endo)-N-[(3S)-1-(4-Aminophenyl)piperidin-3-yl]-3-hydroxy-8-azabicyclo[3.2.1]octane-8-carboxamide;
(3-endo)-3-Hydroxy-N-((3S)-1-{4-[(methylsulfonyl)amino]phenyl}piperidin-3-yl)-8-azabicyclo[3.2.1]octane-8-carboxamide;
Ethyl {4-[(3S)-3-({[(3-endo)-3-hydroxy-8-azabicyclo[3.2.1]oct-8-yl]carbonyl}amino)piperidin-1-yl]phenyl}carbamate;
(3-endo)-3-Hydroxy-N-{(3S)-1-[4-(2-oxopiperidin-1-yl)phenyl]piperidin-3-yl}-8-azabicyclo[3.2.1]octane-8-carboxamide;
N-{(3S)-1-[4-(Acetylamino)phenyl]piperidin-3-yl}-4-hydroxyadamantane-1-carboxamide;
N-{(3S)-1-[4-(Acetylamino)phenyl]piperidin-3-yl}-4-oxoadamantane-1-carboxamide;
N-((3S)-1-{4-[(Cyclopropylcarbonyl)amino]phenyl}piperidin-3-yl)-4-hydroxyadamantane-1-carboxamide;
4-Hydroxy-4-methyl-N-{(3S)-1-[5-(trifluoromethyl)pyridin-2-yl]piperidin-3-yl}adamantane-1-carboxamide;
Methyl [4-((3S)-3-{[(4-hydroxy-1-adamantyl)carbonyl]amino}piperidin-1-yl)phenyl]carbamate;
(3-endo)-3-Hydroxy-N-{(3S)-1-[4-(trifluoromethyl)phenyl]piperidin-3-yl}-8-azabicyclo[3.2.1]octane-8-carboxamide;
(3-endo)-N-[(3S)-1-Biphenyl-4-ylpiperidin-3-yl]-3-hydroxy-8-azabicyclo[3.2.1]octane-8-carboxamide;
(3-endo)-N-((3S)-1-{4-[(Cyclopropylacetyl)amino]phenyl}piperidin-3-yl)-3-hydroxy-8-azabicyclo[3.2.1]octane-8-carboxamide;
(3-endo)-3-Hydroxy-N-{(3S)-1-[4-(2-oxopyrrolidin-1-yl)phenyl]piperidin-3-yl}-8-azabicyclo[3.2.1]octane-8-carboxamide;
(3-endo)-3-Hydroxy-N-{(3S)-1-[5-(trifluoromethyl)pyridin-2-yl]piperidin-3-yl}-8-azabicyclo[3.2.1]octane-8-carboxamide;
(3-endo)-N-[(3S)-1-(6-Fluoropyridin-2-yl)piperidin-3-yl]-3-hydroxy-8-azabicyclo[3.2.1]octane-8-carboxamide;
or pharmaceutically acceptable salt thereof.

47. A composition comprising a compound of claim 1 and a pharmaceutically acceptable carrier.

48. A method of modulating 11βHSD1 or MR comprising contacting said 11βHSD1 or MR with a compound of a compound of claim 1.

49. The method of claim 48 wherein said modulating is inhibiting.

50. A method of treating a disease in a patient, wherein said disease is associated with expression or activity of 11βHSD1 or MR, comprising administering to said patient a therapeutically effective amount of a compound of claim 1.

51. The method of claim 50 wherein said disease is obesity, diabetes, glucose intolerance, insulin resistance, hyperglycemia, hypertension, hyperlipidemia, cognitive impairment, depression, dementia, glaucoma, cardiovascular disorders, osteoporosis, inflammation, a cardiovascular, renal or inflammatory disease, heart failure, atherosclerosis, arteriosclerosis, coronary artery disease, thrombosis, angina, peripheral vascular disease, vascular wall damage, stroke, dyslipidemia, hyperlipoproteinaemia, diabetic dyslipidemia, mixed dyslipidemia, hypercholesterolemia, hypertriglyceridemia, metabolic syndrome or general aldosterone-related target organ damage.
